SEQUENCE LISTING (302 sequences)

SEQ ID NO: 1 - protein, 22 aa, synthetic construct
LGVFFSLLFLAALILTLFLGFL

SEQ ID NO: 2 - protein, 21 aa, synthetic construct
IAAIVIAALLATCVVLALVVV

SEQ ID NO: 3 - protein, 22 aa, synthetic construct
GAGLAGFFLGILAASLTLILIG

SEQ ID NO: 4 - protein, 23 aa, synthetic construct
VWVIAVIVIAIVVGVAVICVVPY

SEQ ID NO: 5 - protein, 22 aa, synthetic construct
VTTVGVAAGLLAAASLVGILLA

SEQ ID NO: 6 - protein, 25 aa, synthetic construct
GLLLLLLLVTAALVALVALGVLYAD

SEQ ID NO: 7 - protein, 21 aa, synthetic construct
WYFLIVLPVLLVIVIFAAVGV

SEQ ID NO: 8 - protein, 21 aa, synthetic construct
ATVVIVVCVSFLVFMIILGVF

SEQ ID NO: 9 - protein, 21 aa, synthetic construct
IATVVIIISVCMLVFVVAMGV

SEQ ID NO: 10 - protein, 21 aa, synthetic construct
VVGGAGATALVFLSFCVIFVV

SEQ ID NO: 11 - protein, 21 aa, synthetic construct
WQVIWMMAAVMLVLTVVLGLY

SEQ ID NO: 12 - protein, 23 aa, synthetic construct
LLGAMAATLVVICTAVIVVVALT

SEQ ID NO: 13 - protein, 21 aa, synthetic construct
LPWMIVLFLLLVLVVIVVCSI

SEQ ID NO: 14 - protein, 23 aa, synthetic construct
VLLGVGLAVVVSLTLTAVILVLT

SEQ ID NO: 15 - protein, 21 aa, synthetic construct
WKIVAFTATSVLLVLLLVILA

SEQ ID NO: 16 - protein, 21 aa, synthetic construct
AIPLVLLVVLVFAGMGIFAAF

SEQ ID NO: 17 - protein, 21 aa, synthetic construct
KAVSGSIVLVIVLAGVGVLVW

SEQ ID NO: 18 - protein, 21 aa, synthetic construct
VVVLGMCTMFIVVCALVGVLV

SEQ ID NO: 19 - protein, 21 aa, synthetic construct
VELAGGLGAILLLLVCLVTIY

SEQ ID NO: 20 - protein, 21 aa, synthetic construct
FGVISFIVILVVVVIILVGVV

SEQ ID NO: 21 - protein, 21 aa, synthetic construct
FMYVAAAAFVLLFFVGCGVLL

SEQ ID NO: 22 - protein, 5 aa, synthetic construct
KRKSR

SEQ ID NO: 23 - protein, 4 aa, synthetic construct
KRKR

SEQ ID NO: 24 - protein, 4 aa, synthetic construct
RRKH

SEQ ID NO: 25 - protein, 16 aa, synthetic construct
RRRRREQRLKRLRDAK

SEQ ID NO: 26 - protein, 9 aa, synthetic construct
KRKKQRSRR

SEQ ID NO: 27 - protein, 16 aa, synthetic construct
RSRRAQRQRQQRQRDR

SEQ ID NO: 28 - protein, 8 aa, synthetic construct
KQRRAKEK

SEQ ID NO: 29 - protein, 9 aa, synthetic construct
KRRQQKIRK

SEQ ID NO: 30 - protein, 9 aa, synthetic construct
RNARRPSRK

SEQ ID NO: 31 - protein, 8 aa, synthetic construct
RRRKGSRK

SEQ ID NO: 32 - protein, 9 aa, synthetic construct
SKKRRQRQR

SEQ ID NO: 33 - protein, 7 aa, synthetic construct
SKRKRTH

SEQ ID NO: 34 - protein, 10 aa, synthetic construct
SRKRNKTGKH

SEQ ID NO: 35 - protein, 9 aa, synthetic construct
SRRRGKTNH

SEQ ID NO: 36 - protein, 11 aa, synthetic construct
SKRRRSQRNRR

SEQ ID NO: 37 - protein, 6 aa, synthetic construct
SRKRRR

SEQ ID NO: 38 - protein, 6 aa, synthetic construct
KRKRKH

SEQ ID NO: 39 - protein, 43 aa, synthetic construct
RKRRRQHGQLWFPEGFKVSEASKKKRREPLGEDSVGLKPLKNA

SEQ ID NO: 40 - protein, 27 aa, synthetic construct
AIPLVLLVVLVFAGMGIFAAFKRKRKH

SEQ ID NO: 41 - protein, 29 aa, synthetic construct
WQVIWMMAAVMLVLTVVLGLYKKRRQRQR

SEQ ID NO: 42 - protein, 27 aa, synthetic construct
KAVSGSIVLVIVLAGVGVLVWSRKRRR

SEQ ID NO: 43 - protein, 29 aa, synthetic construct
VWVIAVIVIAIVVGVAVICVVPYSRKRRR

SEQ ID NO: 44 - protein, 27 aa, synthetic construct
VVVLGMCTMFIVVCALVGVLVKRKRKH

SEQ ID NO: 45 - protein, 27 aa, synthetic construct
VELAGGLGAILLLLVCLVTIYSRKRRR

SEQ ID NO: 46 - protein, 27 aa, synthetic construct
FGVISFIVILVVVVIILVGVVSRKRRR

SEQ ID NO: 47 - protein, 28 aa, synthetic construct
FMYVAAAAFVLLFFVGCGVLLSKRKRTH

SEQ ID NO: 48 - protein, 31 aa, synthetic construct
FMYVAAAAFVLLFFVGCGVLLSRKRNKTGKH

SEQ ID NO: 49 - protein, 28 aa, synthetic construct
GAGLAGFFLGILAASLTLILIGKRKRKH

SEQ ID NO: 50 - protein, 30 aa, synthetic construct
WQVIWMMAAVMLVLTVVLGLYKRKKQRSRR

SEQ ID NO: 51 - protein, 30 aa, synthetic construct
VVGGAGATALVFLSFCVIFVVKRKKQRSRR

SEQ ID NO: 52 - protein, 27 aa, synthetic construct
WQVIWMMAAVMLVLTVVLGLYSRKRRR

SEQ ID NO: 53 - protein, 30 aa, synthetic construct
FMYVAAAAFVLLFFVGCGVLLSRRRGKTNH

SEQ ID NO: 54 - protein, 32 aa, synthetic construct
FMYVAAAAFVLLFFVGCGVLLSKRRRSQRNRR

SEQ ID NO: 55 - protein, 30 aa, synthetic construct
FMYVAAAAFVLLFFVGCGVLLSKKRRQRQR

SEQ ID NO: 56 - protein, 37 aa, synthetic construct
WYFLIVLPVLLVIVIFAAVGVRSRRAQRQRQQRQRDR

SEQ ID NO: 57 - protein, 65 aa, synthetic construct
FMYVAAAAFVLLFFVGCGVLLSRKRRRQHGQLWFPEGFKVSEASKKKRREPLGEDSVGLKPLKNA

SEQ ID NO: 58 - DNA, 81 bp, synthetic construct
  source 1..81: mol_type = other DNA
gctatacccttagtactactcgttgttcttgtctttgccggaatggggatattcgcagccttcaaacgcaaacgaaaacac

SEQ ID NO: 59 - DNA, 87 bp, synthetic construct
  source 1..87: mol_type = other DNA
tggcaagtgatctggatgatggccgctgtgatgctcgtgctgacagtcgtcctgggcctctacaaaaagaggagacagcgccagcgg

SEQ ID NO: 60 - DNA, 81 bp, synthetic construct
  source 1..81: mol_type = other DNA
aaagctgtttccgggtcaatagttcttgttatagttttagccggagtcggcgtactcgtctggagcagaaaacgccggcga

SEQ ID NO: 61 - DNA, 87 bp, synthetic construct
  source 1..87: mol_type = other DNA
gtttgggttatagccgttatagttatagcaatagtcgtcggcgtcgctgttatatgtgtagtaccctacagccgaaaaagacggcgc

SEQ ID NO: 62 - DNA, 81 bp, synthetic construct
  source 1..81: mol_type = other DNA
gttgttgttctcgggatgtgcacaatgtttatagttgtttgtgccttagtcggcgtacttgtcaaacgaaaacgcaaacac

SEQ ID NO: 63 - DNA, 81 bp, synthetic construct
  source 1..81: mol_type = other DNA
gtagagttagccggagggctaggcgcaatacttctcctccttgtttgccttgtcactatatacagcagaaaacgacgccgg

SEQ ID NO: 64 - DNA, 81 bp, synthetic construct
  source 1..81: mol_type = other DNA
ttcggcgtcataagttttatagtaatactcgttgttgtcgtcataatacttgtaggagttgttagccggaaacgcagacga

SEQ ID NO: 65 - DNA, 84 bp, synthetic construct
  source 1..84: mol_type = other DNA
tttatgtacgttgctgccgcagcctttgtacttctcttcttcgttgggtgcggcgtcttactaagcaaacgcaaaagaacccac

SEQ ID NO: 66 - DNA, 93 bp, synthetic construct
  source 1..93: mol_type = other DNA
ttcatgtacgttgctgccgcagcctttgtattactctttttcgttgggtgcggagtccttctcagccggaaacgcaacaaaaccggcaaacac

SEQ ID NO: 67 - DNA, 84 bp, synthetic construct
  source 1..84: mol_type = other DNA
ggtgcaggcttagctgggtttttcctcggaatactcgccgccagccttaccctaatacttataggcaaacgcaaacgaaaacac

SEQ ID NO: 68 - DNA, 90 bp, synthetic construct
  source 1..90: mol_type = other DNA
tggcaagttatctggatgatggccgctgtgatgctagtgctgacagtcgtgctgggcttatacaagcggaaaaagcagcgcagcagaagg

SEQ ID NO: 69 - DNA, 90 bp, synthetic construct
  source 1..90: mol_type = other DNA
gtggtcggtggcgcgggagctaccgccctcgtgttcctgtccttttgcgtgatcttcgttgtaaaaaggaagaagcagcggagcagacgc

SEQ ID NO: 70 - DNA, 81 bp, synthetic construct
  source 1..81: mol_type = other DNA
tggcaggttatatggatgatggctgccgtaatgttagttctaaccgtcgtcctcggcctttacagccggaaacgacgcaga

SEQ ID NO: 71 - DNA, 90 bp, synthetic construct
  source 1..90: mol_type = other DNA
ttcatgtacgttgctgccgcagcctttgtattactctttttcgttgggtgcggagtccttctaagccgaagacgcggcaaaacaaaccac

SEQ ID NO: 72 - DNA, 96 bp, synthetic construct
  source 1..96: mol_type = other DNA
ttcatgtacgtagctgccgcagccttcgttcttctcttttttgttgggtgcggcgtcttactaagcaaaagacgcagatcccagcggaaccgtcga

SEQ ID NO: 73 - DNA, 90 bp, synthetic construct
  source 1..90: mol_type = other DNA
tttatgtacgttgctgccgcagccttcgtacttctcttctttgttgggtgcggcgtcctattaagcaaaaaacgacgccagagacaacgg

SEQ ID NO: 74 - DNA, 111 bp, synthetic construct
  source 1..111: mol_type = other DNA
tggtattttcttatcgtgctcccggtactgctagtgatcgtgattttcgctgccgttggcgtgcgaagcagaagggcacagcgccagagacaacagaggcagcgggaccgt

SEQ ID NO: 75 - DNA, 195 bp, synthetic construct
  source 1..195: mol_type = other DNA
ttcatgtacgtggcggcggccgcctttgtgcttctgttcttcgtgggctgcggggtgctgctgtcccgtaaacgcagacgtcaacacggtcaactgtggtttccagaaggttttaaggtctccgaagcaagtaagaagaaaagacgtgaaccactgggagaagatagcgtcggtctgaaaccactcaagaatgcc

SEQ ID NO: 76 - protein, 243 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 77 - protein, 813 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 78 - protein, 240 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 79 - protein, 466 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 283 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAM

SEQ ID NO: 81 - protein, 191 aa, synthetic construct
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 82 - protein, 477 aa, synthetic construct
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 83 - protein, 27 aa, synthetic construct
TTTPAPRPPTPAPTIASQPLSLRPEAC

SEQ ID NO: 84 - protein, 45 aa, synthetic construct
TTTPAPRPPTPAPTIASQPLSLRPEACRPAAGGAVHTRGLDFACD

SEQ ID NO: 85 - protein, 24 aa, synthetic construct
IYIWAPLAGTCGVLLLSLVITLYC

SEQ ID NO: 86 - protein, 42 aa, synthetic construct
KRGRKKLLYIFKQPFMRPVQTTQEEDGCSCRFPEEEEGGCEL

SEQ ID NO: 87 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 7 aa, synthetic construct
GFSLTSY

SEQ ID NO: 89 - protein, 10 aa, synthetic construct
GFSLTSYGVS

SEQ ID NO: 90 - protein, 5 aa, synthetic construct
SYGVS

SEQ ID NO: 91 - protein, 6 aa, synthetic construct
TSYGVS

SEQ ID NO: 92 - protein, 8 aa, synthetic construct
GFSLTSYG

SEQ ID NO: 93 - protein, 5 aa, synthetic construct
WEDGS

SEQ ID NO: 94 - protein, 9 aa, synthetic construct
VIWEDGSTN

SEQ ID NO: 95 - protein, 16 aa, synthetic construct
VIWEDGSTNYHSALIS

SEQ ID NO: 96 - protein, 12 aa, synthetic construct
WIGVIWEDGSTN

SEQ ID NO: 97 - protein, 7 aa, synthetic construct
IWEDGST

SEQ ID NO: 98 - protein, 13 aa, synthetic construct
PHYGSSYVGAMEY

SEQ ID NO: 99 - protein, 14 aa, synthetic construct
ARPHYGSSYVGAME

SEQ ID NO: 100 - protein, 15 aa, synthetic construct
ARPHYGSSYVGAMEY

SEQ ID NO: 101 - protein, 11 aa, synthetic construct
RASENIYSYVA

SEQ ID NO: 102 - protein, 7 aa, synthetic construct
YSYVAWY

SEQ ID NO: 103 - protein, 6 aa, synthetic construct
ENIYSY

SEQ ID NO: 104 - protein, 7 aa, synthetic construct
NAKSLAS

SEQ ID NO: 105 - protein, 10 aa, synthetic construct
LLIYNAKSLA

SEQ ID NO: 106 - skipped

SEQ ID NO: 107 - protein, 9 aa, synthetic construct
QHHYVSPWT

SEQ ID NO: 108 - protein, 8 aa, synthetic construct
QHHYVSPW

SEQ ID NO: 109 - protein, 107 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIK

SEQ ID NO: 110 - protein, 121 aa, synthetic construct
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 111 - protein, 785 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACWYFLIVLPVLLVIVIFAAVGVRSRRAQRQRQQRQRDRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 112 - protein, 778 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACWYFLIVLPVLLVIVIFAAVGVKRKKQRSRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 113 - protein, 780 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSKRRRSQRNRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 114 - protein, 776 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSKRKRTHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 115 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFGVISFIVILVVVVIILVGVVSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 116 - protein, 776 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACGLLLLLLLVTAALVALGVLYADKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 117 - protein, 777 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACWQVIWMMAAVMLVLTVVLGLYKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 118 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACWQVIWMMAAVMLVLTVVLGLYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 119 - protein, 779 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSRKRNKTGKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 120 - protein, 778 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 121 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACAIPLVLLVVLVFAGMGIFAAFKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 122 - protein, 777 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACVWVIAVIVIAIVVGVAVICVVPYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 123 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACKAVSGSIVLVIVLAGVGVLVWSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 124 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACVELAGGLGAILLLLVCLVTIYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 125 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACVVVLGMCTMFIVVCALVGVLVKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 126 - protein, 776 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACGAGLAGFFLGILAASLTLILIGKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 127 - protein, 779 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACGAIIGLMVGGVVIATVIVITLVMLRKKRKGKMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 128 - protein, 775 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACLLWAVLLLPAVALVLGFGVFVSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 129 - protein, 777 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACVVGGAGATALVFLSFCVIFVVKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 130 - protein, 778 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACWQVIWMMAAVMLVLTVVLGLYKRKKQRSRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 131 - protein, 813 aa, synthetic construct; identical to SEQ ID NO: 77

SEQ ID NO: 132 - protein, 813 aa, synthetic construct; identical to SEQ ID NO: 77

SEQ ID NO: 133 - protein, 5 aa, synthetic construct
GYTMN

SEQ ID NO: 134 - protein, 17 aa, synthetic construct
LITPYNGASSYNQKFRG

SEQ ID NO: 135 - protein, 10 aa, synthetic construct
GGYDGRGFDY

SEQ ID NO: 136 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 137 - protein, 10 aa, synthetic construct
SASSSVSYMH

SEQ ID NO: 138 - protein, 7 aa, synthetic construct
DTSKLAS

SEQ ID NO: 139 - protein, 9 aa, synthetic construct
QQWSGYPLT

SEQ ID NO: 140 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 141 - protein, 7 aa, synthetic construct
GYSFTGY

SEQ ID NO: 142 - protein, 10 aa, synthetic construct
GYSFTGYTMN

SEQ ID NO: 143 - protein, 6 aa, synthetic construct
TGYTMN

SEQ ID NO: 144 - protein, 8 aa, synthetic construct
GYSFTGYT

SEQ ID NO: 145 - protein, 6 aa, synthetic construct
TPYNGA

SEQ ID NO: 146 - protein, 10 aa, synthetic construct
LITPYNGASS

SEQ ID NO: 147 - protein, 13 aa, synthetic construct
WIGLITPYNGASS

SEQ ID NO: 148 - protein, 8 aa, synthetic construct
ITPYNGAS

SEQ ID NO: 149 - protein, 11 aa, synthetic construct
ARGGYDGRGFD

SEQ ID NO: 150 - protein, 12 aa, synthetic construct
ARGGYDGRGFDY

SEQ ID NO: 151 - protein, 6 aa, synthetic construct
SYMHWY

SEQ ID NO: 152 - protein, 5 aa, synthetic construct
SSVSY

SEQ ID NO: 153 - protein, 10 aa, synthetic construct
RWIYDTSKLA

SEQ ID NO: 154 - protein, 8 aa, synthetic construct
QQWSGYPL

SEQ ID NO: 155 - protein, 7 aa, synthetic construct
ENLYTQS

SEQ ID NO: 156 - protein, 5 aa, synthetic construct
DDDDK

SEQ ID NO: 157 - protein, 4 aa, synthetic construct
LVPR

SEQ ID NO: 158 - protein, 4 aa, synthetic construct
CPPC

SEQ ID NO: 159 - RNA, 103 nt, synthetic construct
  source 1..103: mol_type = other RNA
ggggccactagggacaggatgttttagagctagaaatagcaagttaaaataaggctagtccgttatcaacttgaaaaagtggcaccgagtcggtgcttttttt

SEQ ID NO: 160 - DNA, 20 bp, Adeno-associated virus
  source 1..20: mol_type = other DNA
ggggccactagggacaggat

SEQ ID NO: 161 - RNA, 20 nt, synthetic construct
  source 1..20: mol_type = other RNA
gcacctgaataccacgcctg

SEQ ID NO: 162 - RNA, 20 nt, synthetic construct
  source 1..20: mol_type = other RNA
cgcctgcgatgtagtcgatg

SEQ ID NO: 163 - RNA, 20 nt, synthetic construct
  source 1..20: mol_type = other RNA
caggacgggcgagatgtccc

SEQ ID NO: 164 - RNA, 20 nt, synthetic construct
  source 1..20: mol_type = other RNA
ctgaatctttggagtacctg

SEQ ID NO: 165 - RNA, 20 nt, synthetic construct
  source 1..20: mol_type = other RNA
ggccacggagcgagacatct

SEQ ID NO: 166 - RNA, 20 nt, synthetic construct
  source 1..20: mol_type = other RNA
aagtcaacttcaatgtcgga

SEQ ID NO: 167 - RNA, 20 nt, synthetic construct
  source 1..20: mol_type = other RNA
gcttggaggcctgatcagcg

SEQ ID NO: 168 - RNA, 20 nt, synthetic construct
  source 1..20: mol_type = other RNA
cttatctcttcgcagcgagg

SEQ ID NO: 169 - RNA, 20 nt, synthetic construct
  source 1..20: mol_type = other RNA
cacacattactccaacattg

SEQ ID NO: 170 - RNA, 20 nt, synthetic construct
  source 1..20: mol_type = other RNA
ttccgcaaaatagagcccca

SEQ ID NO: 171 - RNA, 20 nt, synthetic construct
  source 1..20: mol_type = other RNA
tgcacagaactatcgtacca

SEQ ID NO: 172 - RNA, 20 nt, synthetic construct
  source 1..20: mol_type = other RNA
gcaataagactctttaaaga

SEQ ID NO: 173 - RNA, 20 nt, synthetic construct
  source 1..20: mol_type = other RNA
caaagagattacgaatgcct

SEQ ID NO: 174 - RNA, 20 nt, synthetic construct
  source 1..20: mol_type = other RNA
caaggcaccccaggtttcca

SEQ ID NO: 175 - RNA, 20 nt, synthetic construct
  source 1..20: mol_type = other RNA
ttacgaatgccttggaaacc

SEQ ID NO: 176 - RNA, 20 nt, synthetic construct
  source 1..20: mol_type = other RNA
cagagacgcatctgaccctc

SEQ ID NO: 177 - RNA, 20 nt, synthetic construct
  source 1..20: mol_type = other RNA
catgcagttctcacacactg

SEQ ID NO: 178 - RNA, 20 nt, synthetic construct
  source 1..20: mol_type = other RNA
gtgtgagaactgcatggaga

SEQ ID NO: 179 - RNA, 20 nt, synthetic construct
  source 1..20: mol_type = other RNA
tctcatttcaggaaaccact

SEQ ID NO: 180 - RNA, 20 nt, synthetic construct
  source 1..20: mol_type = other RNA
agtcatacaccttaaccaag

SEQ ID NO: 181 - RNA, 20 nt, synthetic construct
  source 1..20: mol_type = other RNA
ttcaaggaaaccagttgagg

SEQ ID NO: 182 - RNA, 20 nt, synthetic construct
  source 1..20: mol_type = other RNA
gagccttgcctggaaatctg

SEQ ID NO: 183 - RNA, 20 nt, synthetic construct
  source 1..20: mol_type = other RNA
aagcgtcaaaagtctgccag

SEQ ID NO: 184 - RNA, 20 nt, synthetic construct
  source 1..20: mol_type = other RNA
cgttccaactcgaagtgcca

SEQ ID NO: 185 - RNA, 20 nt, synthetic construct
  source 1..20: mol_type = other RNA
gagcgactgggacacggtga

SEQ ID NO: 186 - RNA, 20 nt, synthetic construct
  source 1..20: mol_type = other RNA
gctgcgcaagaagggcccta

SEQ ID NO: 187 - RNA, 20 nt, synthetic construct
  source 1..20: mol_type = other RNA
ttgttctggccagcagcccc

SEQ ID NO: 188 - RNA, 20 nt, synthetic construct
  source 1..20: mol_type = other RNA
cttccagagccacatcatcg

SEQ ID NO: 189 - RNA, 20 nt, synthetic construct
  source 1..20: mol_type = other RNA
gggactcaccagagagaggt

SEQ ID NO: 190 - RNA, 20 nt, synthetic construct
  source 1..20: mol_type = other RNA
cggtcgaaatagaagcccta

SEQ ID NO: 191 - RNA, 20 nt, synthetic construct
  source 1..20: mol_type = other RNA
aaaaggatattgtgcaactg

SEQ ID NO: 192 - RNA, 20 nt, synthetic construct
  source 1..20: mol_type = other RNA
tgtgcatatttattacatcg

SEQ ID NO: 193 - RNA, 20 nt, synthetic construct
  source 1..20: mol_type = other RNA
tttgtgaagatcttgaccaa

SEQ ID NO: 194 - RNA, 20 nt, synthetic construct
  source 1..20: mol_type = other RNA
tgtcatgctgaaccgcattg

SEQ ID NO: 195 - RNA, 20 nt, synthetic construct
  source 1..20: mol_type = other RNA
ccactctatgaggatagtca

SEQ ID NO: 196 - RNA, 20 nt, synthetic construct
  source 1..20: mol_type = other RNA
ttgacatagaagaggcacaa

SEQ ID NO: 197 - RNA, 20 nt, synthetic construct
  source 1..20: mol_type = other RNA
gagtactacactcagcagca

SEQ ID NO: 198 - RNA, 20 nt, synthetic construct
  source 1..20: mol_type = other RNA
tcacgcacaagaaacgtcca

SEQ ID NO: 199 - RNA, 20 nt, synthetic construct
  source 1..20: mol_type = other RNA
aggtctcggtgaaaccacct

SEQ ID NO: 200 - RNA, 20 nt, synthetic construct
  source 1..20: mol_type = other RNA
agcattatccaaagagtccg

SEQ ID NO: 201 - RNA, 20 nt, synthetic construct
  source 1..20: mol_type = other RNA
atattaattcttaccagtgg

SEQ ID NO: 202 - RNA, 20 nt, synthetic construct
  source 1..20: mol_type = other RNA
agctttaaatcaaggttcat

SEQ ID NO: 203 - RNA, 20 nt, synthetic construct
  source 1..20: mol_type = other RNA
atcccgagccctaaggtgca

SEQ ID NO: 204 - RNA, 20 nt, synthetic construct
  source 1..20: mol_type = other RNA
ggcagcgcggaggacagcgt

SEQ ID NO: 205 - RNA, 20 nt, synthetic construct
  source 1..20: mol_type = other RNA
ctcagggggctactaccacc

SEQ ID NO: 206 - RNA, 20 nt, synthetic construct
  source 1..20: mol_type = other RNA
gtcaccgacgagaccagaag

SEQ ID NO: 207 - RNA, 20 nt, synthetic construct
  source 1..20: mol_type = other RNA
gtcgtggacttcgtactgct

SEQ ID NO: 208 - RNA, 20 nt, synthetic construct
  source 1..20: mol_type = other RNA
taatttttaggcaagtgtcg

SEQ ID NO: 209 - RNA, 20 nt, synthetic construct
  source 1..20: mol_type = other RNA
ttagctgttagacttgaata

SEQ ID NO: 210 - RNA, 20 nt, synthetic construct
  source 1..20: mol_type = other RNA
cgagagccgtcaacttgcgt

SEQ ID NO: 211 - RNA, 20 nt, synthetic construct
  source 1..20: mol_type = other RNA
cggcttcaactgcaaaggtg

SEQ ID NO: 212 - RNA, 20 nt, synthetic construct
  source 1..20: mol_type = other RNA
tatgaaaaagcagagcgact

SEQ ID NO: 213 - RNA, 20 nt, synthetic construct
  source 1..20: mol_type = other RNA
tctggcgggcgagctcacgc

SEQ ID NO: 214 - RNA, 20 nt, synthetic construct
  source 1..20: mol_type = other RNA
ctcacgctggttaccgccta

SEQ ID NO: 215 - RNA, 20 nt, synthetic construct
  source 1..20: mol_type = other RNA
aaagattacgaacttccctg

SEQ ID NO: 216 - RNA, 20 nt, synthetic construct
  source 1..20: mol_type = other RNA
gttaaaaacagacatgccta

SEQ ID NO: 217 - RNA, 20 nt, synthetic construct
  source 1..20: mol_type = other RNA
atgcctaaggaggttgtacc

SEQ ID NO: 218 - RNA, 20 nt, synthetic construct
  source 1..20: mol_type = other RNA
ctccaggtatcccatcgaaa

SEQ ID NO: 219 - RNA, 20 nt, synthetic construct
  source 1..20: mol_type = other RNA
caccaaatacgatagatcag

SEQ ID NO: 220 - RNA, 20 nt, synthetic construct
  source 1..20: mol_type = other RNA
tggcggcgtgaatggcaaga

SEQ ID NO: 221 - RNA, 20 nt, synthetic construct
  source 1..20: mol_type = other RNA
taggatggtagcacacaacc

SEQ ID NO: 222 - RNA, 20 nt, synthetic construct
  source 1..20: mol_type = other RNA
cagcagcagagccccgacgg

SEQ ID NO: 223 - RNA, 20 nt, synthetic construct
  source 1..20: mol_type = other RNA
cggcgtgcgaacggaatgtg

SEQ ID NO: 224 - RNA, 20 nt, synthetic construct
  source 1..20: mol_type = other RNA
tatagacgctgcccgacgtc

SEQ ID NO: 225 - RNA, 20 nt, synthetic construct
  source 1..20: mol_type = other RNA
tccaaagaagggtactgtgg

SEQ ID NO: 226 - RNA, 20 nt, synthetic construct
  source 1..20: mol_type = other RNA
acagtacccttctttggaat

SEQ ID NO: 227 - RNA, 20 nt, synthetic construct
  source 1..20: mol_type = other RNA
gcgacgggcgcatctacgtg

SEQ ID NO: 228 - RNA, 20 nt, synthetic construct
  source 1..20: mol_type = other RNA
cccgacctccataagtcctg

SEQ ID NO: 229 - RNA, 20 nt, synthetic construct
  source 1..20: mol_type = other RNA
ggggtcctcgaagcgcacga

SEQ ID NO: 230 - RNA, 20 nt, synthetic construct
  source 1..20: mol_type = other RNA
tgctctgtttagaagatgac

SEQ ID NO: 231 - RNA, 20 nt, synthetic construct
  source 1..20: mol_type = other RNA
atattcttttctagttaaag

SEQ ID NO: 232 - RNA, 20 nt, synthetic construct
  source 1..20: mol_type = other RNA
cctgtaaagaaacaaaagac

SEQ ID NO: 233 - RNA, 20 nt, synthetic construct
  source 1..20: mol_type = other RNA
tggagaaagacgtaacttcg

SEQ ID NO: 234 - RNA, 20 nt, synthetic construct
  source 1..20: mol_type = other RNA
tctgccctgaggtatgcgat

SEQ ID NO: 235 - RNA, 20 nt, synthetic construct
  source 1..20: mol_type = other RNA
attccgcttggtgaaaacga

SEQ ID NO: 236 - RNA, 20 nt, synthetic construct
  source 1..20: mol_type = other RNA
caggcacaatagaaacaacg

SEQ ID NO: 237 - RNA, 20 nt, synthetic construct
  source 1..20: mol_type = other RNA
ccatttgtaatgctgacttg

SEQ ID NO: 238 - RNA, 20 nt, synthetic construct
  source 1..20: mol_type = other RNA
ctgggtcacttgtgccgtgg

SEQ ID NO: 239 - RNA, 20 nt, synthetic construct
  source 1..20: mol_type = other RNA
gtcagggttctggatatctg

SEQ ID NO: 240 - RNA, 20 nt, synthetic construct
  source 1..20: mol_type = other RNA
tggatttagagtctctcagc

SEQ ID NO: 241 - RNA, 20 nt, synthetic construct
  source 1..20: mol_type = other RNA
ctgcggctgtggtccagctg

SEQ ID NO: 242 - RNA, 20 nt, synthetic construct
  source 1..20: mol_type = other RNA
acaaaactgtgctagacatg

SEQ ID NO: 243 - RNA, 20 nt, synthetic construct
  source 1..20: mol_type = other RNA
ttcttccccagcccaggtaa

SEQ ID NO: 244 - RNA, 20 nt, synthetic construct
  source 1..20: mol_type = other RNA
cgtcatgagcagattaaacc

SEQ ID NO: 245 - RNA, 20 nt, synthetic construct
  source 1..20: mol_type = other RNA
gagagcgcctgcgacccgag

SEQ ID NO: 246 - RNA, 20 nt, synthetic construct
  source 1..20: mol_type = other RNA
ccagcgggtgaagtacacca

SEQ ID NO: 247 - RNA, 20 nt, synthetic construct
  source 1..20: mol_type = other RNA
ggagcgcttttcgccgccag

SEQ ID NO: 248 - RNA, 20 nt, synthetic construct
  source 1..20: mol_type = other RNA
tgaggcctggaccttatgca

SEQ ID NO: 249 - RNA, 20 nt, synthetic construct
  source 1..20: mol_type = other RNA
cctggtggagtgaaccatga

SEQ ID NO: 250 - RNA, 20 nt, synthetic construct
  source 1..20: mol_type = other RNA
caagcacttaggttcccctg

SEQ ID NO: 251 - RNA, 20 nt, synthetic construct
  source 1..20: mol_type = other RNA
ggtctccctacaattcagcg

SEQ ID NO: 252 - RNA, 20 nt, synthetic construct
  source 1..20: mol_type = other RNA
cacagcgcgtgactgcaatg

SEQ ID NO: 253 - RNA, 20 nt, synthetic construct
  source 1..20: mol_type = other RNA
tctggggcaccaattctagg

SEQ ID NO: 254 - RNA, 20 nt, synthetic construct
  source 1..20: mol_type = other RNA
gagccatgcttggcttacga

SEQ ID NO: 255 - RNA, 20 nt, synthetic construct
  source 1..20: mol_type = other RNA
gtacaagtacttatctcatg

SEQ ID NO: 256 - RNA, 20 nt, synthetic construct
  source 1..20: mol_type = other RNA
gagataacaacataacaaca

SEQ ID NO: 257 - RNA, 20 nt, synthetic construct
  source 1..20: mol_type = other RNA
catattccatagtctttggg

SEQ ID NO: 258 - RNA, 20 nt, synthetic construct
  source 1..20: mol_type = other RNA
ctgccccttagcaacttagg

SEQ ID NO: 259 - RNA, 20 nt, synthetic construct
  source 1..20: mol_type = other RNA
tgtttaaaaatatgttgaca

SEQ ID NO: 260 - RNA, 20 nt, synthetic construct
  source 1..20: mol_type = other RNA
ccaggaatggaaactcacgc

SEQ ID NO: 261 - RNA, 20 nt, synthetic construct
  source 1..20: mol_type = other RNA
gaggccgctgaattaacccg

SEQ ID NO: 262 - RNA, 20 nt, synthetic construct
  source 1..20: mol_type = other RNA
atacacgcacacttgcagaa

SEQ ID NO: 263 - RNA, 20 nt, synthetic construct
  source 1..20: mol_type = other RNA
gagcagacagaaacccaggg

SEQ ID NO: 264 - RNA, 20 nt, synthetic construct
  source 1..20: mol_type = other RNA
tgagtctccaaacagaacag

SEQ ID NO: 265 - RNA, 20 nt, synthetic construct
  source 1..20: mol_type = other RNA
taatatcactgacttcacgg

SEQ ID NO: 266 - RNA, 20 nt, synthetic construct
  source 1..20: mol_type = other RNA
tacacacaatgtaagcagca

SEQ ID NO: 267 - RNA, 20 nt, synthetic construct
  source 1..20: mol_type = other RNA
gggagctcaattcgaaacca

SEQ ID NO: 268 - RNA, 20 nt, synthetic construct
  source 1..20: mol_type = other RNA
ttggacaggtgagacagtcg

SEQ ID NO: 269 - RNA, 20 nt, synthetic construct
  source 1..20: mol_type = other RNA
aagctcactcagatagtgtg

SEQ ID NO: 270 - RNA, 20 nt, synthetic construct
  source 1..20: mol_type = other RNA
caggagaaccaccttacacg

SEQ ID NO: 271 - RNA, 20 nt, synthetic construct
  source 1..20: mol_type = other RNA
ggacagaccctgattcacaa

SEQ ID NO: 272 - RNA, 20 nt, synthetic construct
  source 1..20: mol_type = other RNA
acatggcagtctatgaacag

SEQ ID NO: 273 - RNA, 20 nt, synthetic construct
  source 1..20: mol_type = other RNA
cctatagagagtactacttg

SEQ ID NO: 274 - RNA, 20 nt, synthetic construct
  source 1..20: mol_type = other RNA
ccaaccgggtcttcattacg

SEQ ID NO: 275 - RNA, 20 nt, synthetic construct
  source 1..20: mol_type = other RNA
tcaagcgtagagttccgagt

SEQ ID NO: 276 - RNA, 20 nt, synthetic construct
  source 1..20: mol_type = other RNA
tcatgcaattatggacccag

SEQ ID NO: 277 - RNA, 20 nt, synthetic construct
  source 1..20: mol_type = other RNA
cgggaaagtgactggccatg

SEQ ID NO: 278 - RNA, 20 nt, synthetic construct
  source 1..20: mol_type = other RNA
tgagattgaaatcaaatcgg

SEQ ID NO: 279 - RNA, 20 nt, synthetic construct
  source 1..20: mol_type = other RNA
tatgcaatattcatcacgcg

SEQ ID NO: 280 - RNA, 20 nt, synthetic construct
  source 1..20: mol_type = other RNA
aatgtgttaaatcaaatgca

SEQ ID NO: 281 - protein, 541 aa, synthetic construct
TTTPAPRPPTPAPTIASQPLSLRPEACWYFLIVLPVLLVIVIFAAVGVRSRRAQRQRQQRQRDRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 282 - protein, 534 aa, synthetic construct
TTTPAPRPPTPAPTIASQPLSLRPEACWYFLIVLPVLLVIVIFAAVGVKRKKQRSRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 283 - protein, 536 aa, synthetic construct
TTTPAPRPPTPAPTIASQPLSLRPEACFMYVAAAAFVLLFFVGCGVLLSKRRRSQRNRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 284 - protein, 532 aa, synthetic construct
TTTPAPRPPTPAPTIASQPLSLRPEACFMYVAAAAFVLLFFVGCGVLLSKRKRTHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 285 - protein, 531 aa, synthetic construct
TTTPAPRPPTPAPTIASQPLSLRPEACFGVISFIVILVVVVIILVGVVSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 286 - protein, 532 aa, synthetic construct
TTTPAPRPPTPAPTIASQPLSLRPEACGLLLLLLLVTAALVALGVLYADKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 287 - protein, 533 aa, synthetic construct
TTTPAPRPPTPAPTIASQPLSLRPEACWQVIWMMAAVMLVLTVVLGLYKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 288 - protein, 531 aa, synthetic construct
TTTPAPRPPTPAPTIASQPLSLRPEACWQVIWMMAAVMLVLTVVLGLYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 289 - protein, 535 aa, synthetic construct
TTTPAPRPPTPAPTIASQPLSLRPEACFMYVAAAAFVLLFFVGCGVLLSRKRNKTGKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 290 - protein, 534 aa, synthetic construct
TTTPAPRPPTPAPTIASQPLSLRPEACFMYVAAAAFVLLFFVGCGVLLSKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 291 - protein, 531 aa, synthetic construct
TTTPAPRPPTPAPTIASQPLSLRPEACAIPLVLLVVLVFAGMGIFAAFKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 292 - protein, 533 aa, synthetic construct
TTTPAPRPPTPAPTIASQPLSLRPEACVWVIAVIVIAIVVGVAVICVVPYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 293 - protein, 531 aa, synthetic construct
TTTPAPRPPTPAPTIASQPLSLRPEACKAVSGSIVLVIVLAGVGVLVWSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 294 - protein, 531 aa, synthetic construct
TTTPAPRPPTPAPTIASQPLSLRPEACVELAGGLGAILLLLVCLVTIYSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 295 - protein, 531 aa, synthetic construct
TTTPAPRPPTPAPTIASQPLSLRPEACVVVLGMCTMFIVVCALVGVLVKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 296 - protein, 532 aa, synthetic construct
TTTPAPRPPTPAPTIASQPLSLRPEACGAGLAGFFLGILAASLTLILIGKRKRKHMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 297 - protein, 535 aa, synthetic construct
TTTPAPRPPTPAPTIASQPLSLRPEACGAIIGLMVGGVVIATVIVITLVMLRKKRKGKMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 298 - protein, 531 aa, synthetic construct
TTTPAPRPPTPAPTIASQPLSLRPEACLLWAVLLLPAVALVLGFGVFVSRKRRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 299 - protein, 533 aa, synthetic construct
TTTPAPRPPTPAPTIASQPLSLRPEACVVGGAGATALVFLSFCVIFVVKKRRQRQRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 300 - protein, 534 aa, synthetic construct
TTTPAPRPPTPAPTIASQPLSLRPEACWQVIWMMAAVMLVLTVVLGLYKRKKQRSRRMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 301 - protein, 569 aa, synthetic construct
TTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 302 - protein, 569 aa, synthetic construct; identical to SEQ ID NO: 301

(19) United States
(12) Patent Application Publication
(10) Pub. No.: US 2026/0130938 A1
(43) Pub. Date: May 14, 2026

(54) NOVEL RECEPTORS FOR TRANSCRIPTION REGULATION

(71) Applicant: Arsenal Biosciences, Inc., South San Francisco, CA (US)

(72) Inventors: Nishant Mehta, San Mateo, CA (US); Aaron Cooper, Berkeley, CA (US); Jonah Pierce, San Francisco, CA (US); Sofia Kyriazopoulou Panagiotopoulou, South San Francisco, CA (US)

(21) Appl. No.: 19/360,889

(22) Filed: Oct. 16, 2025

Related U.S. Application Data
(63) Continuation of application No. PCT/US24/26307, filed on Apr. 25, 2024.
(60) Provisional application No. 63/498,132, filed on Apr. 25, 2023.

(51) Int. Cl. A61K 35/17; A61K 40/11; A61K 40/31; A61K 40/42; A61P 35/00; C07K 14/705; C07K 14/725; C07K 16/18; C07K 16/40; C12N 9/22; C12N 15/11; C12N 15/85; C12N 15/90

(52) U.S. Cl. CPC A61K 35/17; A61K 40/11; A61K 40/31; A61K 40/4244; A61P 35/00; C07K 14/7051; C07K 14/70517; C07K 14/70578; C07K 16/18; C07K 16/40; C12N 9/226; C12N 15/111; C12N 15/85; C12N 15/907; A61K 2239/13; A61K 2239/17; A61K 2239/21; A61K 2239/22; C07K 2317/622; C07K 2319/71; C12N 2310/20; C12N 2830/002; C12N 2830/48

(57) ABSTRACT

Provided herein are chimeric priming receptors with novel transmembrane domains and novel juxtamembrane domains. Also provided are systems of chimeric priming receptors with novel transmembrane domains and novel juxtamembrane domains, cells expressing such receptors and systems, and methods of use thereof.

### CROSS REFERENCE TO RELATED APPLICATIONS

This application is a continuation of International Application Number PCT/US2024/026307, filed Apr. 25, 2024, which claims the benefit of U.S. Provisional Application No. 63/498,132, filed Apr. 25, 2023, each which is hereby incorporated in its entirety by reference.

### SEQUENCE LISTING

The instant application contains a Sequence Listing which is hereby incorporated by reference in its entirety. Said XML copy, created on Oct. 16, 2025, is named ANB-208USWOC1_SL.xml, and is 297,939 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them.

After the T cells are engineered with the CARs, the resulting CAR-T cells are introduced into patients to attack tumor cells. CAR-T cells can be either derived from T cells in a patient's own blood (autologous) or derived from the T cells of another healthy donor (allogeneic). Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some concerns and limitations to CAR T cell-based immunotherapy. Some CAR T cells may engage with normal cells expressing low levels of target antigens, leading to off target toxicity. Synthetic AND-gate circuits in T cells can operate by binding to a first antigen and inducing the expression of a second receptor that responds to a second antigen. Depending on the binding affinity, basal activity, or expression level of the second receptor, higher or lower levels of “inducibility” of the second protein may be required for optimal function. Tight control of this induction is necessary to prevent the expression of the second protein without the presence of the “priming” antigen. Even low amounts of basal induction could lead to increased toxicity risk. Thus, additional priming receptors are required that reduce off-target toxicity.

### SUMMARY

In one aspect, provided herein are priming receptors comprising, in an N-terminus to C-terminus direction: optionally, an extracellular antigen-binding domain having a binding affinity for an antigen; an STS, SNORC, SEM4F, MCP, PLAT2, MMEL1, PLXC1, CSTN2, SIGL9, IRAG1, TIGIT, TNR21, ZP3, SUSD4, DCBD1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites.

In one aspect, provided herein are priming receptors comprising, in an N-terminus to C-terminus direction: an extracellular antigen-binding domain having a binding affinity for Alkaline Phosphatase, Placental/Germ Cell (ALPG/P); an STS, SNORC, SEM4F, MCP, PLAT2, MMEL1, PLXC1, CSTN2, SIGL9, IRAG1, TIGIT, TNR21, ZP3, SUSD4, DCBD1, IRAG1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites.

In one aspect, provided herein are priming receptors comprising, in an N-terminus to C-terminus direction: an extracellular antigen-binding domain having a binding affinity for CD19; mesothelin (MSLN); Alkaline Phosphatase, Placental/Germ Cell (ALPG/P); epidermal growth factor receptor (EGFR); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1); an STS, SNORC, SEM4F, MCP, PLAT2, MMEL1, PLXC1, CSTN2, SIGL9, IRAG1, TIGIT, TNR21, ZP3, SUSD4, DCBD1, IRAG1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites.

In some embodiments, the priming receptor further comprises a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JMD).

In one aspect, provided herein are priming receptors comprising, in an N-terminus to C-terminus direction: optionally, an extracellular antigen-binding domain having a binding affinity for an antigen; a transmembrane domain comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, or TMIG3 juxtamembrane domain (JMD); and an intracellular domain comprising a human or humanized transcriptional effector, wherein binding of the antigen to the extracellular antigen-binding domain results in cleavage at the one or more ligand-inducible proteolytic cleavage sites.

In one aspect, provided herein are priming receptors comprising, in an N-terminus to C-terminus direction: an extracellular antigen-binding domain having a binding affinity for Alkaline Phosphatase, Placental/Germ Cell (ALPG/P); a transmembrane domain comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, or TMIG3, juxtamembrane domain (JMD); and an intracellular domain comprising a human or humanized transcriptional effector, wherein binding of the antigen to the extracellular antigen-binding domain results in cleavage at the one or more ligand-inducible proteolytic cleavage sites.

In some embodiments, the priming receptor further comprises an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, IRAG1, HMR1, DISP1, IRPL1, ECSCR, PLXC1, or NOTCH1 transmembrane domain comprising one or more antigen-inducible proteolytic cleavage sites.

In one aspect, provided herein are priming receptors comprising, in an N-terminus to C-terminus direction: optionally, an extracellular antigen-binding domain having a binding affinity for an antigen; an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1, or NOTCH1 transmembrane domain comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JMD); and an intracellular domain comprising a human or humanized transcriptional effector, wherein binding of the antigen to the extracellular antigen-binding domain results in cleavage at the one or more ligand-inducible proteolytic cleavage sites.

In one aspect, provided herein are priming receptors comprising, in an N-terminus to C-terminus direction: an extracellular antigen-binding domain having a binding affinity for Alkaline Phosphatase, Placental/Germ Cell (ALPG/P); an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1, or NOTCH1 transmembrane domain (TMD) comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JMD); and an intracellular domain comprising a human or humanized transcriptional effector, wherein binding of the antigen to the extracellular antigen-binding domain results in cleavage at the ligand-inducible proteolytic cleavage site.

In some embodiments, the transmembrane domain comprises a sequence set forth in at least one of SEQ ID NOs: 1-21.

In some embodiments, the juxtamembrane domain comprises a sequence set forth in at least one of SEQ ID NOs: 22-39.

In some embodiments, the transmembrane domain and juxtamembrane domain comprise a combination of at least one of: NOTCH1-PLXA2, CLSTN1-CD2, NOTCH1-NPTN, NOTCH1-ALK, SNORC-NOTCH1, SEM4F-NOTCH2, STS-NOTCH2, MCP-NOTCH1, DCBD1-NOTCH2, IRAG1-C163A, HMR1-NOTCH1, MCP-NOTCH1, DISP1-NOTCH2, IRPL1-NOTCH1, ECSCR-NOTCH1, NOTCH1-PTPRF, NOTCH1-CSPG4, SEM4F-NOTCH2, IRAG1-CD2, SIGL9-CD2, IRAG1-NOTCH1, NOTCH1-SLAF1, NOTCH1-TMIG3, NOTCH1-C163A, and PLXC1-EPHA1.

In some embodiments, the transmembrane domain and juxtamembrane domain comprise a sequence set forth in at least one of SEQ ID NOs: 40-56.

In some embodiments, the extracellular antigen-binding domain is a monoclonal antibody, a neutral antibody, an antagonistic antibody, an agonist antibody, a polyclonal antibody, an afucosylated antibody, a human antibody, a humanized antibody, a chimeric antibody, a full-length antibody, and an scFv.

In some embodiments, the extracellular antigen-binding domain is an scFv.

In some embodiments, the ALPG/P extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 88, 89, 90, 91 or 92, CDR-H2 comprises the sequence set forth in SEQ ID NO: 93, 94, 95, 96, or 97, CDR-H3 comprises the sequence set forth in SEQ ID NO: 98, 99 or 100, CDR-L1 comprises the sequence set forth in SEQ ID NO: 101, 102, or 103, CDR-L2 comprises the sequence set forth in SEQ ID NO: 104, 105, or “NA”; and CDR-L3 comprises the sequence set forth in SEQ ID NO: 107 or 108.

In some embodiments, the VH chain sequence comprises the sequence set forth in SEQ ID NO: 110.

In some embodiments, the VL comprises the sequence set forth in SEQ ID NO: 109.

In some embodiments, the extracellular domain comprises the sequence set forth in SEQ ID NO: 76.

In some embodiments, the transcriptional effector domain comprises an HNF1a/p65 domain or a Gal4/VP64 domain.

In some embodiments, the transcriptional effector domain comprises the sequence as set forth in SEQ ID NO: 80, 81, or 82.

In some embodiments, the priming receptor further comprises a first hinge domain positioned between the first extracellular antigen-binding domain and the first transmembrane domain.

In some embodiments, the hinge domain comprises a CD8α hinge domain or a truncated CD8α hinge domain.

In some embodiments, the priming receptor comprises a sequence as set forth in at least one of SEQ ID NO: 281-300.

In some embodiments, the priming receptor comprises a sequence as set forth in at least one of SEQ ID NO: 111-132.

In some embodiments, the priming receptor induces expression of a chimeric antigen receptor (CAR) after binding of the extracellular antigen-binding domain to the antigen.

In some embodiments, the chimeric antigen receptor (CAR) comprises an extracellular antigen-binding domain having a binding affinity for mesothelin (MSLN).

In one aspect, provided herein are systems comprising a first chimeric polypeptide and a second chimeric polypeptide, wherein the first chimeric polypeptide comprises the priming receptor disclosed herein; and the second chimeric polypeptide comprises a chimeric antigen receptor (CAR) comprising a second extracellular antigen-binding domain.

In some embodiments, the CAR comprises, from N-terminus to C-terminus: the second extracellular antigen-binding domain; a second transmembrane domain; optionally an intracellular co-stimulatory domain; and an intracellular activation domain.

In some embodiments, the CAR comprises an extracellular antigen-binding domain having a binding affinity for mesothelin (MSLN).

In some embodiments, the CAR comprises, from N-terminus to C-terminus: the second extracellular antigen-binding domain having a binding affinity for MSLN; a second transmembrane domain; an intracellular co-stimulatory domain; and an intracellular activation domain.

In some embodiments, the CAR comprises a second hinge domain.

In some embodiments, the second hinge domain comprises a CD8α hinge domain or a truncated CD8α hinge domain.

In some embodiments, the second transmembrane domain comprises a CD8α transmembrane domain.

In some embodiments, the intracellular co-stimulatory domain comprises a 4-1BB domain.

In some embodiments, the intracellular activation domain comprises a CD3ζ domain.

In some embodiments, the system further comprises a self-excising 2A peptide (P2A).

In some embodiments, the P2A is at the C-terminus of the chimeric antigen receptor.

In some embodiments, the P2A is at the C-terminus of the priming receptor.

In some embodiments, the first extracellular domain comprises an ALPG/P antigen-binding domain capable of binding to a target cell and the second extracellular domain comprises an MSLN antigen-binding domain capable of binding to the target cell.

In some embodiments, the second extracellular domain comprises a CD19; mesothelin (MSLN); Alkaline Phosphatase, Placental/Germ Cell (ALPG/P); epidermal growth factor receptor (EGFR); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1) antigen-binding domain capable of binding to the target cell.

In some embodiments, the target cell is a human cell.

In some embodiments, the target cell is a cancer cell.

In some embodiments, the cancer is a solid cancer or a liquid cancer.

In some embodiments, the cancer is ovarian cancer, fallopian cancer, primary peritoneal cancer, uterine cancer, mesothelioma, cervical cancer, or pancreatic cancer.

In one aspect, provided herein are recombinant nucleic acid systems comprising at least one nucleotide sequence encoding the priming receptor disclosed herein.

In one aspect, provided herein are recombinant nucleic acid systems comprising at least one nucleotide sequence encoding the system disclosed herein.

In some embodiments, the recombinant nucleic acid system comprises an inducible promoter operably linked to the nucleotide sequence encoding the chimeric antigen receptor.

In some embodiments, the recombinant nucleic acid system further comprises a constitutive promoter operably linked to the nucleotide sequence encoding the priming receptor.

In some embodiments, the recombinant nucleic acid system further comprises an inducible promoter operably linked to the nucleotide sequence encoding the chimeric antigen receptor and a constitutive promoter operably linked to the nucleotide sequence encoding the priming receptor.

In some embodiments, the nucleic acid system comprises, in a 5′ to 3′ direction, the constitutive promoter; the nucleotide sequence encoding the priming receptor; the inducible promoter; and the nucleotide sequence encoding the chimeric antigen receptor.

In some embodiments, the nucleic acid comprises, in a 5′ to 3′ direction, the inducible promoter; the nucleotide sequence encoding the chimeric antigen receptor; the constitutive promoter; and the nucleotide sequence encoding the priming receptor.

In some embodiments, the nucleic acid system further comprises a 5′ homology directed repair arm and a 3′ homology directed repair arm complementary to an insertion site in a host cell chromosome.

In some embodiments, the recombinant nucleic acid system further comprises a self-excising 2A peptide (P2A).

In some embodiments, the P2A is at the 3′ end of the chimeric antigen receptor.

In some embodiments, the P2A is at the 3′ end of the priming receptor.

In some embodiments, the recombinant nucleic acid system further comprises a woodchuck hepatitis virus post-translational regulatory element (WPRE).

In some embodiments, the WPRE is at the 3′ end of the nucleotide sequence encoding the chimeric antigen receptor and at the 5′ end of the nucleotide sequence encoding the priming receptor or wherein the WPRE is at the 3′ end of the nucleotide sequence encoding the priming receptor and at the 5′ end of the nucleotide sequence encoding the chimeric antigen receptor.

In some embodiments, the nucleic acid system is incorporated into an expression cassette and/or an expression vector.

In some embodiments, the expression vector is a non-viral vector.

In one aspect, provided herein are non-viral vectors comprising the recombinant nucleic acid system disclosed herein.

In some embodiments, the 5′ and 3′ ends of the recombinant nucleic acid system comprise nucleotide sequences that are homologous to genomic sequences flanking an insertion site in a genome of a cell, optionally a primary cell.

In some embodiments, the insertion site is at a T Cell Receptor Alpha Constant (TRAC) locus or a genomic safe harbor (GSH) locus.

In one aspect, provided herein are immune cells comprising: the priming receptor disclosed herein; the system disclosed herein; a recombinant nucleic acid system disclosed herein; and/or the non-viral vector disclosed herein.

In some embodiments, the immune cell is a primary human immune cell.

In some embodiments, the primary immune cell is an autologous immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In some embodiments, the primary immune cell is virus-free or does not comprise a viral vector for introducing the recombinant nucleic acid system.

In some embodiments, the cell further comprises obtaining the immune cell from a patient and introducing the system, the recombinant nucleic acid system and/or the non-viral vector in vitro.

In one aspect, provided herein are primary immune cells comprising at least one recombinant nucleic acid comprising a priming receptor comprising an extracellular antigen-binding domain, an STS, SNORC, SEM4F, MCP, PLAT2, MMEL1, PLXC1, CSTN2, SIGL9, IRAG1, TIGIT, TNR21, ZP3, SUSD4, DCBD1, HMR1, DISP1, IRPL1, ECSCR, or PLXC1 transmembrane domain comprising one or more antigen-inducible proteolytic cleavage sites, a juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

In one aspect, provided herein are primary immune cells comprising at least one recombinant nucleic acid comprising a priming receptor comprising an extracellular antigen-binding domain, a transmembrane domain comprising one or more antigen-inducible proteolytic cleavage sites, a PLXA2, NPTN, ALK, DSCAM, CD2, EPHA1, ERMAP, ERBB2, CEA20, KIRR1, C163A, PTPRF, CSPG4, SLAF1, or TMIG3 juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

In one aspect, provided herein are primary immune cells comprising at least one recombinant nucleic acid comprising a priming receptor comprising an extracellular antigen-binding domain, an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1 or NOTCH1 transmembrane domain comprising one or more antigen-inducible proteolytic cleavage sites, a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

In one aspect, provided herein are viable, virus-free, primary cells comprising a ribonucleoprotein complex (RNP)-recombinant nucleic acid complex, wherein the RNP comprises a nuclease domain and a guide RNA, wherein recombinant nucleic acid comprises a priming receptor comprises an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1 or NOTCH1 transmembrane domain comprising one or more antigen-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JDM), and a transcription factor; and a chimeric antigen receptor, and wherein the 5′ and 3′ ends of the recombinant nucleic acid comprise nucleotide sequences that are homologous to genomic sequences flanking an insertion site in the genome of the primary cell.

In one aspect, provided herein are populations of cells comprising a plurality of the primary immune cell disclosed herein.

In one aspect, provided herein are pharmaceutical compositions comprising the immune cell disclosed herein or the population of cells disclosed herein, and a pharmaceutically acceptable excipient.

In one aspect, provided herein are pharmaceutical compositions comprising the recombinant nucleic acid disclosed herein or the non-viral vector disclosed herein, and a pharmaceutically acceptable excipient.

In one aspect, provided herein are methods of editing an immune cell, comprising: providing a ribonucleoprotein complex (RNP)-recombinant nucleic acid complex, wherein the RNP comprises a nuclease domain and a guide RNA, wherein the recombinant nucleic acid comprises the recombinant nucleic acid disclosed herein, and wherein the 5′ and 3′ ends of the recombinant nucleic acid comprise nucleotide sequences that are homologous to genomic sequences flanking an insertion site in the genome of the immune cell; non-virally introducing the RNP-recombinant nucleic acid complex into the immune cell, wherein the guide RNA specifically hybridizes to a target region of the genome of the primary immune cell, and wherein the nuclease domain cleaves the target region to create the insertion site in the genome of the immune cell; and editing the immune cell via insertion of the recombinant nucleic acid disclosed herein into the insertion site in the genome of the immune cell.

In some embodiments, non-virally introducing comprises electroporation.

In some embodiments, the nuclease domain comprises a CRISPR-associated endonuclease (Cas), optionally a Cas9 nuclease.

In some embodiments, the target region of the genome of the cell is a T Cell Receptor Alpha Constant (TRAC) locus or a genomic safe harbor (GSH).

In some embodiments, the recombinant nucleic acid is a double-stranded recombinant nucleic acid or a single-stranded recombinant nucleic acid.

In some embodiments, the recombinant nucleic acid is a linear recombinant nucleic acid or a circular recombinant nucleic acid, optionally wherein the circular recombinant nucleic acid is a plasmid.

In some embodiments, the immune cell is a primary human immune cell.

In some embodiments, the primary immune cell is an autologous immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In some embodiments, the immune cell is virus-free.

In some embodiments, the method further comprises obtaining the immune cell from a patient and introducing the recombinant nucleic acid in vitro.

In one aspect, provided herein are methods of treating a disease in a subject comprising administering the immune cell disclosed herein or the pharmaceutical composition disclosed herein to the subject.

In some embodiments, the disease is cancer.

In some embodiments, the cancer is a solid cancer or a liquid cancer.

In some embodiments, the cancer is ovarian cancer, fallopian cancer, primary peritoneal cancer, uterine cancer, mesothelioma, cervical cancer, or pancreatic cancer.

In some embodiments, the method further comprises administering an immunotherapy to the subject concurrently with the immune cell or subsequently to the immune cell.

In one aspect, provided herein are methods of killing, disabling, depleting, or inhibiting a target cell in a subject comprising administering the immune cell disclosed herein to the subject, wherein the immune cell kills, disables, depletes, or inhibits the target cell.

In some embodiments, the target cell expresses ALPG/P and MSLN.

In some embodiments, the target cell is a cancer cell.

In one aspect, provided herein are methods of inducing expression of a chimeric antigen receptor with a priming receptor in an immune cell comprising: obtaining an immune cell comprising: the system disclosed herein; the recombinant nucleic acid disclosed herein; and/or the non-viral vector disclosed herein; and contacting the immune cell with a target cell expressing a first antigen to the priming receptor, wherein binding of the priming receptor to the first antigen on the target cell induces activation of the priming receptor and expression of the chimeric antigen receptor.

In one aspect, provided herein are methods of modulating the activity of an immune cell comprising: obtaining an immune cell comprising: the system disclosed herein; the recombinant nucleic acid disclosed herein; and/or the non-viral vector disclosed herein; and contacting the immune cell with a target cell expressing a first antigen and a second antigen, wherein binding of the priming receptor to the first antigen on the target cell induces activation of the priming receptor and expression of the chimeric antigen receptor and wherein binding of the chimeric antigen receptor to the second antigen on the target cell modulates the activity of the immune cell.

In some embodiments, the first antigen is an ALPG/P antigen.

In some embodiments, the second antigen is an MSLN antigen.

### BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 shows a diagram of an exemplary chimeric priming receptor.

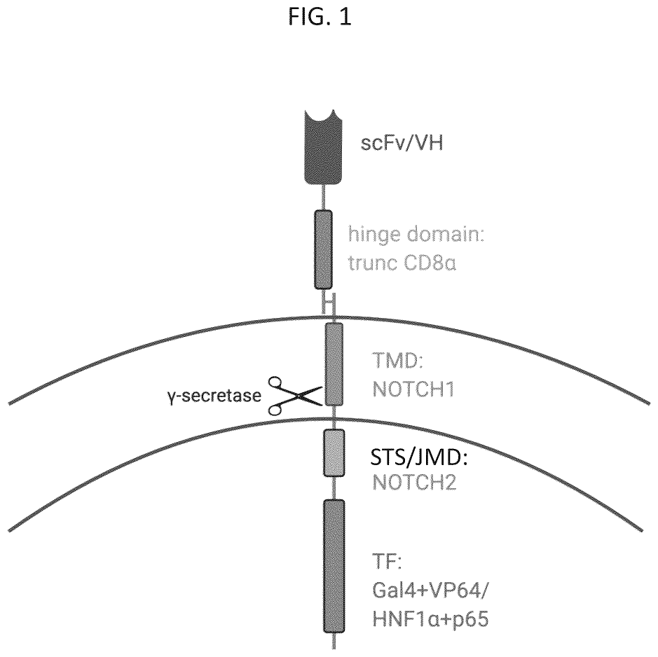

FIG. 2 provides preliminary screening hits for new transmembrane and intracellular juxtamembrane domains for use in priming receptors.

FIG. 3A shows CAR induction and basal priming receptor expression in T cells from donor 1 expressing the indicated priming receptor. FIG. 3B shows CAR induction and basal priming receptor expression in T cells from donor 2 expressing the indicated priming receptor.

FIG. 4 shows priming receptor induced cell cytotoxicity by T cells expressing the indicated priming receptor.

FIG. 5 shows priming receptor expression and CAR induction from additional screening hits.

FIG. 6 shows priming receptor to CAR conversion (turnover) from additional priming receptor screening hits.

FIG. 7 shows CAR induction and sensitive killing from additional priming receptor screening hits.

FIG. 8 shows CAR fidelity from additional priming receptor screening hits.

FIG. 9 shows results from a cytotoxicity assay by the indicated priming receptor T cells.

FIG. 10 shows CAR induction vs basal killing by the indicated priming receptor T cells.

FIG. 11 shows live cell killing by the indicated priming receptor T cells.

FIG. 12 shows live cell killing by the indicated priming receptor T cells expressing a large constitutive protein downstream of the prime receptor.

### DETAILED DESCRIPTION

### Definitions

Terms used in the claims and specification are defined as set forth below unless otherwise specified.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table E.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquizcae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-7, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+ double positive T cells, CD4+ helper T cells (e.g. Th1 and Th2 cells), CD8+ T cells (e.g. cytotoxic T cells), peripheral Including but not limited to blood mononuclear cells (PBMC), peripheral blood leukocytes (PBL), tumor infiltrating lymphocytes (TIL), memory T cells, naïve T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-7, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117+, lin⁻. In some cases, human hematopoietic stem cells are identified as CD34⁻, CD59⁺, Thy1/CD90⁺, CD38^{lo/-}, C-kit/CD117, lin⁻. In some cases, human hematopoietic stem cells are identified as CD133⁺, CD59⁺, Thy1/CD90⁺, CD38^{lo/-}, C-kit/CD117, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene recombinant DNA that has been cloned in a system that supports expression of the gene and translation of messenger RNA (see expression system). The recombinant protein can be a therapeutic agent, e.g. a protein that treats a disease or disorder disclosed herein. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). For example, an antibody that “selectively binds” or “specifically binds” an antigen is an antigen-binding moiety that binds the antigen with high affinity and does not significantly bind other unrelated antigens. Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including, but not limited to, surface plasmon resonance (SPR) technology (e.g., BIACORE®) or biolayer interferometry (e.g., FORTEBIO®).

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat, Chothia, AbM, Contact, and IMGT schemes. For CDR-H1, residue numbering is provided using both the Kabat and Chothia numbering schemes.

CDRs may be assigned, for example, using antibody numbering software, such as Abnum, available at bioinf.org.uk/abs/abnum/ and described in Abhinandan and Martin, Immunology, 2008, 45:3832-3839, incorporated by reference in its entirety, and AbYsis, available at abysis.org/abysis/sequence_input/key_annotation/key_annotation.cgi. Descriptions of the various antibody numbering schemes are available at bioinf.org.uk/abs/info.html.

TABLE A
Residues in CDRs according to according to the indicated numbering scheme.

| CDR | Kabat | Chothia | AbM | Contact | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 (Kabat Numbering) | H31-H35B | H26-H32 or H34* | H26-H35B | H30-H35B | H26-H35B |
| H1 (Chothia/Martin Numbering) | H31-H35 | H26-H32 | H26-H35 | H30-H35 | H26-H33 |
| H2 | H50-H65 | H52-H56 | H50-H58 | H47-H58 | H51-H56 |
| H3 | H95-H102 | H95-H102 | H95-H102 | H93-H101 | H93-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

As used herein, the term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region. “F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with 8-mercaptoethanol.

“Fv” fragments comprise a non-covalently-linked dimer of one heavy chain variable domain and one light chain variable domain.

The “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

It must be noted that, as used in the specification and the appended claims, the singular forms “a,” “an” and “the” include plural referents unless the context clearly dictates otherwise.

### Logic Gate Systems

As used herein, a “logic gate,” “circuit,” “circuit receptor,” “system” or “system receptor” refers to a two part protein expression system comprising a priming receptor and a chimeric antigen receptor. The system can be encoded on at least one nucleic acid inserted into a cell, where the priming receptor is expressed in the cell. The intracellular domain of the priming receptor is cleaved from the transmembrane domain upon binding of the priming receptor to its target antigen. The intracellular domain is then capable of translocating into a cell nucleus where it induces expression of the chimeric antigen receptor.

In one aspect, provided herein are systems comprising a priming receptor that binds to ALPG/P and a chimeric antigen receptor that binds to MSLN, wherein the transcription factor of the intracellular domain of the priming receptor is capable of inducing expression of the CAR. Such systems are alternatively termed “logic gates” or “circuits.” In some aspects, the system is encoded by nucleic acid transgenes inserted into an immune cell. The system can be encoded on a single nucleic acid insert or fragment that comprises both transgenes, or can be encoded on two nucleic acids that encode the system transgenes individually. The priming receptor and CAR of the system can be placed in any order on the single nucleic acid. For example, the priming receptor can be at the 5′ end and the CAR can be at the 3′ end, or the CAR can be at the 5′ end and the priming receptor can be at the 3′ end.

A constitutive promoter can be operably linked to the nucleotide sequence encoding the priming receptor. An inducible promoter can also be operably linked to the nucleotide sequence encoding the CAR. In some embodiments, when the system is encoded on a single nucleic acid insert or fragment that comprises both transgenes, the nucleic acid can comprise, in a 5′ to 3′ direction, the constitutive promoter; the nucleotide sequence encoding priming receptor; the inducible promoter; and the nucleotide sequence encoding chimeric antigen receptor. Alternatively, the nucleic acid can comprise, in a 5′ to 3′ direction, the inducible promoter; the nucleotide sequence encoding chimeric antigen receptor; the constitutive promoter; the nucleotide sequence encoding priming receptor.

### Priming Receptors

Previous syn-notch or hinge-notch receptors use Notch transmembrane and juxtamembrane domains. The use of these known domains limit the range of induction levels that are possible with the priming receptor. Additionally, the use of Notch1 or Notch2 transmembrane domain (TMD) and juxtamembrane domain (JMD) have a detectable level of basal activity. Provided herein are novel transmembrane and juxtamembrane domains that reduce the detectable level of basal activity. The new priming receptors disclosed herein replace the TMD and JMD with new domains that modulate the induction capacity of the receptor.

In some embodiments, the priming receptor comprises a sequence as set forth in SEQ ID NOs: 111-132.

In some embodiments, the priming receptor comprises a sequence as set forth in at least one of SEQ ID NO: 281-300.

Provided herein are priming receptors comprising in an N-terminus to C-terminus direction: an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites.

Also provided herein are priming receptors comprising, in an N-terminus to C-terminus direction: a transmembrane domain comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, or TMIG3 juxtamembrane domain (JMD); and an intracellular domain comprising a human or humanized transcriptional effector, wherein binding of the antigen to the extracellular antigen-binding domain results in cleavage at the one or more ligand-inducible proteolytic cleavage sites.

In some embodiments, the priming receptor further comprises an extracellular antigen-binding domain having a binding affinity for an antigen.

Also provided herein are priming receptors comprising, in an N-terminus to C-terminus direction: an extracellular antigen-binding domain having a binding affinity for Alkaline Phosphatase, Placental/Germ Cell (ALPG/P); an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, IRAG1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites.

In some embodiments, the priming receptor comprises, in an N-terminus to C-terminus direction: an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, IRAG1, HMR1, DISP1, IRPL1, ECSCR, or PLXC1 transmembrane domain (TMD) comprising one or more ligand-inducible proteolytic cleavage sites; and an intracellular domain comprising a human or humanized transcriptional effector, wherein binding of the antigen to the extracellular antigen-binding domain results in cleavage at the one or more ligand-inducible proteolytic cleavage sites. Exemplary priming receptor sequences comprising a novel TMD, JMD/STS, and intracellular effector domain are provided in SEQ ID NOs: 281-300. In some embodiments, the priming receptor comprises a sequence as set forth in SEQ ID NOs: 281-300.

Also provided herein are priming receptors comprising, in an N-terminus to C-terminus direction: an extracellular antigen-binding domain having a binding affinity for Alkaline Phosphatase, Placental/Germ Cell (ALPG/P); a transmembrane domain comprising one or more ligand-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, or TMIG3, juxtamembrane domain (JMD); and an intracellular domain comprising a human or humanized transcriptional effector, wherein binding of the antigen to the extracellular antigen-binding domain results in cleavage at the one or more ligand-inducible proteolytic cleavage sites.

In some embodiments, the priming receptor comprises an extracellular antigen-binding domain that specifically binds Alkaline Phosphatase, Placental (ALPP). In some embodiments, the priming receptor comprises an extracellular antigen-binding domain that specifically binds Alkaline Phosphatase, Germ Cell (ALPG). As used herein, “Alkaline Phosphatase, Placental/Germ Cell (ALPG/P)” refers to both Alkaline Phosphatase, Placental (ALPP) and Alkaline Phosphatase, Germ Cell (ALPG). An antigen binding domain that specifically binds ALPG/P is capable of specifically binding ALPG and/or ALPP.

In certain aspects of the present disclosure, the priming receptor is based on an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, IRAG1, HMR1, DISP1, IRPL1, ECSCR, PLXC1, or Notch protein. Binding of a natural receptor to a cognate ligand, such as those from the Delta family of proteins, causes intramembrane proteolysis that cleaves an intracellular fragment of the receptor. This intracellular fragment is a transcriptional regulator that only functions when cleaved from the receptor. Cleavage may occur by sequential proteolysis by ADAM metalloprotease and the gamma-secretase complex. This intracellular fragment enters the nucleus of a cell and activates cell-cell signaling genes. In contrast to a natural protein, a priming receptor replaces the natural intracellular fragment with one that causes a gene encoding a protein of choice, such as a CAR, to be transcribed upon release of the intracellular fragment from the priming receptor.

Table B provides the UniProt identifiers for the indicated human proteins from which the transmembrane and juxtamembrane domains are derived.

TABLE B

| Protein | UniProt Ref No. | Protein | UniProt Ref No. |
|---|---|---|---|
| STS | P08842 | PLXA2 | O75051 |
| SNORC | Q6UX34 | NPTN | Q9Y639 |
| SEM4F | O95754 | ALK | Q9UM73 |
| MCP | P15529 | DSCAM | Q0E9I4 |
| PLAT2 | Q9NWW9 | CD2 | P06729 |
| MMEL1 | Q495T6 | EPHA1 | P21709 |
| PLXC1 | O60486 | ERMAP | Q96PL5 |
| CLSTN1 | O94985 | ERBB2 | P04626 |
| CSTN2 | Q9H4D0 | CEA20 | Q6UY09 |
| SIGL9 | Q9Y336 | KIRR1 | Q96J84 |
| IRAG1 | Q9Y6F6 | C163A | Q86VB7 |
| TIGIT | Q495A1 | PTPRF | P10586 |
| TNR21 | O75509 | CSPG4 | Q6UVK1 |
| ZP3 | P21754 | SLAF1 | Q13291 |
| SUSD4 | Q5VX71 | TMIG3 | P0DMS9 |
| DCBD1 | Q8N8Z6 | NOTCH1 | P46531 |
| HMR1 | Q967F4 | NOTCH2 | Q04721 |
| DISP1 | Q95460 | | |
| IRPL1 | Q9NZN1 | | |
| ECSCR | Q19T08 | | |
| NOTCH1 | P46531 | | |

Priming receptor, such as Notch receptors, have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand or antigen binding domain, a linking polypeptide having substantial sequence identity with a Notch receptor including the NRR, a TMD, and an ICD. “Fn Notch” receptors comprise a heterologous extracellular ligand or antigen binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand or antigen binding domain, a linking polypeptide having substantial sequence identity with a Notch receptor (lacking the NRR), a TMD, and an ICD. “Minimal Linker Notch” receptors comprise a heterologous extracellular ligand or antigen binding domain, a linking polypeptide lacking substantial sequence identity with a Notch receptor (e.g., a synthetic (GGS)n polypeptide sequence), a TMD, and an ICD. “Hinge Notch” receptors comprise a heterologous extracellular ligand or antigen binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

### Priming Receptor Extracellular Domain

In some embodiments, the priming receptor extracellular domain includes the ligand or antigen-binding portion of a receptor. In some embodiments, the priming receptor extracellular domain includes an antigen-binding moiety that binds to one or more target antigens. In some embodiments, the priming receptor extracellular domain comprises means for binding one or more target antigens. In some embodiments, the means for binding the one or more target antigens comprises an antibody and antigen-binding fragment or equivalents thereof. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the priming receptor disclosed herein comprises an extracellular domain that specifically binds CD19; mesothelin (MSLN); Alkaline Phosphatase, Placental/Germ Cell (ALPG/P); epidermal growth factor receptor (EGFR); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In some embodiments, the priming receptor disclosed herein comprises an extracellular domain that specifically binds Alkaline Phosphatase, Placental/Germ Cell (ALPG/P). In some embodiments, the priming receptor extracellular antigen-binding domain specifically binds to Alkaline phosphatase, Germ Cell type (ALPG, UniProtKB—P10696). In some embodiments, the priming receptor extracellular domain includes an antigen-binding moiety that binds to Alkaline phosphatase, Placenta (ALPP, UniProtKB—P05187).

Various ALPG/P antibodies are known in the art, including but not limited to, the anti-ALPP antibodies disclosed in WO2017095823A1, the disclosure of which is hereby incorporated by reference in its entirety. Additional ALPP antibodies are available from commercial vendors such as ALPP/870 (cat #ab212383) from Abcam, OT11H2 (Catalog #TA506374) from Thermo Fisher, and 3E5C7 (cat #8681) from Cell Signaling Technology.

### Priming Receptor CDRs, VH, VL Domains

In some aspects, the priming receptor extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 88, 89, 90, 91, or 92; CDR-H2 comprises the sequence set forth in SEQ ID NO: 93, 94, 95, 96, 97; CDR-H3 comprises the sequence set forth in SEQ ID NO: 98, 99, 100; CDR-L1 comprises the sequence set forth in SEQ ID NO: 101, 102, 103; CDR-L2 comprises the sequence set forth in SEQ ID NO: 104, 105, or “NA”; and CDR-L3 comprises the sequence set forth in SEQ ID NO: 107 or 108. In some embodiments, the VH chain sequence comprises the sequence set forth in SEQ ID NO: 110. In some embodiments, the VL comprises the sequence set forth in SEQ ID NO: 109. In some embodiments, the extracellular domain comprises the sequence set forth in SEQ ID NO: 76.

In some embodiments, the priming receptor extracellular antigen-binding domain CDR-H3 has at least about 50%, 75%, 80%, 85%, 90%, or 95% identity with a CDR-H3 of SEQ ID NO: 98, 99, 100, the CDR-H2 has at least about 50%, 75%, 80%, 85%, 90%, or 95% identity with a CDR-H2 of SEQ ID NO: 93, 94, 95, 96, 97, the CDR-H1 has at least about 50%, 75%, 80%, 85%, 90%, or 95% identity with a CDR-H1 of SEQ ID NO: 88, 89, 90, 91, or 92, the CDR-L3 has at least about 50%, 75%, 80%, 85%, 90%, or 95% identity with a CDR-L3 of SEQ ID NO: 107 or 108, the CDR-L2 has at least about 50%, 75%, 80%, 85%, 90%, or 95% identity with a CDR-L2 of SEQ ID NO: 104, 105, or “NA”, and the CDR-L1 has at least about 50%, 75%, 80%, 85%, 90%, or 95% identity with a CDR-L1 of SEQ ID NO: 101, 102, 103. In some embodiments, the CDR-H3 is a CDR-H3 of SEQ ID NO: 98, 99, 100, with up to 1, 2, 3, 4, 5, 6, 7, or 8 amino acid substitutions; the CDR-H2 is a CDR-H2 of SEQ ID NO: 93, 94, 95, 96, 97, with up to 1, 2, 3, 4, 5, 6, 7, or 8 amino acid substitutions; the CDR-H1 is a CDR-H1 of SEQ ID NO: 88, 89, 90, 91, or 92, with up to 1, 2, 3, 4, or 5 amino acid substitutions; the CDR-L3 is a CDR-L3 of SEQ ID NO: 107 or 108, with up to 1, 2, 3, 4, or 5 amino acid substitutions; the CDR-L2 is a CDR-L2 of SEQ ID NO: 104, 105, or “NA”, with up to 1, 2, 3, or 4 amino acid substitutions; and the CDR-L1 is a CDR-L1 of SEQ ID NO: 101, 102, 103 with up to 1, 2, 3, 4, 5, or 6 amino acid substitutions.

In some embodiments, a priming receptor extracellular antigen-binding domain provided herein comprises one to three CDRs of a VH domain as set forth in SEQ ID NO: 110. In some embodiments, an antigen-binding domain provided herein comprises two to three CDRs of a VH domain as set forth in SEQ ID Ns: 110. In some embodiments, an antigen-binding domain provided herein comprises three CDRs of a VH domain as set forth in SEQ ID NO: 110. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a priming receptor extracellular antigen-binding domain provided herein comprises a VH sequence having at least about 50%, 60%, 70%, 80%, 90%, 95%, or 99% identity to an VH sequence set forth in SEQ ID NO: 110. In some embodiments, an antigen-binding domain provided herein comprises a VH sequence provided in SEQ ID NO: 110, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a priming receptor extracellular antigen-binding domain provided herein comprises one to three CDRs of a VL domain as set forth in SEQ ID NO: 109. In some embodiments, an antigen-binding domain provided herein comprises two to three CDRs of a VL domain as set forth in SEQ ID NO: 109. In some embodiments, an antigen-binding domain provided herein comprises three CDRs of a VL domain as set forth in SEQ ID NO: 109. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a priming receptor extracellular antigen-binding domain provided herein comprises a VL sequence having at least about 50%, 60%, 70%, 80%, 90%, 95%, or 99% identity to an VL sequence set forth in SEQ ID NO: 109. In some embodiments, a antigen-binding domain provided herein comprises a VL sequence provided in SEQ ID NO: 109, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

TABLE C
Table C provides the CDR sequences of the VH and
VL of an illustrative ALPG/P antigen binding
domain according to the indicated
numbering schemes.

| SEQ | | | |
|---|---|---|---|
| ID | | Numbering | |
| NO | Name | scheme | Sequence |
| 88 | CDR-H1 | Chothia | GFSLTSY--- |
| 89 | | AbM | GFSLTSYGVS |
| 90 | | Kabat | -----SYGVS |
| 91 | | Contact | ----TSYGVS |
| 92 | | IMGT | GFSLTSYG-- |
| 93 | CDR-H2 | Chothia | -----WEDGS--------- |
| 94 | | AbM | ---VIWEDGSTN------- |
| 95 | | Kabat | ---VIWEDGSTNYHSALIS |
| 96 | | Contact | WIGVIWEDGSTN------- |
| 97 | | IMGT | ----IWEDGST-------- |
| 98 | CDR-H3 | Chothia | --PHYGSSYVGAMEY |
| 98 | | AbM | --PHYGSSYVGAMEY |
| 98 | | Kabat | --PHYGSSYVGAMEY |
| 99 | | Contact | ARPHYGSSYVGAME- |
| 100 | | IMGT | ARPHYGSSYVGAMEY |
| 101 | CDR-L1 | Chothia | RASENIYSYVA-- |
| 101 | | AbM | RASENIYSYVA-- |
| 101 | | Kabat | RASENIYSYVA-- |
| 102 | | Contact | ------YSYVAWY |
| 103 | | IMGT | ---ENIYSY---- |
| 104 | CDR-L2 | Chothia | ----NAKSLAS |
| 104 | | AbM | ----NAKSLAS |
| 104 | | Kabat | ----NAKSLAS |
| 105 | | Contact | LLIYNAKSLA- |
| — | | IMGT | ----NA----- |
| 107 | CDR-L3 | Chothia | QHHYVSPWT |
| 107 | | AbM | QHHYVSPWT |
| 107 | | Kabat | QHHYVSPWT |
| 108 | | Contact | QHHYVSPW- |
| 107 | | IMGT | QHHYVSPWT |

### Transmembrane Domain

As described above, the priming receptor comprises a transmembrane domain (TMD) comprising one or more ligand-inducible proteolytic cleavage sites.

In some embodiments, the transmembrane domain comprises a STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1, or NOTCH1 transmembrane domain. In some embodiments, the transmembrane domain comprises the sequence as set forth in SEQ ID NO: 1-21.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014).

In some embodiments, the priming receptor comprises a Notch cleavage site, such as S2 or S3. Additional proteolytic cleavage sites suitable for the compositions and methods disclosed herein include, but are not limited to, ADAM10, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 155), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 156), where cleavage occurs after the lysine residue. Another example of a protease cleavage site that can be included in a proteolytically cleavable linker is a thrombin cleavage site, e.g., Leu-Val-Pro-Arg (SEQ ID NO: 157). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator(tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

### Juxtamembrane Domain and Stop-Transfer Sequence

The extracellular domain and the transmembrane domain, or the transmembrane domain and the intracellular domain, can be linked to each other with a linking polypeptide, such as a juxtamembrane domain or a stop transfer sequence (STS). Juxtamembrane domains can therefore be present in the extracellular portion of the priming receptor or the intracellular portion of the priming receptor.

In some embodiments, the priming receptor further comprises a juxtamembrane domain (JMD) in between the transmembrane domain and the intracellular domains. Intracellular JMDs can also be referred to as a stop-transfer sequence (STS). The JMD/STS can comprise a charged, lipophobic sequence. Without being bound by any theory, the JMD/STS serves as a membrane anchor, and is believed to prevent passage of the intracellular domain into the plasma membrane. The use of juxtamembrane domains or stop-transfer sequences in priming receptors is described in WO2021061872, hereby incorporated by reference in its entirety. In some embodiments, the juxtamembrane domain or stop-transfer sequence is heterologous to the transmembrane domain. In some embodiments, the juxtamembrane domain or stop-transfer sequence is homologous to the transmembrane domain.

In some embodiments, the juxtamembrane domain is selected from the group consisting of PLXA2, NPTN, ALK, DSCAM, CD2, EPHA1, ERMAP, ERBB2, CEA20, KIRR1, C163A, PTPRF, CSPG4, SLAF1, TMIG3, NOTCH1, and NOTCH2 juxtamembrane domains. In some embodiments, the juxtamembrane domain comprises a sequence selected from the sequences set forth in SEQ ID NOs: 22-39.

In some embodiments, the transmembrane domain and juxtamembrane domain are selected from the combinations consisting of: NOTCH1-PLXA2, CLSTN1-CD2, NOTCH1-NPTN, NOTCH1-ALK, SNORC-NOTCH1, SEM4F-NOTCH2, STS-NOTCH2, MCP-NOTCH1, DCBD1-NOTCH2, IRAG1-C163A, HMR1-NOTCH1, MCP-NOTCH1, DISP1-NOTCH2, IRPL1-NOTCH1, ECSCR-NOTCH1, NOTCH1-PTPRF, NOTCH1-CSPG4, SEM4F-NOTCH2, IRAG1-CD2, SIGL9-CD2, IRAG1-NOTCH1, NOTCH1-SLAF1, NOTCH1-TMIG3, NOTCH1-C163A, and PLXC1-EPHA1.

In some embodiments, the transmembrane domain and juxtamembrane domain comprise a sequence selected from the sequences set forth in SEQ ID NOs: 40-57. In some embodiments, the transmembrane domain and juxtamembrane domain is encoded from a nucleic acid sequence selected from the sequences set forth in SEQ ID NOs: 58-75.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligimerization domain which promotes formation of dimers, trimers, or higher order assemblages of the receptor. Such JMD peptides are described in WO2021061872, hereby incorporated by reference in its entirety.

### Hinge

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO: 158).

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8 α hinge domain or a functional variant thereof. In some embodiments, the hinge domain includes a hinge polypeptide sequence derived from a CD8α hinge domain or a functional variant thereof. In some embodiments, the hinge domain includes a hinge polypeptide sequence derived from a truncated CD8α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

In some embodiments, the priming receptor hinge comprises the sequence set forth in SEQ ID NO: 83.

### Intracellular Domain

In some embodiments, the priming receptor comprises one or more intracellular domains from or derived from a transcriptional regulator and/or a DNA-binding domain. In some embodiments, the intracellular domain comprises an HNF1a/p65 domain or a Gal4/VP64 domain. In some embodiments, the intracellular domain comprises the sequence as set forth in SEQ ID NO: 80, 81, or 82.

Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (cAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

In some embodiments, the priming receptor comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1a, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu196, and TALES.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

### Chimeric Antigen Receptors

In another aspect, provided herein are systems comprising a priming receptor and a chimeric antigen receptor comprising an extracellular antigen-binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some aspects, the chimeric antigen receptor includes an extracellular portion comprising an antigen binding domain described herein and an intracellular signaling domain. In some embodiments, an antibody or fragment includes an scFv, a VH, or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In another aspect, provided herein are chimeric antigen receptors comprising an extracellular antigen-binding domain that specifically binds to mesothelin (MSLN). The recombinant CAR may be a human CAR, comprising fully human sequences, e.g., natural human sequences.

In some aspects, the transmembrane domain contains a transmembrane portion of CD8α or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

### CAR Extracellular Domain

In some embodiments, the CAR extracellular domain includes the ligand or antigen-binding portion of a receptor. In some embodiments, the CAR extracellular domain includes an antigen-binding moiety that binds to one or more target antigens. In some embodiments, the CAR extracellular domain comprises means for binding one or more target antigens. In some embodiments, the means for binding the one or more target antigens comprises an antibody and antigen-binding fragment or equivalents thereof. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, the CAR disclosed herein comprises an extracellular domain that specifically binds CD19; mesothelin (MSLN); Alkaline Phosphatase, Placental/Germ Cell (ALPG/P); epidermal growth factor receptor (EGFR); CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In some embodiments, the CAR disclosed herein comprises an extracellular domain that specifically binds mesothelin (MSLN, UniProtKB—H3BR90). Various mesothelin antibodies or antigen fragments are known in the art, including, but not limited to, the anti-mesothelin (MSLN) antibodies and antigen binding fragments disclosed in WO2006099141A2, US20170267755A1, U.S. Pat. Nos. 8,206,710, 9,023,351, 9,416,190, and 7,943,133, the disclosure of each of which is hereby incorporated by reference in its entirety.

### Chimeric Antigen Receptor CDRs, VH, VL Domains

In some aspects, the priming receptor extracellular antigen-binding domain comprises a variable heavy (VH) chain sequence comprising three heavy chain CDR sequences, CDR-H1, CDR-H2, and CDR-H3, and a variable light (VL) chain sequence comprising three light chain CDR sequences, CDR-L1, CDR-L2, and CDR-L3, wherein: CDR-H1 comprises the sequence set forth in SEQ ID NO: 133, 141, 142, 143, or 144, CDR-H2 comprises the sequence set forth in SEQ ID NO: 134, 145, 146, 147, or 148, CDR-H3 comprises the sequence set forth in SEQ ID NO: 135, 149, or 150, CDR-L1 comprises the sequence set forth in SEQ ID NO: 137, 151, or 152, CDR-L2 comprises the sequence set forth in SEQ ID NO: 138 or 153; and CDR-L3 comprises the sequence set forth in SEQ ID NO: 139 or 154. In some embodiments, the VH chain sequence comprises the sequence set forth in SEQ ID NO: 136. In some embodiments, the VL comprises the sequence set forth in SEQ ID NO: 140. In some embodiments, the antigen-binding domain comprises the sequence set forth in SEQ ID NO: 78.

In some embodiments, the CAR extracellular antigen-binding domain CDR-H3 has at least about 50%, 75%, 80%, 85%, 90%, or 95% identity with a CDR-H3 of SEQ ID NO: 135, 149, or 150, the CDR-H2 has at least about 50%, 75%, 80%, 85%, 90%, or 95% identity with a CDR-H2 of SEQ ID NO: 134, 145, 146, 147, or 148, the CDR-H1 has at least about 50%, 75%, 80%, 85%, 90%, or 95% identity with a CDR-H1 of SEQ ID NO: 133, 141, 142, 143, or 144, the CDR-L3 has at least about 50%, 75%, 80%, 85%, 90%, or 95% identity with a CDR-L3 of SEQ ID NO: 139 or 154, the CDR-L2 has at least about 50%, 75%, 80%, 85%, 90%, or 95% identity with a CDR-L2 of SEQ ID NO: 138 or 153, and the CDR-L1 has at least about 50%, 75%, 80%, 85%, 90%, or 95% identity with a CDR-L1 of SEQ ID NO: 137, 151, or 152. In some embodiments, the CDR-H3 is a CDR-H3 of SEQ ID NO: 135, 149, or 150, with up to 1, 2, 3, 4, 5, 6, 7, or 8 amino acid substitutions; the CDR-H2 is a CDR-H2 of SEQ ID NO: 134, 145, 146, 147, or 148, with up to 1, 2, 3, 4, 5, 6, 7, or 8 amino acid substitutions; the CDR-H1 is a CDR-H1 of SEQ ID NO: 133, 141, 142, 143, or 144, with up to 1, 2, 3, 4, or 5 amino acid substitutions; the CDR-L3 is a CDR-L3 of SEQ ID NO: 139 or 154, with up to 1, 2, 3, 4, or 5 amino acid substitutions; the CDR-L2 is a CDR-L2 of SEQ ID NO: 138 or 153, with up to 1, 2, 3, or 4 amino acid substitutions; and the CDR-L1 is a CDR-L1 of SEQ ID NO: 137, 151, or 152 with up to 1, 2, 3, 4, 5, or 6 amino acid substitutions.

In some embodiments, a CAR extracellular antigen-binding domain provided herein comprises one to three CDRs of a VH domain as set forth in SEQ ID NO: 136. In some embodiments, an antigen-binding domain provided herein comprises two to three CDRs of a VH domain as set forth in SEQ ID NO: 136. In some embodiments, an antigen-binding domain provided herein comprises three CDRs of a VH domain as set forth in SEQ ID NO: 136. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a CAR extracellular antigen-binding domain provided herein comprises a VH sequence having at least about 50%, 60%, 70%, 80%, 90%, 95%, or 99% identity to an VH sequence set forth in SEQ ID NO: 136. In some embodiments, an antigen-binding domain provided herein comprises a VH sequence provided in SEQ ID NO: 136, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antigen-binding domains described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

In some embodiments, a CAR extracellular antigen-binding domain provided herein comprises one to three CDRs of a VL domain as set forth in SEQ ID NO: 140. In some embodiments, an antigen-binding domain provided herein comprises two to three CDRs of a VL domain as set forth in SEQ ID NO: 140. In some embodiments, an antigen-binding domain provided herein comprises three CDRs of a VL domain as set forth in SEQ ID NO: 140. In some aspects, the CDRs are Kabat CDRs. In some aspects, the Kabat VH CDRs are provided in the sequences set forth as SEQ ID NOs: 133, 134, and 135, and the Kabat VL CDRs are provided in the sequences set forth as SEQ ID NOs: 137, 138, and 139. In some aspects, the CDRs are Chothia CDRs. In some aspects, the Chothia VH CDRs are provided in the sequences set forth as SEQ ID NOs: 141, 145, and 135, and the Chothia VL CDRs are provided in the sequences set forth as SEQ ID NOs: 137, 138, and 139. In some aspects, the CDRs are AbM CDRs. In some aspects, the AbM VH CDRs are provided in the sequences set forth as SEQ ID NOs: 142, 146, and 135, and the AbM VL CDRs are provided in the sequences set forth as SEQ ID NOs: 137, 138, and 139. In some aspects, the CDRs are Contact CDRs. In some aspects, the Contact VH CDRs are provided in the sequences set forth as SEQ ID NOs: 143, 147, and 149, and the Contact VL CDRs are provided in the sequences set forth as SEQ ID NOs: 151, 153, and 154. In some aspects, the CDRs are IMGT CDRs. In some aspects, the IMGT VH CDRs are provided in the sequences set forth as SEQ ID NOs: 144, 147, and 149, and the IMGT VL CDRs are provided in the sequences set forth as SEQ ID NOs: 152 and 139 and the sequence DT.

In some embodiments, a CAR extracellular antigen-binding domain provided herein comprises a VL sequence having at least about 50%, 60%, 70%, 80%, 90%, 95%, or 99% identity to an VL sequence set forth in SEQ ID NO: 140. In some embodiments, an antigen-binding domain provided herein comprises a VL sequence provided in SEQ ID NO: 140, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies or antigen-binding domains.

TABLE D
Table D provides illustrative MSLN antigen
binding domain CDR sequences of the VH of
SEQ ID NO: 136 and the VL of SEQ ID NO: 140,
according to the indicated numbering schemes.

| SEQ | | | |
|---|---|---|---|
| ID | | Numbering | |
| NO | Name | scheme | Sequence |
| 141 | CDR-HI | Chothia | GYSFTGY |
| 142 | | AbM | GYSFTGYTMN |
| 133 | | Kabat | GYTMN |
| 143 | | Contact | TGYTMN |
| 144 | | IMGT | GYSFTGYT |
| 145 | CDR-H2 | Chothia | TPYNGA |
| 146 | | AbM | LITPYNGASS |
| 134 | | Kabat | LITPYNGASSYNQKFRG |
| 147 | | Contact | WIGLITPYNGASS |
| 148 | | IMGT | ITPYNGAS |
| 135 | CDR-H3 | Chothia | GGYDGRGFDY |
| 135 | | AbM | GGYDGRGFDY |
| 135 | | Kabat | GGYDGRGFDY |
| 149 | | Contact | ARGGYDGRGFD |
| 150 | | IMGT | ARGGYDGRGFDY |
| 137 | CDR-L1 | Chothia | SASSSVSYMH |
| 137 | | AbM | SASSSVSYMH |
| 137 | | Kabat | SASSSVSYMH |
| 151 | | Contact | SYMHWY |
| 152 | | IMGT | SSVSY |
| 138 | CDR-L2 | Chothia | DTSKLAS |
| 138 | | AbM | DTSKLAS |
| 138 | | Kabat | DTSKLAS |
| 153 | | Contact | RWIYDTSKLA |
| — | | IMGT | DT |
| 139 | CDR-L3 | Chothia | QQWSGYPLT |
| 139 | | AbM | QQWSGYPLT |
| 139 | | Kabat | QQWSGYPLT |
| 154 | | Contact | QQWSGYPL- |
| 139 | | IMGT | QQWSGYPLT |

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the CAR comprises a CD8α TMD. In some embodiments, the CD8α TMD comprises the sequence set forth in SEQ ID NO: 85.

### CAR Hinge

In some embodiments, the CAR further includes a spacer, which may be or include at least a portion of an immunoglobulin constant region or variant or modified version thereof, such as a hinge region, e.g., a CD8α hinge, an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8α hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8α hinge. In some embodiments, the CD8α hinge comprises the sequence set forth in SEQ ID NO: 84.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16. In some embodiments, the CAR comprises a CD3-zeta activation domain comprising the sequence set forth in SEQ ID NO: 87.

In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD8α transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain. In some embodiments, the 4-1BB co-stimulatory domain comprises the sequence as set forth in SEQ ID NO: 86.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g. containing only the VH region), VH domains, and scFvs, described herein, a spacer such as a CD8α hinge, a CD8α transmembrane domain, a 4-1BB intracellular signaling domain, and a CD3 zeta signaling domain. In some embodiments, the CAR includes an antibody or fragment, including sdAbs and scFvs described herein, a spacer such as a CD8α hinge, a CD8α transmembrane domain, a 4-1BB intracellular signaling domain, and a CD3 zeta signaling domain.

Transgenes expressing the priming receptor and CAR system may be introduced into cells, such as a T cell, using, for example, a site-specific technique. With site specific integration of the transgenes (e.g. priming receptor and CAR), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered cells have applications to immune-oncology. The priming receptor and CAR, for example, can be selected to target different specific tumor antigens. Examples of cancers that can be effectively targeted using such cells are blood cancers or solid cancers. In some embodiments, immune cell therapy can be used to treat solid tumors.

In some embodiments, the CAR comprises the sequence set forth in SEQ ID NO: 79.

### Recombinant Nucleic Acids and Vectors

In some embodiments, the present disclosure contemplates recombinant nucleic acid inserts that comprise one or more transgenes encoding the priming receptors and/or CARs as described herein. In some embodiments, the insert encodes a priming receptor transgene. In some embodiments, the insert encodes a chimeric antigen receptor transgene. In some embodiments, the insert comprises a priming receptor transgene and a chimeric antigen receptor transgene.

The insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7(2): 403-412 (2015)).

The insert can also comprise a WPRE element. WPRE elements are generally described in Higashimoto, T., et al. Gene Ther 14, 1298-1304 (2007); and Zufferey, R., et al. J Virol. 1999 April; 73(4):2886-92., both of which are hereby incorporated by reference.

### Recombinant Cells

Also provided herein are recombinant immune cells comprising at least one DNA template non-virally inserted into a target region of the genome of the cell, wherein DNA template encodes the priming receptor and CAR system as described herein. Also provided herein are recombinant immune cells comprising the priming receptor that specifically binds Alkaline Phosphatase, Placental/Germ Cell (ALPG/P) and the chimeric antigen receptor that specifically binds MSLN.

A cell comprising a DNA template insert at a target locus or safe harbor site as described in the present disclosure can be referred to as an engineered cell. In some embodiments, the immune cell is any cell that can give rise to a pluripotent immune cell. In some embodiments, the immune cell is a primary immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an hematopoietic stem cell, an adaptive immune cell, an innate immune cell, a T cell or a T cell progenitor. Non-limiting examples of immune cells that are contemplated in the present disclosure include T cell, B cell, natural killer (NK) cell, NKT/iNKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

Also provided herein are populations of cells comprising a plurality of the immune cell. In some embodiments, the genome of at least 20%, 30%, 40%, 50%, 60%, 70%, 80%, 90%, 95%, 99% or greater of the cells comprises the priming receptor and CAR system as described herein.

### Method of Treating Cancer

In another aspect, provided herein are methods of treating an immune-related condition (e.g., cancer) in an individual comprising administering to the individual an effective amount of a composition comprising a system comprising a priming receptor comprising a TMD and JMD as disclosed herein and a chimeric antigen receptor. In another aspect, provided herein are methods of enhancing an immune response in an individual comprising administering to the individual an effective amount of a composition comprising a system comprising a TMD and JMD as disclosed herein and a chimeric antigen receptor. In another aspect, provided herein are methods of inducing cytolytic activity of an immune cell (e.g., a T cell) in an individual comprising administering to the individual an effective amount of a composition comprising a system comprising a TMD and JMD as disclosed herein and a chimeric antigen receptor. In some embodiments, the priming receptor specifically binds to ALPG/P and the chimeric antigen receptor specifically binds to MSLN.

In some embodiments, the methods provided herein are useful for the treatment of an immune-related condition in an individual. In one embodiment, the individual is a human.

In some embodiments, the methods provided herein (such as methods of enhancing an immune response) are useful for the treatment of cancer and as such an individual receiving the system described herein has cancer. In some embodiments, the cancer is a solid cancer. In some embodiments, the cancer is a liquid cancer. In some embodiments, the cancer is immunoevasive. In some embodiments, the cancer is immunoresponsive. In particular embodiments, the cancer is ovarian cancer, fallopian cancer, primary peritoneal cancer, uterine cancer, mesothelioma, cervical cancer, or pancreatic cancers. In particular embodiments, the cancer is ovarian cancer.

In some embodiments, the treatment results in a decrease in the cancer volume or size. In some embodiments, the treatment is effective at reducing a cancer volume as compared to the cancer volume prior to administration of the antibody. In some embodiments, the treatment results in a decrease in the cancer growth rate. In some embodiments, the treatment is effective at reducing a cancer growth rate as compared to the cancer growth rate prior to administration of the antibody. In some embodiments, the treatment is effective at eliminating the cancer.

In some embodiments, the priming receptor antigenic target (e.g., the antigen to which the priming receptor extracellular domain binds) and the CAR antigenic target (e.g., the antigen to which the CAR extracellular domain binds) are expressed at a higher level in the cancer as compared to a non-cancer cell. In some embodiments, MSLN and ALPG or ALPP are expressed at a higher level in the cancer as compared to a non-cancer cell. Levels of the priming receptor and CAR antigenic targets, e.g., MSLN and ALPG/ALPP, can be assessed by any technique known in the field, including, but not limited to, protein assays or nucleic assays such as FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, qPCR, RT-qPCR, multiplex qPCR or RT-qPCR, RNA-seq, microarray analysis, SAGE, MassARRAY technique, and FISH, and combinations thereof.

### Method of Immune Modulation

Methods of administration of a cell comprising a system comprising a priming receptor comprising a TMD and JMD as disclosed herein and a chimeric antigen receptor as described herein can result in modulation of an immune response. In some embodiments, the system comprises a priming receptor that specifically binds to ALPG/P and a chimeric antigen receptor that specifically binds to MSLN. Modulation can be an increase or decrease in an immune response. In some embodiments, modulation is an increase in an immune response.

In one aspect, administration of a cell comprising a system comprising a priming receptor comprising a TMD and JMD as disclosed herein and a chimeric antigen receptor can result in induction of pro-inflammatory molecules, such as cytokines or chemokines. In some embodiments, the system comprises a priming receptor that specifically binds to ALPG/P and a chimeric antigen receptor that specifically binds to MSLN. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a system comprising a comprising a TMD and JMD as disclosed herein and a chimeric antigen receptor can induce multiple anti-tumor immune mechanisms that lead to tumor destruction.

In another aspect, provided herein are methods of increasing an immune response in an individual comprising administering to the individual an effective amount of a cell comprising a TMD and JMD as disclosed herein and a chimeric antigen receptor. In some embodiments, the method of increasing an immune response in a subject comprises administering to the subject a cell comprising a system comprising a TMD and JMD as disclosed herein and a chimeric antigen receptor. In some embodiments, the priming receptor specifically binds to ALPG/P and the chimeric antigen receptor specifically binds to MSLN.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a composition comprising a system comprising a TMD and JMD as disclosed herein and a chimeric antigen receptor.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a system comprising a priming receptor that specifically binds to ALPG/P and a chimeric antigen receptor that specifically binds to MSLN. In some embodiments, the immune response is enhanced by administration of cell comprising a system comprising a priming receptor that specifically binds to ALPG/P and a chimeric antigen receptor that specifically binds to MSLN.

In another aspect, the present application provides methods of genetically editing a cell with a system comprising a priming receptor that specifically binds to ALPG/P and a chimeric antigen receptor that specifically binds to MSLN, which results in the modulation of the immune function of the cell. The modulation can be increasing an immune response. In some embodiments, the modulation is an increase in immune function. In some embodiments, the modulation of function leads to the expression of an MSLN CAR. In some embodiments, the modulation of function leads to the activation of a cell comprising the system.

In some embodiments, the cell is a natural killer (NK) cell, a T cell, a CD8+ T cell, a CD4+ T cell, a primary T cell, or a T cell progenitor.

In some embodiments, the modulation of function of the cells comprising the priming receptor and CAR system as described herein leads to an increase in the cells' abilities to stimulate both native and activated T-cells, for example, by increasing cytokine or chemokine secretion by the cells expressing the priming receptor and CAR system. In some embodiments, the modulation of function enhances or increases the cells' ability to produce cytokines, chemokines, CARs, or costimulatory or activating receptors. In some embodiments, the modulation increases the T-cell stimulatory function of the cells expressing the priming receptor and CAR system, including, for example, the cells' abilities to trigger T-cell receptor (TCR) signaling, T-cell proliferation, or T-cell cytokine production.

In some embodiments, the increased immune response is secretion of cytokines and chemokines. In some embodiments, the priming receptor and CAR system induces increased expression of at least one cytokine or chemokine in a cell as compared to an isotype control cell. In some embodiments, the at least one cytokine or chemokine is selected from the group consisting of: IL-2 and IFNg. In some embodiments, the cytokine or chemokine is IL-2. In some embodiments, the cytokine or chemokine is IFNg. In some embodiments, the cytokine or chemokine secretion is increased a between bout 1-100-fold 1, 5, 10, 20, 30, 40, 50, 60, 70, 80, 90, 100, 1-10, 10-20, 20-30, 30-40, 40-50, 50-60, 60-70, 70-80, 80-90, or 90-100 fold as compared to an untreated cell or a cell treated with an isotype control antibody. In some embodiments, the chemokine is IL-2 and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the cytokine is IFNg and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody.

In some embodiments, the enhanced immune response is anti-tumor immune cell recruitment and activation.

In some embodiments, the cell expressing the priming receptor and CAR system induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

The plasmid can be introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit system receptors do not express CAR until the priming receptor binds to its cognate ligand and releases the cleavable transcription factor.

Initially, a T cell is activated. The T cell may be obtained from a patient. Thus, the present disclosure provides methods in which immune cells, such as T cells, are harvested from a patient. Then, the plasmid that encodes the CAR and priming receptor are introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The T cells are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the size of the DNA template is greater than about 200 nucleotides, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the DNA template is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10 kb or any size of DNA template in between these sizes. For example, the size of the DNA template can be about 4.5 kb to about 10 kb, about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the DNA template is large enough and in sufficient quantity to be lethal as naked DNA. In some embodiments, the DNA template encodes a heterologous protein or a fragment thereof. In some embodiments, the DNA template encodes at least one gene. In some embodiments, the DNA template encodes at least two genes. In some embodiments, the DNA template encodes one, two, three, four, five, six, seven, eight, nine, ten, or more genes.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

### Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of a T cell, specifically, include a method of editing the genome of a human T cell comprise inserting a nucleic acid sequence or construct into a target region of a genomic safe harbor (GSH).

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12(1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. An exemplary AAVS1 target gRNA and target sequence are provided below:

AAVS1-gRNA sequence (SEQ ID NO: 159):
ggggccactagggacaggatGTTTTAGAGCTAGAAAT
AGCAAGTTAAAATAAGGCTAGTCCGTTATCAACTTGA
AAAAGTGGCACCGAGTCGGTGCTTTTTTT
AAVS1 target sequence (SEQ ID NO: 160):
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. Examples of additional integration sites are provided in Table E.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 kB, about 525 kB, about 550 kB away from the safe harbor locus (integration site).

In some embodiments, the present disclosure contemplates inserts that comprise one or more transgenes. The transgene can encode a therapeutic protein, an antibody, a peptide, or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, a T cell having “enhanced therapeutic properties” has an enhanced, improved, and/or increased treatment outcome when compared to a typical, unmodified and/or naturally occurring T cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kilobasepairs (kb) to about 10 kilobasepairs (kb). In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kbp (kilo basepairs) or the sizes in between. In some embodiments, the insert size is greater than 4.5, 4.8, 5, 6, 7, 8, 9, 10, 11, 12, 13, 14, 15, 16, 17, 18, 19, 20 kbp or the sizes in between. In some embodiments, the insert size is within the range of 4.5-15 kbp or is any number in that range. In some embodiments, the insert size is within the range of 4.8-8.3 kbp or is any number in that range. In some embodiments, the insert size is within the range of 5-8.3 kbp or is any number in that range. In some embodiments, the insert size is within the range of 5-15 kbp or is any number in that range. In some embodiments, the insert size is within the range of 4.5-20 kbp or is any number in that range. In some embodiments, the insert size is 5-10 kbp. In some embodiments, the insert size is 4.5-10, 5-10, 6-10, 7-10, 8-10, 9-10 kbp. In some embodiments, the insert size is 4.5-11, 6-11, 7-11, 8-11, 9-11, or 10-11 kbp. In some embodiments, the insert size is 4.5-12, 6-12, 7-12, 8-12, 9-12, 10-12, or 11-12 kbp. In some embodiments, the insert size is 4.5-13, 6-13, 7-13, 8-13, 9-13, 10-13, 11-13, or 12-13 kbp. In some embodiments, the insert size is 4.5-14, 6-14, 7-14, 8-14, 9-14, 10-14, 11-14, 12-14 or 13-14 kbp. In some embodiments, the insert size is 4.5-15, 6-15, 7-15, 8-15, 9-15, 10-15, 11-15, 12-15, 13-15, or 14-15 kbp. In some embodiments, the insert size is 4.5-16, 6-16, 7-16, 8-16, 9-16, 10-16, 11-16, 12-16, 13-16, 14-16 or 15-16 kbp. In some embodiments, the insert size is 4.5-17, 6-17, 7-17, 8-17, 9-17, 10-17, 11-17, 12-17, 13-17, or 14-17, 15-17 or 16-17 kbp. In some embodiments, the insert size is 4.5-18, 6-18, 7-18, 8-18, 9-18, 10-18, 11-18, 12-18, 13-18, 14-18, 15-18, 16-18 or 17-18 kbp. In some embodiments, the insert size is 4.5-19, 6-19, 7-19, 8-19, 9-19, 10-19, 11-19, 12-19, 13-19, 14-19, 15-19, 16-19, 17-19, or 18-19 kbp. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, etc. In some embodiments, the the insert is a human sequence. In some embodiments, the insert is chimeric. In some embodiments, the insert is a multi-gene/multi-module therapeutic cassette. A multi-gene/multi-module therapeutic cassette referst to an insert or cassette having one or more than one receptor (e.g., synthetic receptors), other exogenous protein coding sequences, non-coding RNAs, transcriptional regulatory elements, and/or insulator sequences, etc.

In some embodiments, the nucleic acid sequence is inserted into the genome of the T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety. In some embodiments, the integration site is a GS94 (chr11:128340000-128350000) or GS12 (chr15:92830000-92840000) safe harbor site. In some embodiments, the integration site is any one of the sites selected from Table E. In some embodiments, the integration site is any one of the sites selected form the group consisting of GS88-GS120.

Examples of integration sites contemplated are provided in Table E.

TABLE E
sgRNA sequences

| | | SEQ | | | |
|---|---|---|---|---|---|
| sgRNA | | ID | sgRNA start | sgRNA Target | Integration |
| ID | sgRNA Sequence | NO: | coor GRCH38 | Loci | Site |
| sgRNA_ | GCACCTGAATACCACGCC | 161 | chr16: 88811818 | APRT | APRT |
| 1 | TG | | | | |
| sgRNA_ | CGCCTGCGATGTAGTCGA | 162 | chr16: 88811551 | APRT | APRT |
| 2 | TG | | | | |
| sgRNA_ | CAGGACGGGCGAGATGTC | 163 | chr16: 88811640 | APRT | APRT |
| 3 | CC | | | | |
| sgRNA_ | CTGAATCTTTGGAGTACCT | 164 | chr15: 44715425 | B2M | B2M |
| 4 | G | | | | |
| sgRNA_ | GGCCACGGAGCGAGACAT | 165 | chr15: 44711550 | B2M | B2M |
| 5 | CT | | | | |
| sgRNA_ | AAGTCAACTTCAATGTCG | 166 | chr15: 44715515 | B2M | B2M |
| 6 | GA | | | | |
| sgRNA_ | GCTTGGAGGCCTGATCAG | 167 | chr19: 36141111 | CAPNS1 | CAPNS1 |
| 7 | CG | | | | |
| sgRNA_ | CTTATCTCTTCGCAGCGAG | 168 | chr19: 36142301 | CAPNS1 | CAPNS1 |
| 8 | G | | | | |
| sgRNA_ | CACACATTACTCCAACATT | 169 | chr19: 36142676 | CAPNS1 | CAPNS1 |
| 9 | G | | | | |
| sgRNA_ | TTCCGCAAAATAGAGCCC | 170 | chr3: 105746019 | CBLB | CBLB |
| 10 | CA | | | | |
| sgRNA_ | TGCACAGAACTATCGTAC | 171 | chr3: 105751622 | CBLB | CBLB |
| 11 | CA | | | | |
| sgRNA_ | GCAATAAGACTCTTTAAA | 172 | chr3: 105853470 | CBLB | CBLB |
| 12 | GA | | | | |
| sgRNA_ | CAAAGAGATTACGAATGC | 173 | chr1: 116754658 | CD2 | CD2 |
| 13 | CT | | | | |
| sgRNA_ | CAAGGCACCCCAGGTTTC | 174 | chr1: 116754663 | CD2 | CD2 |
| 14 | CA | | | | |
| sgRNA_ | TTACGAATGCCTTGGAAA | 175 | chr1: 116754666 | CD2 | CD2 |
| 15 | CC | | | | |
| sgRNA_ | CAGAGACGCATCTGACCC | 176 | chr11: 118315540 | CD3E | CD3E |
| 16 | TC | | | | |
| sgRNA_ | CATGCAGTTCTCACACACT | 177 | chr11: 118313715 | CD3E | CD3E |
| 17 | G | | | | |
| sgRNA_ | GTGTGAGAACTGCATGGA | 178 | chr11: 118313715 | CD3E | CD3E |
| 18 | GA | | | | |
| sgRNA_ | TCTCATTTCAGGAAACCA | 179 | chr11: 118349748 | CD3G | CD3G |
| 19 | CT | | | | |
| sgRNA_ | AGTCATACACCTTAACCA | 180 | chr11: 118349754 | CD3G | CD3G |
| 20 | AG | | | | |
| sgRNA_ | TTCAAGGAAACCAGTTGA | 181 | chr11: 118352458 | CD3G | CD3G |
| 21 | GG | | | | |
| sgRNA_ | GAGCCTTGCCTGGAAATC | 182 | chr11: 61118177 | CD5 | CD5 |
| 22 | TG | | | | |
| sgRNA_ | AAGCGTCAAAAGTCTGCC | 183 | chr11: 61118324 | CD5 | CD5 |
| 23 | AG | | | | |
| sgRNA_ | CGTTCCAACTCGAAGTGC | 184 | chr11: 61118121 | CD5 | CD5 |
| 24 | CA | | | | |
| sgRNA_ | GAGCGACTGGGACACGGT | 185 | chr9: 136866246 | EDF1 | EDF1 |
| 25 | GA | | | | |
| sgRNA_ | GCTGCGCAAGAAGGGCCC | 186 | chr9: 136866211 | EDF1 | EDF1 |
| 26 | TA | | | | |
| sgRNA_ | TTGTTCTGGCCAGCAGCCC | 187 | chr9: 136863433 | EDF1 | EDF1 |
| 27 | C | | | | |
| sgRNA_ | CTTCCAGAGCCACATCAT | 188 | chr19: 48965791 | FTL | FTL |
| 28 | CG | | | | |
| sgRNA_ | GGGACTCACCAGAGAGAG | 189 | chr19: 48965601 | FTL | FTL |
| 29 | GT | | | | |
| sgRNA_ | CGGTCGAAATAGAAGCCC | 190 | chr19: 48965770 | FTL | FTL |
| 30 | TA | | | | |
| sgRNA_ | AAAAGGATATTGTGCAAC | 191 | chr10: 87933015 | PTEN | PTEN |
| 31 | TG | | | | |
| sgRNA_ | TGTGCATATTTATTACATC | 192 | chr10: 87933183 | PTEN | PTEN |
| 32 | G | | | | |
| sgRNA_ | TTTGTGAAGATCTTGACCA | 193 | chr10: 87933087 | PTEN | PTEN |
| 33 | A | | | | |
| sgRNA_ | TGTCATGCTGAACCGCATT | 194 | chr18: 12830972 | PTPN2 | PTPN2 |
| 34 | G | | | | |
| sgRNA_ | CCACTCTATGAGGATAGT | 195 | chr18: 12859219 | PTPN2 | PTPN2 |
| 35 | CA | | | | |
| sgRNA_ | TTGACATAGAAGAGGCAC | 196 | chr18: 12836828 | PTPN2 | PTPN2 |
| 36 | AA | | | | |
| sgRNA_ | GAGTACTACACTCAGCAG | 197 | chr12: 6952098 | PTPN6 | PTPN6 |
| 37 | CA | | | | |
| sgRNA_ | TCACGCACAAGAAACGTC | 198 | chr12: 6954872 | PTPN6 | PTPN6 |
| 38 | CA | | | | |
| sgRNA_ | AGGTCTCGGTGAAACCAC | 199 | chr12: 6951610 | PTPN6 | PTPN6 |
| 39 | CT | | | | |
| sgRNA_ | AGCATTATCCAAAGAGTC | 200 | chr1: 198696873 | PTPRC | PTPRC |
| 40 | CG | | | | |
| sgRNA_ | ATATTAATTCTTACCAGTG | 201 | chr1: 198692370 | PTPRC | PTPRC |
| 41 | G | | | | |
| sgRNA_ | AGCTTTAAATCAAGGTTC | 202 | chr1: 198756176 | PTPRC | PTPRC |
| 42 | AT | | | | |
| sgRNA_ | ATCCCGAGCCCTAAGGTG | 203 | chr11: 67436325 | PTPRCAP | PTPRCAP |
| 43 | CA | | | | |
| sgRNA_ | GGCAGCGCGGAGGACAGC | 204 | chr11: 67436285 | PTPRCAP | PTPRCAP |
| 44 | GT | | | | |
| sgRNA_ | CTCAGGGGGCTACTACCA | 205 | chr11: 67436170 | PTPRCAP | PTPRCAP |
| 45 | CC | | | | |
| sgRNA_ | GTCACCGACGAGACCAGA | 206 | chr5: 82277810 | RPS23 | RPS23 |
| 46 | AG | | | | |
| sgRNA_ | GTCGTGGACTTCGTACTGC | 207 | chr5: 82277843 | RPS23 | RPS23 |
| 47 | T | | | | |
| sgRNA_ | TAATTTTTAGGCAAGTGTC | 208 | chr5: 82277860 | RPS23 | RPS23 |
| 48 | G | | | | |
| sgRNA_ | TTAGCTGTTAGACTTGAAT | 209 | chr14: 51993810 | RTRAF | RTRAF |
| 49 | A | | | | |
| sgRNA_ | CGAGAGCCGTCAACTTGC | 210 | chr14: 51989652 | RTRAF | RTRAF |
| 50 | GT | | | | |
| sgRNA_ | CGGCTTCAACTGCAAAGG | 211 | chr14: 51989700 | RTRAF | RTRAF |
| 51 | TG | | | | |
| sgRNA_ | TATGAAAAAGCAGAGCGA | 212 | chr15: 43793025 | SERF2 | SERF2 |
| 52 | CT | | | | |
| sgRNA_ | TCTGGCGGGCGAGCTCAC | 213 | chr15: 43792989 | SERF2 | SERF2 |
| 53 | GC | | | | |
| sgRNA_ | CTCACGCTGGTTACCGCCT | 214 | chr15: 43792977 | SERF2 | SERF2 |
| 54 | A | | | | |
| sgRNA_ | AAAGATTACGAACTTCCC | 215 | chr12: 46207559 | SLC38A1 | SLC38A1 |
| 55 | TG | | | | |
| sgRNA_ | GTTAAAAACAGACATGCC | 216 | chr12: 46229232 | SLC38A1 | SLC38A1 |
| 56 | TA | | | | |
| sgRNA_ | ATGCCTAAGGAGGTTGTA | 217 | chr12: 46229246 | SLC38A1 | SLC38A1 |
| 57 | CC | | | | |
| sgRNA_ | CTCCAGGTATCCCATCGA | 218 | chr18: 47869418 | SMAD2 | SMAD2 |
| 58 | AA | | | | |
| sgRNA_ | CACCAAATACGATAGATC | 219 | chr18: 47870532 | SMAD2 | SMAD2 |
| 59 | AG | | | | |
| sgRNA_ | TGGCGGCGTGAATGGCAA | 220 | chr18: 47896729 | SMAD2 | SMAD2 |
| 60 | GA | | | | |
| sgRNA_ | TAGGATGGTAGCACACAA | 221 | chr16: 11255478 | SOCS1 | SOCS1 |
| 61 | CC | | | | |
| sgRNA_ | CAGCAGCAGAGCCCCGAC | 222 | chr16: 11255432 | SOCS1 | SOCS1 |
| 62 | GG | | | | |
| sgRNA_ | CGGCGTGCGAACGGAATG | 223 | chr16: 11255296 | SOCS1 | SOCS1 |
| 63 | TG | | | | |
| sgRNA_ | TATAGACGCTGCCCGACG | 224 | chr15: 40038895 | SRP14 | SRP14 |
| 64 | TC | | | | |
| sgRNA_ | TCCAAAGAAGGGTACTGT | 225 | chr15: 40038368 | SRP14 | SRP14 |
| 65 | GG | | | | |
| sgRNA_ | ACAGTACCCTTCTTTGGAA | 226 | chr15: 40038358 | SRP14 | SRP14 |
| 66 | T | | | | |
| sgRNA_ | GCGACGGGCGCATCTACG | 227 | chr12: 120469572 | SRSF9 | SRSF9 |
| 67 | TG | | | | |
| sgRNA_ | CCCGACCTCCATAAGTCCT | 228 | chr12: 120465700 | SRSF9 | SRSF9 |
| 68 | G | | | | |
| sgRNA_ | GGGGTCCTCGAAGCGCAC | 229 | chr12: 120469426 | SRSF9 | SRSF9 |
| 69 | GA | | | | |
| sgRNA_ | TGCTCTGTTTAGAAGATG | 230 | chr5: 32591641 | SUB1 | SUB1 |
| 70 | AC | | | | |
| sgRNA_ | ATATTCTTTTCTAGTTAAA | 231 | chr5: 32591566 | SUB1 | SUB1 |
| 71 | G | | | | |
| sgRNA_ | CCTGTAAAGAAACAAAAG | 232 | chr5: 32591614 | SUB1 | SUB1 |
| 72 | AC | | | | |
| sgRNA_ | TGGAGAAAGACGTAACTT | 233 | chr4: 105234315 | TET2 | TET2 |
| 73 | CG | | | | |
| sgRNA_ | TCTGCCCTGAGGTATGCG | 234 | chr4: 105234747 | TET2 | TET2 |
| 74 | AT | | | | |
| sgRNA_ | ATTCCGCTTGGTGAAAAC | 235 | chr4: 105235656 | TET2 | TET2 |
| 75 | GA | | | | |
| sgRNA_ | CAGGCACAATAGAAACAA | 236 | chr3: 114295571 | TIGIT | TIGIT |
| 76 | CG | | | | |
| sgRNA_ | CCATTTGTAATGCTGACTT | 237 | chr3: 114295700 | TIGIT | TIGIT |
| 77 | G | | | | |
| sgRNA_ | CTGGGTCACTTGTGCCGTG | 238 | chr3: 114295634 | TIGIT | TIGIT |
| 78 | G | | | | |
| sgRNA_ | GTCAGGGTTCTGGATATCT | 239 | chr14: 22547508 | TRAC | TRAC |
| 79 | G | | | | |
| sgRNA_ | TGGATTTAGAGTCTCTCAG | 240 | chr14: 22547541 | TRAC | TRAC |
| 80 | C | | | | |
| sgRNA_ | CTGCGGCTGTGGTCCAGC | 241 | chr14: 22550661 | TRAC | TRAC |
| 81 | TG | | | | |
| sgRNA_ | ACAAAACTGTGCTAGACA | 242 | chr14: 22547658 | TRAC | TRAC |
| 82 | TG | | | | |
| sgRNA_ | TTCTTCCCCAGCCCAGGTA | 243 | chr14: 22547778 | TRAC | TRAC |
| 83 | A | | | | |
| sgRNA_ | CGTCATGAGCAGATTAAA | 244 | chr14: 22550625 | TRAC | TRAC |
| 84 | CC | | | | |
| sgRNA_ | GAGAGCGCCTGCGACCCG | 245 | chr19: 58544980 | TRIM28 | TRIM28 |
| 85 | AG | | | | |
| sgRNA_ | CCAGCGGGTGAAGTACAC | 246 | chr19: 58544869 | TRIM28 | TRIM28 |
| 86 | CA | | | | |
| sgRNA_ | GGAGCGCTTTTCGCCGCC | 247 | chr19: 58544839 | TRIM28 | TRIM28 |
| 87 | AG | | | | |
| sgRNA_ | TGAGGCCTGGACCTTATG | 248 | chr10: 33134193 | chr10: 33130000 | desert 1 |
| 88 | CA | | | -33140000 | (GS88) |
| sgRNA_ | CCTGGTGGAGTGAACCAT | 249 | chr10: 33132917 | chr10: 33130000 | desert 1 |
| 89 | GA | | | -33140000 | (GS89) |
| sgRNA_ | CAAGCACTTAGGTTCCCCT | 250 | chr10: 33134633 | chr10: 33130000 | desert 1 |
| 90 | G | | | -33140000 | (GS90) |
| sgRNA_ | GGTCTCCCTACAATTCAGC | 251 | chr10: 72294568 | chr10: 72290000 | desert 2 |
| 91 | G | | | -72300000 | (GS91) |
| sgRNA_ | CACAGCGCGTGACTGCAA | 252 | chr10: 72298268 | chr10: 72290000 | desert 2 |
| 92 | TG | | | -72300000 | (GS92) |
| sgRNA_ | TCTGGGGCACCAATTCTA | 253 | chr10: 72292786 | chr10: 72290000 | desert 2 |
| 93 | GG | | | -72300000 | (GS93) |
| sgRNA_ | GAGCCATGCTTGGCTTAC | 254 | chr11: 128342576 | chr11: 12834000 | desert 3 |
| 94 | GA | | | 0-128350000 | (GS94) |
| sgRNA_ | GTACAAGTACTTATCTCAT | 255 | chr11: 128343592 | chr11: 12834000 | desert 3 |
| 95 | G | | | 0-128350000 | (GS95) |
| sgRNA_ | GAGATAACAACATAACAA | 256 | chr11: 128347170 | chr11: 12834000 | desert 3 |
| 96 | CA | | | 0-128350000 | (GS96) |
| sgRNA_ | CATATTCCATAGTCTTTGG | 257 | chr11: 65425000 | chr11: 65425000 | desert 4 |
| 97 | G | | | -65427000 | (GS97) |
| | | | | (NEAT1) | |
| sgRNA_ | CTGCCCCTTAGCAACTTAG | 258 | chr11: 65425507 | chr11: 65425000 | desert 4 |
| 98 | G | | | -65427000 | (GS98) |
| | | | | (NEAT1) | |
| sgRNA_ | TGTTTAAAAATATGTTGAC | 259 | chr11: 65426264 | chr11: 65425000 | desert 4 |
| 99 | A | | | -65427000 | (GS99) |
| | | | | (NEAT1) | |
| sgRNA_ | CCAGGAATGGAAACTCAC | 260 | chr15: 92830315 | chr15: 92830000 | desert 5 |
| 100 | GC | | | -92840000 | (GS100) |
| sgRNA_ | GAGGCCGCTGAATTAACC | 261 | chr15: 92831850 | chr15: 92830000 | desert 5 |
| 101 | CG | | | -92840000 | (GS101) |
| sgRNA_ | ATACACGCACACTTGCAG | 262 | chr15: 92831131 | chr15: 92830000 | desert 5 |
| 102 | AA | | | -92840000 | (GS102) |
| sgRNA_ | GAGCAGACAGAAACCCAG | 263 | chr16: 11225670 | chr16: 11220000 | desert 6 |
| 103 | GG | | | -11230000 | (GS103) |
| sgRNA_ | TGAGTCTCCAAACAGAAC | 264 | chr16: 11226284 | chr16: 11220000 | desert 6 |
| 104 | AG | | | -11230000 | (GS104) |
| sgRNA_ | TAATATCACTGACTTCACG | 265 | chr16: 11225029 | chr16: 11220000 | desert 6 |
| 105 | G | | | -11230000 | (GS105) |
| sgRNA_ | TACACACAATGTAAGCAG | 266 | chr2: 87467461 | chr2: 87460000- | desert 7 |
| 106 | CA | | | 87470000 | (GS106) |
| sgRNA_ | GGGAGCTCAATTCGAAAC | 267 | chr2: 87468809 | chr2: 87460000- | desert 7 |
| 107 | CA | | | 87470000 | (GS107) |
| sgRNA_ | TTGGACAGGTGAGACAGT | 268 | chr2: 87467001 | chr2: 87460000- | desert 7 |
| 108 | CG | | | 87470000 | (GS108) |
| sgRNA_ | AAGCTCACTCAGATAGTG | 269 | chr3: 186511316 | chr3: 186510000 | desert 8 |
| 109 | TG | | | -186520000 | (GS109) |
| sgRNA_ | CAGGAGAACCACCTTACA | 270 | chr3: 186515260 | chr3: 186510000 | desert 8 |
| 110 | CG | | | -186520000 | (GS110) |
| sgRNA_ | GGACAGACCCTGATTCAC | 271 | chr3: 186519655 | chr3: 186510000 | desert 8 |
| 111 | AA | | | -186520000 | (GS111) |
| sgRNA_ | ACATGGCAGTCTATGAAC | 272 | chr3: 59451154 | chr3: 59450000- | desert 9 |
| 112 | AG | | | 59460000 | (GS112) |
| sgRNA_ | CCTATAGAGAGTACTACT | 273 | chr3: 59456416 | chr3: 59450000- | desert 9 |
| 113 | TG | | | 59460000 | (GS113) |
| sgRNA_ | CCAACCGGGTCTTCATTAC | 274 | chr3: 59457029 | chr3: 59450000- | desert 9 |
| 114 | G | | | 59460000 | (GS114) |
| sgRNA_ | TCAAGCGTAGAGTTCCGA | 275 | chr8: 127993006 | chr8: 127980000 | desert 10 |
| 115 | GT | | | -128000000 | (GS115) |
| sgRNA_ | TCATGCAATTATGGACCC | 276 | chr8: 127994663 | chr8: 127980000 | desert 10 |
| 116 | AG | | | -128000000 | (GS116) |
| sgRNA_ | CGGGAAAGTGACTGGCCA | 277 | chr8: 127996766 | chr8: 127980000 | desert 10 |
| 117 | TG | | | -128000000 | (GS117) |
| sgRNA_ | TGAGATTGAAATCAAATC | 278 | chr9: 7974159 | chr9: 7970000- | desert 11 |
| 118 | GG | | | 7980000 | (GS118) |
| sgRNA_ | TATGCAATATTCATCACGC | 279 | chr9: 7977914 | chr9: 7970000- | desert 11 |
| 119 | G | | | 7980000 | (GS119) |
| sgRNA_ | AATGTGTTAAATCAAATG | 280 | chr9: 7976895 | chr9: 7970000- | desert 11 |
| 120 | CA | | | 7980000 | (GS120) |

### CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540(7631), 144-149, the relevant disclosures of which are herein incorporated in their entirety).

Methods for conducing gene editing using CRISPR-Cas approaches are known to those of ordinary skill in the art. (See, for example, US Application Nos. U.S. Ser. No. 16/312,676, U.S. Ser. No. 15/303,722, and U.S. Ser. No. 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount. The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of an immune-related disorder, such as cancer. The appropriate dosage of the immune cell comprising the system may be determined based on the type of immune-related disorder to be treated, the type of the immune cell comprising the system, the severity and course of the cancer, the clinical condition of the individual, the individual's clinical history and response to the treatment, and the discretion of the attending physician.

### Pharmaceutical Compositions

The engineered recombinant cells provided herein can be administered as part of a pharmaceutical compositions. These compositions can comprise, in addition to one or more of the recombinant cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the system or cell compositions described herein along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Screen and Characterization of Novel Transmembrane and Juxtamembrane Domains

### Design and Construction of Combinatorial Prime Receptor Library

A library of approximately 6200 TMDs and 1250 JMDs were identified from the Uniprot protein database and codon optimized for mammalian expression. The 6200 TMDs were combined with two designated JMDs (NOTCH1 and NOTCH2) to create a TMD library of 12400 variants and the 1250 JMDs were combined with 8 designated TMDs to create a JMD library of 10000 variants. These variants were ordered as oligo libraries from Twist Biosciences, amplified with barcoded primers, and cloned into an intermediate cloning vector to make variants with an CD19 binder (FMC63) and an EGFR reporter gene. The intermediate library was transformed into DH5a E. coli (NEB, Cat: C2987I), plated on 245×245 mm QTrays (Fisher Scientific, #50-842-527), and scraped according to desired library size. Amplicon NGS was performed to link prime receptor variants with specific barcodes. A CAR constant fragment was then inserted into the intermediate library plasmids by restriction cloning and the full library was transformed into DH5a E. coli (NEB, Cat: C2987I). The final plasmid library was verified with Amplicon NGS to confirm expected distribution of variants.

Non-Viral Integration of Library into Primary Human T Cells

Fresh human T cells (CD4+CD8) were isolated via leukapheresis of fresh human blood using the AutoMACs system. T cells were activated using CTS Dynabeads (ThermoFisher, Cat: 40203D) at a 1:1 bead to cell ratio and a T cell concentration of 1×10⁶ cells/mL in T175 flasks. T cells were then debeaded and electroporated with plasmid library DNA mixed with ribonucleoprotein (RNP; Cas9 recombinant protein and single guide RNA) using the Lonza 384-well Nucleofector System (Cat: AAU-1001). Electroporated T cells were then recovered in TexMACs (Miltenyi Biotec, Cat: 170-076-309)+3% Human AB serum (Gemini, Cat: 100-512)+12.5 ng/mL human IL-7 (Mitenyi Biotec, Cat: 130-095-364) +12.5 ng/mL human IL-15 (Miltenyi Biotec, Cat: 130-095-766). Cells were cultured in G-rex 6 well plates (Wilson Wolf, Cat: 80240M) for 5 days post-EP with media supplementation every 48 hours. The TMD and JMD libraries were electroporated into 200×10⁶ T cells from two different donors (total 400×10⁶ for each library).

### Pooled Screening of Prime Receptor Library

Library T cells were stained with anti-EGFR APC antibody (BioLegend, 352905), mixed with counting beads (Life Technologies, Cat: C36995), and measured using an Attune N×T flow cytometer. The editing percentage was estimated based on EGFR+ signal. Library T cells were then co-cultured with K562 (parental cells), K562-ALPG low, or K562-ALPG high for 24 hours in human T cell media: TexMACS+3% Human AB serum+15 μg/mL Gentamicin (Gibco, Cat: 15-750-078). Co-cultures were set up at an effector:target ratio of 1:3 (1 edited T cell: 3 tumor cells) with 5×10⁶ edited T cells in each co-culture sample for each donor. Samples were then pelleted at 300×g for 5 min for downstream NGS analysis. The library gene constructs were designed to have a unique barcode immediately downstream of the CAR such that prime receptor engagement of target antigen would induce CAR and barcode on the same mRNA transcript. Amplicon NGS sequencing could then be performed to count barcodes and directly link prime receptors to their induction capacity.

### NGS Readout+Barcode Enrichment Analysis

RNA was isolated from library co-culture samples using an RNeasy Plus Midi Kit (Qiagen, Cat: 75144). Sequencing libraries were prepared by performing reverse transcriptase using an oligo dT and a template switching oligo (TSO) primer set, amplifying cDNA with gene specific primers, and then adding i5 and i7 sequences with a final index PCR. Amplicons containing a 26 bp variant-specific barcode were deep sequenced using an Illumina NovaSeq.

### Computational Analysis

Barcode read FASTQ files were filtered through the clustering pipeline “Starcode” using a Levenshtein distance of 2. The barcode clusters were then assigned to their corresponding variant using a barcode linkage list and the totals for each variant were counted. The variant counts were then analyzed with the differential expression package “EdgeR” to calculate enrichment. Statistically significant enrichment in K562-ALPGhi/plasmid, K562-ALPGhi/T cells only, K562-ALPGhi/K562-parental, K562-ALPGlow/plasmid, K562-ALPGlow/T cells only, K562-ALPGlow/K562-parental comparisons were necessary to identify variants to select for further analysis. Selected variants were further filtered by removing those with significant enrichment in K562-parental/T cells only and T cells only/plasmid comparisons as indications of non-specific basal activity. Based on these criteria, 171 variants were chosen for downstream arrayed analysis.

### Arrayed Screening Setup for Head to Head Comparison

The top 171 variants were cloned into acceptor vectors with an ALPG/P scFv binder (H17E2) and an HNF1a-p65 or Gal4-VP64 transcription factor. The vectors also expressed a constant 4-1BBz CAR with an N-terminal FLAG-tag. Primary T cells were isolated and activated for 48 hr with anti-CD3/anti-CD28 dynabeads at a 1:1 ratio. T cells were engineered by non-viral transfection of CRISPR/Cas9 and template DNA for each variant in 384 well format. Cell counts, prime receptor expression, and knock-in frequency were determined five days after transfection by analysis of myc tag expression on the N-terminus of the prime receptor with a Sartorius iQue® 3. Each variant was normalized to an equivalent cell concentration and % prime receptor+ by adding the appropriate amount of non-edited T cells and human T cell medium.

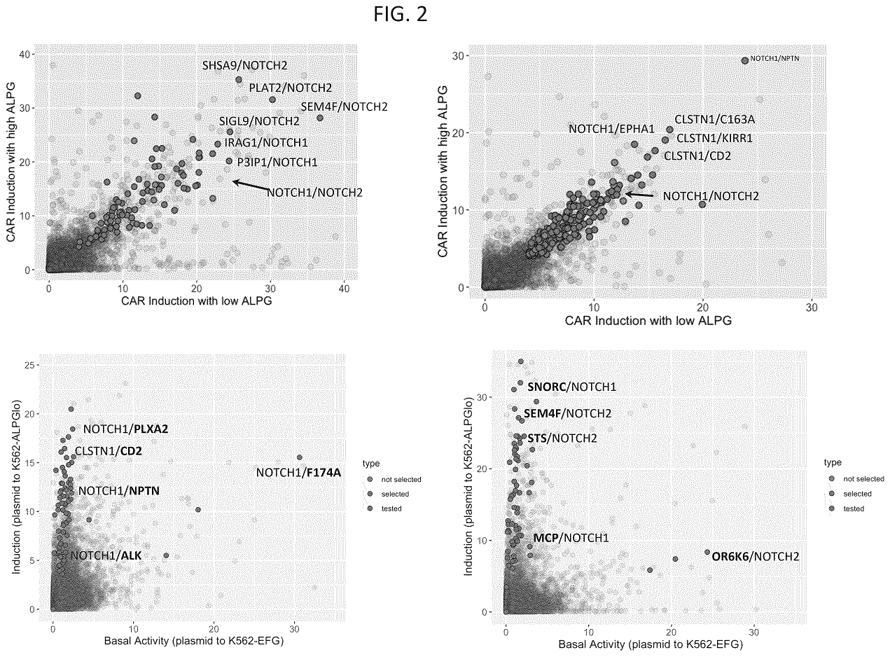

Selected hits from the screening assay are shown in FIG. 2.

### CAR Induction Capacity

To characterize CAR control and induction levels for each priming receptor variant, 1.5×10⁴ prime+ T cells were co-cultured at a 1:3 ratio with K562-ALPG+ or K562-parental K562s in a U-bottom 96-well TC plate. 48 hr later, the co-cultures were stained with anti-FLAG BV421 (BioLegend, Cat: 637322) and measured by flow cytometry on a Sartorius iQue® 3. Analysis of CAR MFI and % CAR positive cells was done using FlowJo™ software. A priming receptor with a NOTCH1_extendedNotch1 TMD/JMD was used as a positive control. Priming receptors with OR6K6-NOTCH2 or NOTCH1-F174A TMD/JMDs were predicted to be non-controllers.

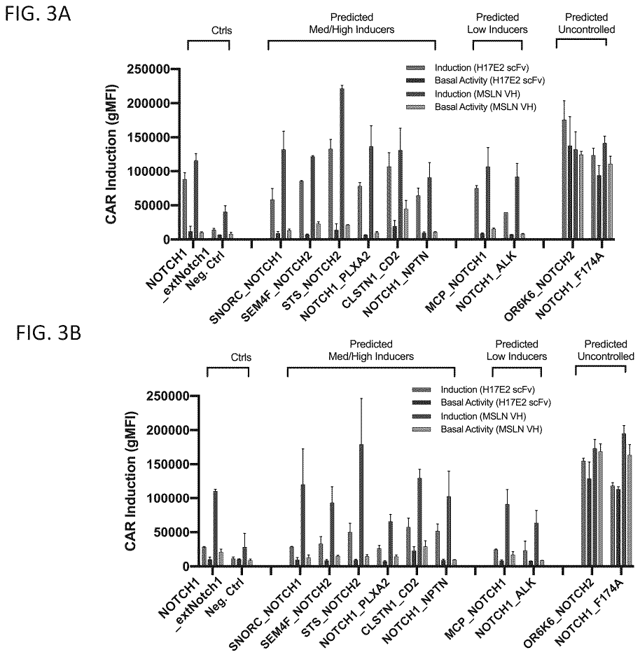

The induction results from an initial panel of 12 prime receptor variants are shown in FIGS. 3A and 3B with primary T cells originating from two different donors (FIG. 3A and FIG. 3B, respectively). The NOTCH1_PLXA2, NOTCH1_NPTN, and NOTCH1_ALK show slightly lower CAR induction after co-culture with K562-ALPGlow and K562-ALPGhi compared to the NOTCH1_extendedNotch1 (NOTCH1-extNOTCH1). These variants also show slightly lower basal activity after co-culture with K562-parental cells. Predicted prime receptors behavior was reproducible in both binder types. The STS_NOTCH2, NOTCH1_PLXA2, and SEM4F_NOTCH2 prime receptors showed similar induction levels as the NOTCH1-extNOTCH1 control (SEQ ID NO: 301 and 131). STS_NOTCH2 showed a higher CAR induction than the other prime receptors. Higher basal levels of CAR expression than expected were observed with the CLSTN1_CD2 prime receptor. The NOTCH1_ALK prime receptor has a lower induction level with very minimal basal activity. The OR6K6_NOTCH2 and NOTCH1_F174A primer receptors had high basal activity.

### Cytotoxicity Assay

For determining the circuit function, circuit sensitivity, and priming receptor antigen cytotoxicity dependance, an E:T ratio of 1:1 was held constant while the ratio of target cells (ALPG/MSLN:MSLN) was changed. 1.5×10⁴ prime receptor expressing primary T cells were co-cultured at a 1:1 ratio with 100% K562-ALPG/MSLN cells, 2.5% K562-ALPG/MSLN and 97.5% K562-MSLN cells, or 0% K562-ALPG/MSLN (e.g., 100% K562-MSLN) Luciferase-expressing cells in U-bottom 96-well TC plates. 48 hr later, the co-cultures were lysed and analyzed by a luciferase reporter assay with a Pherastar microplate reader.

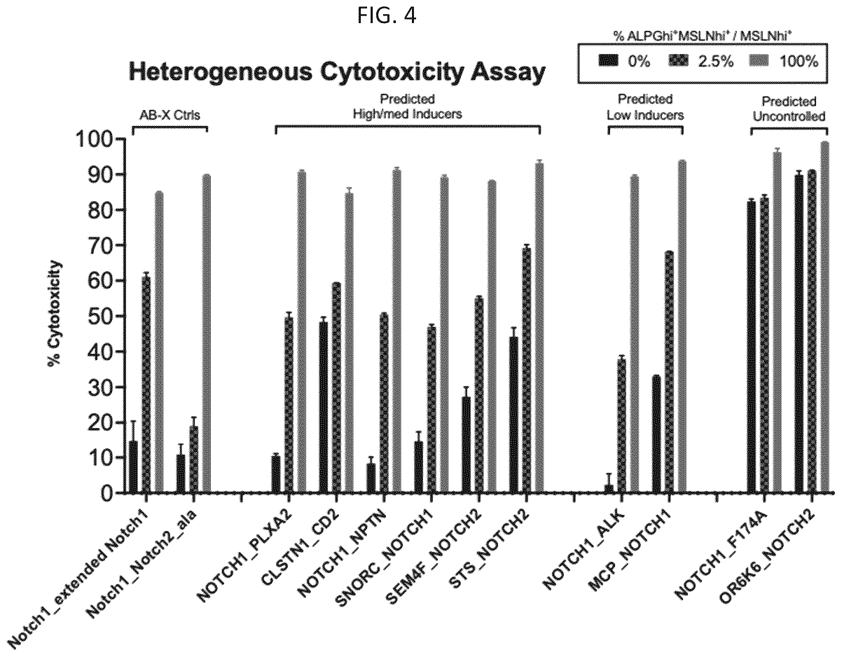

Cytotoxicity results from an initial panel of 12 prime receptor variants are shown in FIG. 4. Cytotoxicity in the 0% condition represents antigen-independent killing of MSLN+K562s. All of the pooled screen candidates showed higher sensitivity than the alanine mutant control. The NOTCH1_ALK variant demonstrated controlled killing with moderate cytotoxicity in the 2.5% condition and low killing in the 0% condition. NOTCH1_ALK had the lowest levels of prime receptors independent cytotoxicity

This cytotoxicity readout enables better resolution between controlled and uncontrolled priming receptor candidates.

### Example 2: Screen and Characterization of Additional Novel Transmembrane and Juxtamembrane Domains

### Arrayed Screening Assay

A further arrayed screen of top prime receptor candidates nominated from the pooled screen from Example 1 was performed, as described above. Briefly, variants were cloned into acceptor vectors with a HNF1a-p65 or Gal4-VP64 transcription factor and a constant 4-1BBz CAR with an N-terminal FLAG-tag. Primary T cells were isolated and activated for 48 hr with anti-CD3/anti-CD28 dynabeads at a 1:1 ratio. T cells were engineered by non-viral transfection of CRISPR/Cas9 and template DNA for each variant in 384 well format. Expression of prime receptors was measured by flow cytometry after co-culture with K562 parental cells (K562-EFG). Induction of CAR was measured by flow cytometry after co-culture with K562-ALPGlo cells.

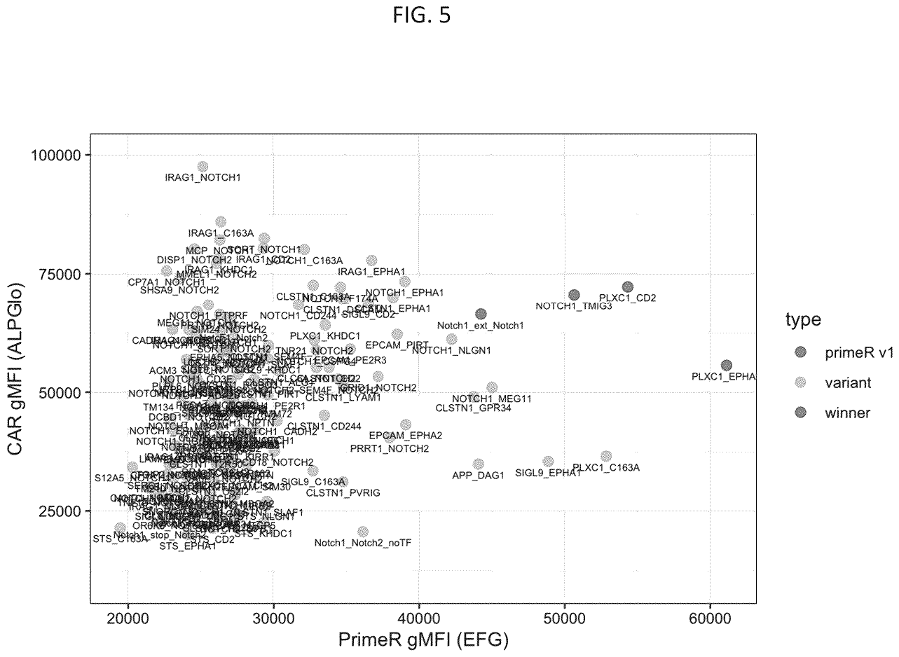

The additional screening results are shown in FIG. 5. Three variants were selected for prime receptor characterization based on increased prime receptor expression over the control prime receptor with NOTCH1-extNOTCH1 transmembrane and juxtamembrane domains: NOTCH1_TMIG3, PLXC1_CD2, PLXC1_EPHA1.

### CAR Induction Capacity

CAR control and induction levels for each priming receptor variant were performed as described above in Example 1.

### Prime Receptor to CAR Conversion (Turnover)

Turnover was calculated as the percentage of CAR expressed after a 72 hour co-culture with K562-ALPGlo cells divided by percentage of PrimeR expressed after a 72 hour co-culture with K562-EFG cells. This was a measure of conversion efficiency of prime receptor to CAR expression. Induced CAR gMFI was measured via flow cytometry after a 72 hour co-culture with K562-ALPGlo cells and was calculated as the geometric mean of FLAG fluorescence.

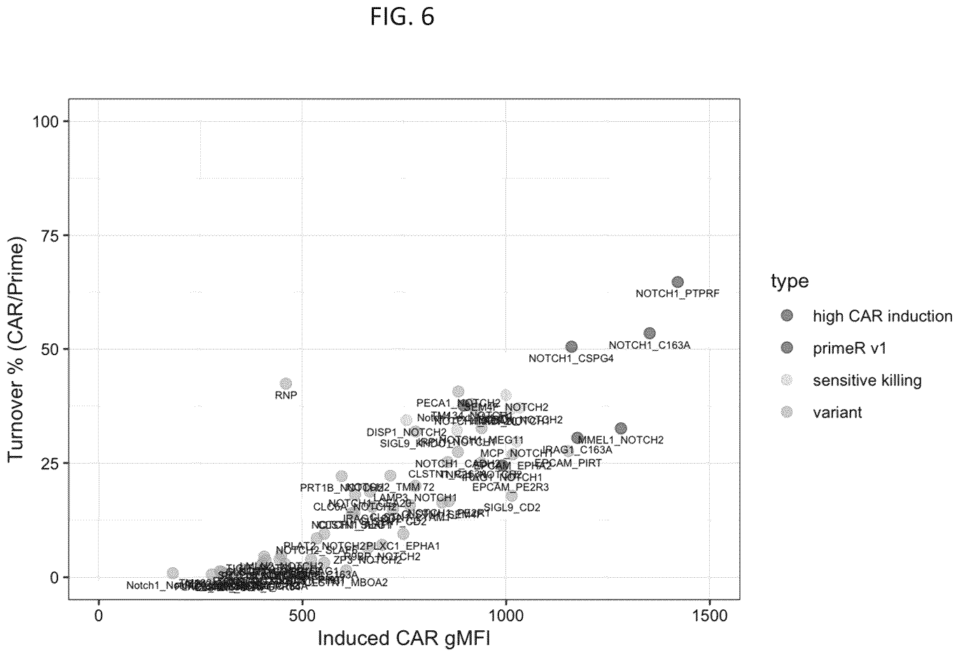

The CAR conversion results are shown in FIG. 6. Five variants were selected with a high CAR conversion and induced CAR gMFI: IRAG1_C163A, MMEL1_NOTCH2, NOTCH1_CSPG4, NOTCH1_C163A, NOTCH1_PTPRF.

### CAR Induction and Cytotoxicity Assay

CAR induction assay: 1×10⁴ prime receptor expressing primary T cells were co-cultured at a 1:3 (E:T) ratio with K562-ALPG cells in U-bottom 96-well TC plates to assess CAR induction. Induced CAR gMFI was measured via flow cytometry after a 72 hour co-culture with the target cells.

Cytotoxicity assay: 1×10⁴ prime receptor expressing primary T cells were co-cultured at a 1:1 (E:T) ratio in a heterogeneous target cell condition (2.5% ALPG/MSLN cells, 97.5% MSLN only cells) in U-bottom 96-well TC plates to assess cytotoxicity. 48 hr later, the co-cultures were lysed and analyzed by a luciferase reporter assay with a Pherastar microplate reader.

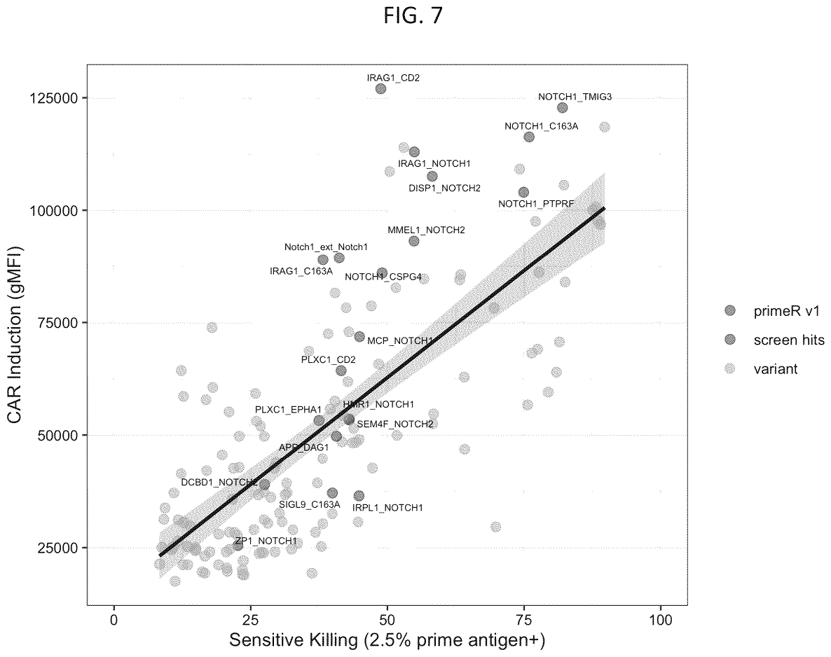

The CAR induction and cytotoxicity results are shown in FIG. 7. Hits with variable CAR induction were chosen for further analysis. High inducers that showed sensitive killing included NOTCH1_TMIG3, IRAG1_CD2, NOTCH1_C163A, IRAG1_NOTCH1, DISP1_NOTCH2, and NOTCH1_PTPRF

### CAR Fidelity

Leaky killing was assessed by measuring luciferase from target cells (K562-MSLN) after 48 hr co-culture. 1×10⁴ prime receptor expressing primary T cells were co-cultured in U-bottom 96-well TC plates at a 1:1 (E:T) ratio with K562-MSLN target cells for 48 hrs. The co-cultures were lysed and analyzed by a luciferase reporter assay with a Pherastar microplate reader.

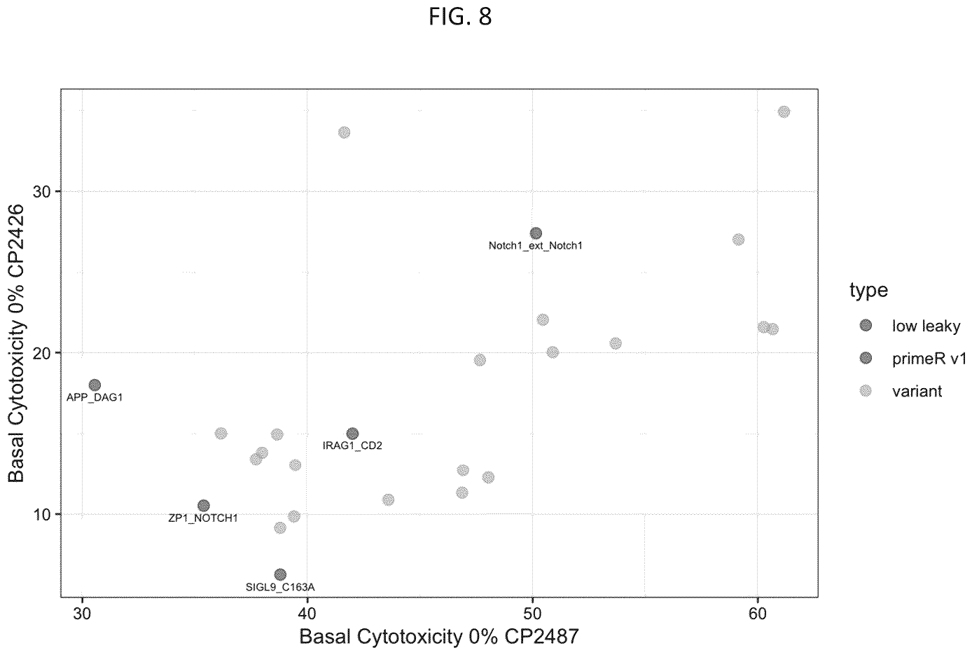

The fidelity results are shown in FIG. 8. Four variants had particularly high fidelity (low off-target killing) in two different T cell donors: APP_DAG1, ZP1_NOTCH1, SIGL9_C163A, IRAG1_CD2.

### Cytotoxicity Comparison Assay of Selected Variants

A selection of the novel TIMD and JMD candidates identified from the initial arrayed screen were chosen for further head to head comparisons. 1×10⁴ prime receptor expressing primary T cells were co-cultured at a 1:1 ratio with 100% K562-ALPG/MSLN cells, 5% K562-ALPG/MSLN and 95% K562-MSLN cells, or 0% K562-ALPG/MSLN (e.g., 100% K562-MSLN) Luciferase-expressing cells in U-bottom 96-well TC plates. 48 hr later, the co-cultures were lysed and analyzed by a luciferase reporter assay with a Pherastar microplate reader.

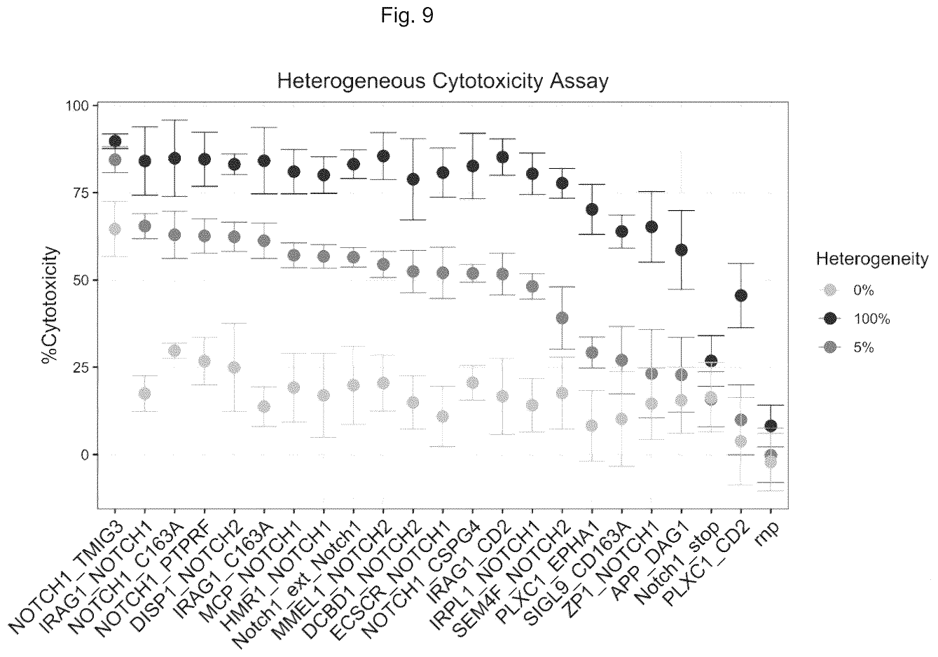

Varying levels of heterogeneous killing was observed by the selected prime receptors (FIG. 9). NOTCH1_TMIG3 demonstrated the highest level of heterogeneous killing in the 5% K562-ALPG/MSLN condition, but also demonstrated leaky killing in the 0% K562-ALPG/MSLN condition.

### CAR Induction Vs Basal Killing

CAR induction assay: 1×10⁴ prime receptor expressing primary T cells were co-cultured at a 1:3 (E:T) ratio with K562-ALPG cells in U-bottom 96-well TC plates to assess CAR induction. Induced CAR gMFI was measured via flow cytometry after a 72 hour co-culture with the target cells.

Cytotoxicity assay: 1×10⁴ prime receptor expressing primary T cells were co-cultured at a 1:1 (E:T) ratio in a heterogeneous target cell condition (2.5% ALPG/MSLN cells, 97.5% MSLN only cells) in U-bottom 96-well TC plates to assess cytotoxicity. 48 hr later, the co-cultures were lysed and analyzed by a luciferase reporter assay with a Pherastar microplate reader.

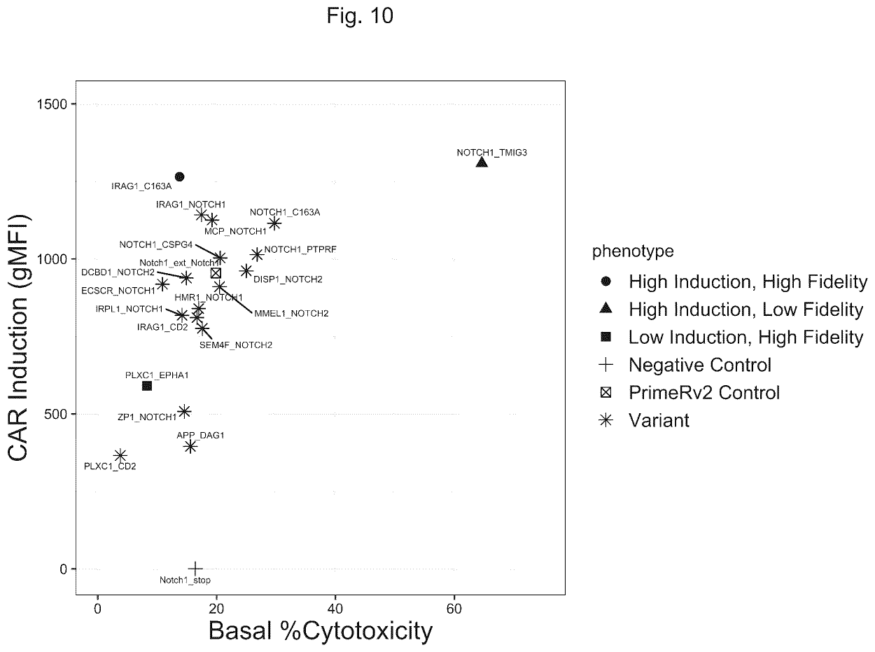

The results are shown in FIG. 10. Three different phenotypes were observed from the small subset of variants in arrayed screen 2. PLXC1_EPHA1 demonstrated very high fidelity (low basal cytotoxicity) and low induction. IRAG1_C163A demonstrated low basal cytotoxicity and high CAR induction. NOTCH1_TMIG3 demonstrated high basal cytotoxicity and high CAR induction. These three variants were selected for downstream characterization.

### Live Cell Killing Assay

Live cell killing was measured over time to compare killing kinetics of prime receptor with IRAG1_C163A and PLXC1_EPHA1 TMD/JMDs against a Notch1_extNotch1 prime receptor (positive control). Unedited T cells (RNP) were used as an additional control. Two target cell lines were used: RPMI-ALPG/MSLN and RPMI-MSLNhi. Prime receptor expressing primary T cells were co-cultured at a 1:1 (E:T) ratio with 25×10⁴ RPMI-8226-EFG-ALPG/MSLN (vLo/Hi) target cells to assess on target killing. Basal killing was also assessed by incubating 25×10⁴ RPMI-8226-EFG-MSLN (Hi) target cells with prime receptor expressing primary T cells at a 1:3 E:T ratio. Cell killing was assessed by GFP intensity over time as measured by Incucyte.

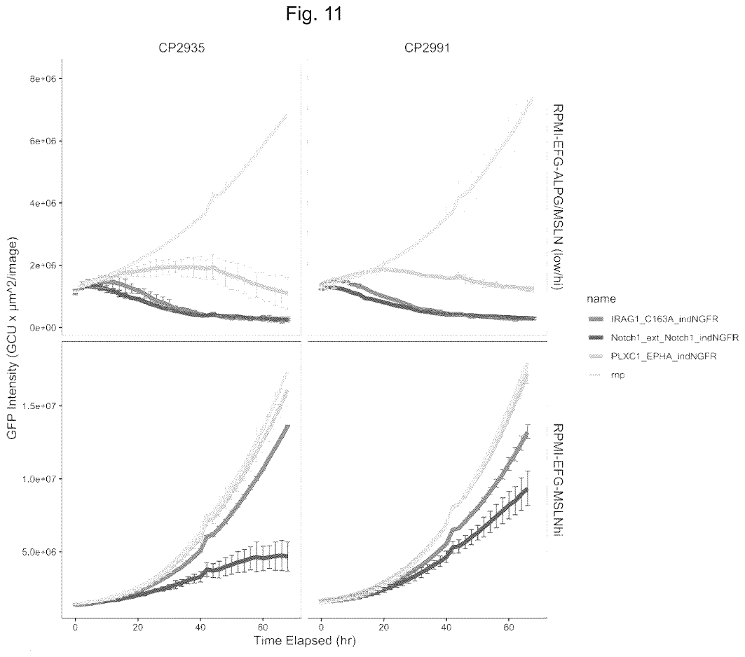

As shown in FIG. 11, the IRAG1_C163A prime receptor had comparable on-target killing of the target cells as the Notch1_extNotch1 prime receptor against the RPMI-ALPG/MSLN cells. The IRAG1_C163A prime receptor also demonstrated higher fidelity in the off target condition (RPMI-MSLNhi cells). The PLXC1_EPHA1 prime receptor prevented tumor cell proliferation but had lower on-target killing as compared to the Notch1_extNotch1 prime receptor. The PLXC1_EPHA1 prime receptor demonstrated remarkable fidelity by matching the killing of unedited T cells (RNP control).

Live Cell Killing with Constitutive Receptor Assay

The live cell killing assay was repeated with T cells that also expressed a large constitutive protein downstream of the prime receptor (truncated EGFR, EGFRt). The assay was performed as described above.

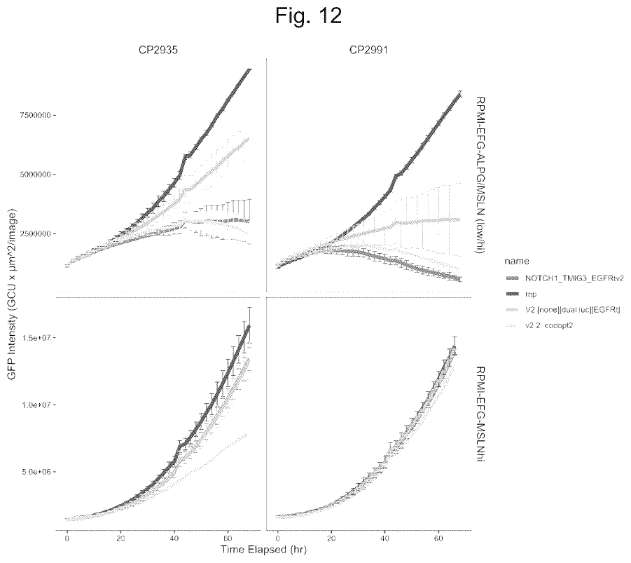

Live cell killing was measured over time to compare killing kinetics of prime receptor variants expressing a large constitutive protein downstream of the prime receptor. The Notch1_extNotch1 prime receptor was used as a benchmark control. Without wishing to be bound by theory, the inclusion of a large constitutively expressed protein has been shown to decrease prime receptor expression and CAR induction. As shown in FIG. 12, the highly inducing NOTCH1_TMIG3 prime receptor demonstrated improved on-target killing compared to the Notch1_extNotch1 prime receptor benchmark without a corresponding increase in leaky killing.

A summary of the additional characterization of the novel prime receptors is provided in Table 1.

TABLE 1

| | PrimeR | CAR | CAR |
|---|---|---|---|
| TMD_JMD Toolbox | Expression | Induction | Leakiness |
| NOTCH1_TMIG3 | High | High | High |
| IRAG1_C163A | Medium/Low | High | Low |
| PLXC1_EPHA | Very High | Low | Very Low |
| NOTCH1_extNOTCH1 | Medium | Medium | Medium/Low |

In sum, the pooled screening yielded more than 20 TMD/JMD pairs that outperformed the fidelity of the benchmark Notch-like prime receptor (Notch1_extNotch1). These new prime receptor pairs induced CAR at varying levels, which may form a toolbox for future genetic circuit tuning. Arrayed screening revealed multiple new TMD/JMD pairs with higher fidelity and comparable killing to the benchmark receptor. A novel TMD/JMD pair, PLXC1_EPHA1, was found with exceptional specificity and slightly decreased CAR induction capacity. IRAG1_C163A also showed high specificity and comparable CAR induction as the Notch1_extNotch1 benchmark control.

Without wishing to be bound by theory, the addition of a downstream, constitutively expressed protein can boost T cell survival, but can also decrease the expression of other proteins in the circuit. Multiple novel JMDs from the screen increased CAR induction and cytotoxicity capacity over benchmark levels in circuits with this additional constitutive protein.

Without wishing to be bound by theory, these novel prime receptor TMD/JMD pairs may allow for genetic circuit tuning in future cell therapy products. The high fidelity prime receptors can increase the safety of these products by limiting off target killing and could be used for inducing payloads that may be toxic if delivered systemically but safe and potent if delivered locally. Without wishing to be bound by theory, the high inducing prime receptors can allow for larger gene circuits with additional protein components, such as constitutive receptors that promote T cell survival and proliferation.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ | | |
|---|---|---|
| ID | | |
| NO | Name | Sequence |
| 1 | STS TMD | LGVFFSLLFLAALILTLFLGEL |
| 2 | SNORC TMD | IAAIVIAALLATCVVLALVVV |
| 3 | SEM4F TMD | GAGLAGFFLGILAASLTLILIG |
| 4 | MCP TMD | VWVIAVIVIAIVVGVAVICVVPY |
| 5 | PLAT2 TMD | VTTVGVAAGLLAAASLVGILLA |
| 6 | MMEL1 TMD | GLLLLLLLVTAALVALVALGVLYAD |
| 7 | PLXC1 TMD | WYFLIVLPVLLVIVIFAAVGV |
| 8 | CLSTN1 TMD | ATVVIVVCVSFLVFMIILGVF |
| 9 | CSTN2 TMD | IATVVIIISVCMLVFVVAMGV |
| 10 | SIGL9 TMD | VVGGAGATALVFLSFCVIFVV |
| 11 | IRAG1 TMD | WQVIWMMAAVMLVLTVVLGLY |
| 12 | TIGIT TMD | LLGAMAATLVVICTAVIVVVALT |
| 13 | TNR21 TMD | LPWMIVLFLLLVLVVIVVCSI |
| 14 | ZP3 TMD | VLLGVGLAVVVSLTLTAVILVLT |
| 15 | SUSD4 TMD | WKIVAFTATSVLLVLLLVILA |
| 16 | DCBD1 TMD | AIPLVLLVVLVFAGMGIFAAF |
| 17 | HMR1 TMD | KAVSGSIVLVIVLAGVGVLVW |
| 18 | DISP1 TMD | VVVLGMCTMFIVVCALVGVLV |
| 19 | IRPL1 TMD | VELAGGLGAILLLLVCLVTIY |
| 20 | ECSCR TMD | FGVISFIVILVVVVIILVGVV |
| 21 | NOTCH1 TMD | FMYVAAAAFVLLFFVGCGVLL |
| 22 | PLXA2 JMD | KRKSR |
| 23 | NPTN JMD | KRKR |
| 24 | ALK JMD | RRKH |
| 25 | DSCAM JMD | RRRRREQRLKRLRDAK |
| 26 | CD2 JMD | KRKKQRSRR |
| 27 | EPHA1 JMD | RSRRAQRQRQQRQRDR |
| 28 | ERMAP JMD | KQRRAKEK |
| 29 | ERBB2 JMD | KRRQQKIRK |
| 30 | CEA20 JMD | RNARRPSRK |
| 31 | KIRR1 JMD | RRRKGSRK |
| 32 | C163A JMD | SKKRRQRQR |
| 33 | PTPRF JMD | SKRKRTH |
| 34 | CSPG4 JMD | SRKRNKTGKH |
| 35 | SLAF1 JMD | SRRRGKTNH |
| 36 | TMIG3 JMD | SKRRRSQRNRR |
| 37 | NOTCH1 JMD | SRKRRR |
| 38 | NOTCH2 JMD | KRKRKH |
| 39 | Ext-NOTCH1 JMD | RKRRRQHGQLWFPEGFKVSEASKKKRREPLGEDSVGLKPLKNA |
| 40 | DCBD1_NOTCH2 | AIPLVLLVVLVFAGMGIFAAFKRKRKH |
| | amino acid | |
| 41 | IRAG1_C163A | WQVIWMMAAVMLVLTVVLGLYKKRRQRQR |
| | amino acid | |
| 42 | HMR1_NOTCH1 | KAVSGSIVLVIVLAGVGVLVWSRKRRR |
| | amino acid | |
| 43 | MCP_NOTCH1 | VWVIAVIVIAIVVGVAVICVVPYSRKRRR |
| | amino acid | |
| 44 | DISP1_NOTCH2 | VVVLGMCTMFIVVCALVGVLVKRKRKH |
| | amino acid | |
| 45 | IRPL1_NOTCH1 | VELAGGLGAILLLLVCLVTIYSRKRRR |
| | amino acid | |
| 46 | ECSCR_NOTCH1 | FGVISFIVILVVVVIILVGVVSRKRRR |
| | amino acid | |
| 47 | NOTCH1_PTPRF | FMYVAAAAFVLLFFVGCGVLLSKRKRTH |
| | amino acid | |
| 48 | NOTCH1_CSPG4 | FMYVAAAAFVLLFFVGCGVLLSRKRNKTGKH |
| | amino acid | |
| 49 | SEM4F_NOTCH2 | GAGLAGFFLGILAASLTLILIGKRKRKH |
| | amino acid | |
| 50 | IRAG1_CD2_amino | WQVIWMMAAVMLVLTVVLGLYKRKKQRSRR |
| | acid | |
| 51 | SIGL9_CD2_amino | VVGGAGATALVFLSFCVIFVVKRKKQRSRR |
| | acid | |
| 52 | IRAG1_NOTCH1 | WQVIWMMAAVMLVLTVVLGLYSRKRRR |
| | amino acid | |
| 53 | NOTCH1_SLAF1 | FMYVAAAAFVLLFFVGCGVLLSRRRGKTNH |
| | amino acid | |
| 54 | NOTCH1_TMIG3 | FMYVAAAAFVLLFFVGCGVLLSKRRRSQRNRR |
| | amino acid | |
| 55 | NOTCH1_C163A | FMYVAAAAFVLLFFVGCGVLLSKKRRQRQR |
| | amino acid | |
| 56 | PLXC1_EPHA1 | WYFLIVLPVLLVIVIFAAVGVRSRRAQRQRQQRQRDR |
| | amino acid | |
| 57 | Notch1_ext_Notch1 | FMYVAAAAFVLLFFVGCGVLLSRKRRRQHGQLWFPEGFKVSEASKKKRREPLGEDSVG |
| | amino acid | LKPLKNA |
| 58 | DCBD1_NOTCH2 | GCTATACCCTTAGTACTACTCGTTGTTCTTGTCTTTGCCGGAATGGGGATATTCGCAG |
| | nucleic acid | CCTTCAAACGCAAACGAAAACAC |
| 59 | IRAG1_C163A | TGGCAAGTGATCTGGATGATGGCCGCTGTGATGCTCGTGCTGACAGTCGTCCTGGGCC |
| | nucleic acid | TCTACAAAAAGAGGAGACAGCGCCAGCGG |
| 60 | HMR1_NOTCH1 | AAAGCTGTTTCCGGGTCAATAGTTCTTGTTATAGTTTTAGCCGGAGTCGGCGTACTCG |
| | nucleic acid | TCTGGAGCAGAAAACGCCGGCGA |
| 61 | MCP_NOTCH1 | GTTTGGGTTATAGCCGTTATAGTTATAGCAATAGTCGTCGGCGTCGCTGTTATATGTG |
| | nucleic acid | TAGTACCCTACAGCCGAAAAAGACGGCGC |
| 62 | DISP1_NOTCH2 | GTTGTTGTTCTCGGGATGTGCACAATGTTTATAGTTGTTTGTGCCTTAGTCGGCGTAC |
| | nucleic acid | TTGTCAAACGAAAACGCAAACAC |
| 63 | IRPL1_NOTCH1 | GTAGAGTTAGCCGGAGGGCTAGGCGCAATACTTCTCCTCCTTGTTTGCCTTGTCACTA |
| | nucleic acid | TATACAGCAGAAAACGACGCCGG |
| 64 | ECSCR_NOTCH1 | TTCGGCGTCATAAGTTTTATAGTAATACTCGTTGTTGTCGTCATAATACTTGTAGGAG |
| | nucleic acid | TTGTTAGCCGGAAACGCAGACGA |
| 65 | NOTCH1_PTPRF | TTTATGTACGTTGCTGCCGCAGCCTTTGTACTTCTCTTCTTCGTTGGGTGCGGCGTCT |
| | nucleic acid | TACTAAGCAAACGCAAAAGAACCCAC |
| 66 | NOTCH1_CSPG4 | TTCATGTACGTTGCTGCCGCAGCCTTTGTATTACTCTTTTTCGTTGGGTGCGGAGTCC |
| | nucleic acid | TTCTCAGCCGGAAACGCAACAAAACCGGCAAACAC |
| 67 | SEM4F_NOTCH2 | GGTGCAGGCTTAGCTGGGTTTTTCCTCGGAATACTCGCCGCCAGCCTTACCCTAATAC |
| | nucleic acid | TTATAGGCAAACGCAAACGAAAACAC |
| 68 | IRAG1_CD2 | TGGCAAGTTATCTGGATGATGGCCGCTGTGATGCTAGTGCTGACAGTCGTGCTGGGCT |
| | nucleic acid | TATACAAGCGGAAAAAGCAGCGCAGCAGAAGG |
| 69 | SIGL9_CD2_nucleic | GTGGTCGGTGGCGCGGGAGCTACCGCCCTCGTGTTCCTGTCCTTTTGCGTGATCTTCG |
| | acid | TTGTAAAAAGGAAGAAGCAGCGGAGCAGACGC |
| 70 | IRAG1_NOTCH1 | TGGCAGGTTATATGGATGATGGCTGCCGTAATGTTAGTTCTAACCGTCGTCCTCGGCC |
| | nucleic acid | TTTACAGCCGGAAACGACGCAGA |
| 71 | NOTCH1_SLAF1 | TTCATGTACGTTGCTGCCGCAGCCTTTGTATTACTCTTTTTCGTTGGGTGCGGAGTCC |
| | nucleic acid | TTCTAAGCCGAAGACGCGGCAAAACAAACCAC |
| 72 | NOTCH1_TMIG3 | TTCATGTACGTAGCTGCCGCAGCCTTCGTTCTTCTCTTTTTTGTTGGGTGCGGCGTCT |
| | nucleic acid | TACTAAGCAAAAGACGCAGATCCCAGCGGAACCGTCGA |
| 73 | NOTCH1_C163A | TTTATGTACGTTGCTGCCGCAGCCTTCGTACTTCTCTTCTTTGTTGGGTGCGGCGTCC |
| | nucleic acid | TATTAAGCAAAAAACGACGCCAGAGACAACGG |
| 74 | PLXC1_EPHA1 | TGGTATTTTCTTATCGTGCTCCCGGTACTGCTAGTGATCGTGATTTTCGCTGCCGTTG |
| | nucleic acid | GCGTGCGAAGCAGAAGGGCACAGCGCCAGAGACAACAGAGGCAGCGGGACCGT |
| 75 | Notchl_ext_Notch1 | TTCATGTACGTGGCGGCGGCCGCCTTTGTGCTTCTGTTCTTCGTGGGCTGCGGGGTGC |
| | nucleic acid | TGCTGTCCCGTAAACGCAGACGTCAACACGGTCAACTGTGGTTTCCAGAAGGTTTTAA |
| | | GGTCTCCGAAGCAAGTAAGAAGAAAAGACGTGAACCACTGGGAGAAGATAGCGTCGGT |
| | | CTGAAACCACTCAAGAATGCC |
| 76 | ALPG/P binder | DIQMTQSPSSLSASVGDRVTITCRASENIYSYVAWYQQKPGKAPKLLIYNAKSLASGV |
| | amino acid | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSS |
| 77 | ALPG/P priming | DIQMTQSPSSLSASVGDRVTITCRASENIYSYVAWYQQKPGKAPKLLIYNAKSLASGV |
| | receptor Notch1-ext- | PSRFSGSGSGTDFTLTISSLQPEDFATYYCQHHYVSPWTFGGGTKLEIKGGGGSGGGG |
| | notch1 TMD/JMD | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSRKRRRQHGQLWFPEGFKVSEASKKKRREPLGEDSVGLKPLKNAMVSKLSQLQTEL |
| | | LAALLESGLSKEALLQALGEPGPYLLAGEGPLDKGESCGGGRGELAELPNGLGETRGS |
| | | EDETDDDGEDFTPPILKELENLSPEEAAHQKAVVETLLQEDPWRVAKMVKSYLQQHNI |
| | | PQREVVDTTGLNQSHLSQHLNKGTPMKTQKRAALYTWYVRKQREVAQQFTHAGQGGLI |
| | | EEPTGDELPTKKGRRNRFKWGPASQQILFQAYERQKNPSKEERETLVEECNRAECIQR |
| | | GVSPSQAQGLGSNLVTEVRVYNWFANRRKEEAFRHKLAMTCRDEFPTMVFPSGQISQA |
| | | SALAPAPPQVLPQAPAPAPAPAMVSALAQAPAPVPVLAPGPPQAVAPPAPKPTQAGEG |
| | | TLSEALLQLQFDDEDLGALLGNSTDPAVFTDLASVDNSEFQQLLNQGIPVAPHTTEPM |
| | | LMEYPEAITRLVTGAQRPPDPAPAPLGAPGLPNGLLSGDEDESSIADMDESALLSQIS |
| | | S |
| 78 | MSLN scFv protein | QVQLQQSGPELEKPGASVKISCKASGYSFTGYTMNWVKQSHGKSLEWIGLITPYNGAS |
| | full sequence | SYNQKFRGKATLTVDKSSSTAYMDLLSLTSEDSAVYFCARGGYDGRGFDYWGQGTTVT |
| | | VSSGGGGSGGGGSGGGGSDIELTQSPAIMSASPGEKVTMTCSASSSVSYMHWYQQKSG |
| | | TSPKRWIYDTSKLASGVPGRFSGSGSGNSYSLTISSVEAEDDATYYCQQWSGYPLTFG |
| | | AGTKLEIK |
| 79 | MSLN CAR protein | QVQLQQSGPELEKPGASVKISCKASGYSFTGYTMNWVKQSHGKSLEWIGLITPYNGAS |
| | full sequence | SYNQKFRGKATLTVDKSSSTAYMDLLSLTSEDSAVYFCARGGYDGRGFDYWGQGTTVT |
| | | VSSGGGGSGGGGSGGGGSDIELTQSPAIMSASPGEKVTMTCSASSSVSYMHWYQQKSG |
| | | TSPKRWIYDTSKLASGVPGRFSGSGSGNSYSLTISSVEAEDDATYYCQQWSGYPLTFG |
| | | AGTKLEIKAAATTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYDALHMQALP |
| | | PR |
| 80 | priming receptor | MVSKLSQLQTELLAALLESGLSKEALLQALGEPGPYLLAGEGPLDKGESCGGGRGELA |
| | HNFla DBD | ELPNGLGETRGSEDETDDDGEDFTPPILKELENLSPEEAAHQKAVVETLLQEDPWRVA |
| | | KMVKSYLQQHNIPQREVVDTTGLNQSHLSQHLNKGTPMKTQKRAALYTWYVRKQREVA |
| | | QQFTHAGQGGLIEEPTGDELPTKKGRRNRFKWGPASQQILFQAYERQKNPSKEERETL |
| | | VEECNRAECIQRGVSPSQAQGLGSNLVTEVRVYNWFANRRKEEAFRHKLAM |
| 81 | priming receptor p65 | DEFPTMVFPSGQISQASALAPAPPQVLPQAPAPAPAPAMVSALAQAPAPVPVLAPGPP |
| | TAD | QAVAPPAPKPTQAGEGTLSEALLQLQFDDEDLGALLGNSTDPAVFTDLASVDNSEFQQ |
| | | LLNQGIPVAPHTTEPMLMEYPEAITRLVTGAQRPPDPAPAPLGAPGLPNGLLSGDEDE |
| | | SSIADMDFSALLSQISS |
| 82 | priming receptor | MVSKLSQLQTELLAALLESGLSKEALLQALGEPGPYLLAGEGPLDKGESCGGGRGELA |
| | HNFla- p65 TAD | ELPNGLGETRGSEDETDDDGEDFTPPILKELENLSPEEAAHQKAVVETLLQEDPWRVA |
| | | KMVKSYLQQHNIPQREVVDTTGLNQSHLSQHLNKGTPMKTQKRAALYTWYVRKQREVA |
| | | QQFTHAGQGGLIEEPTGDELPTKKGRRNRFKWGPASQQILFQAYERQKNPSKEERETL |
| | | VEECNRAECIQRGVSPSQAQGLGSNLVTEVRVYNWFANRRKEEAFRHKLAMTCRDEFP |
| | | TMVFPSGQISQASALAPAPPQVLPQAPAPAPAPAMVSALAQAPAPVPVLAPGPPQAVA |
| | | PPAPKPTQAGEGTLSEALLQLQFDDEDLGALLGNSTDPAVFTDLASVDNSEFQQLLNQ |
| | | GIPVAPHTTEPMLMEYPEAITRLVTGAQRPPDPAPAPLGAPGLPNGLLSGDEDESSIA |
| | | DMDESALLSQISS |
| 83 | priming receptor | TTTPAPRPPTPAPTIASQPLSLRPEAC |
| | CD8 Hinge | |
| 84 | CAR Hinge CD8a | TTTPAPRPPTPAPTIASQPLSLRPEACRPAAGGAVHTRGLDFACD |
| 85 | CAR TMD CD8a | IYIWAPLAGTCGVLLLSLVITLYC |
| 86 | CAR Co-stim 4-1BB | KRGRKKLLYIFKQPFMRPVQTTQEEDGCSCRFPEEEEGGCEL |
| 87 | CAR Activation | RVKFSRSADAPAYQQGQNQLYNELNLGRREEYDVLDKRRGRDPEMGGKPRRKNPQEGL |
| | CD3z | YNELQKDKMAEAYSEIGMKGERRRGKGHDGLYQGLSTATKDTYDALHMQALPPR |
| 88 | APLG/P CDR-H1 | GFSLTSY--- |
| 89 | APLG/P CDR-H1 | GFSLTSYGVS |
| 90 | APLG/P CDR-H1 | -----SYGVS |
| 91 | APLG/P CDR-H1 | ----TSYGVS |
| 92 | APLG/P CDR-H1 | GFSLTSYG-- |
| 93 | APLG/P CDR-H2 | -----WEDGS--------- |
| 94 | APLG/P CDR-H2 | ---VIWEDGSTN------- |
| 95 | APLG/P CDR-H2 | ---VIWEDGSTNYHSALIS |
| 96 | APLG/P CDR-H2 | WIGVIWEDGSTN- |
| 97 | APLG/P CDR-H2 | -IWEDGST----- |
| 98 | APLG/P CDR-H3 | --PHYGSSYVGAMEY |
| 99 | APLG/P CDR-H3 | ARPHYGSSYVGAME- |
| 100 | APLG/P CDR-H3 | ARPHYGSSYVGAMEY |
| 101 | APLG/P CDR-L1 | RASENIYSYVA-- |
| 102 | APLG/P CDR-L1 | ------YSYVAWY |
| 103 | APLG/P CDR-L1 | ---ENIYSY---- |
| 104 | APLG/P CDR-L2 | ----NAKSLAS |
| 105 | APLG/P CDR-L2 | LLIYNAKSLA- |
| — | APLG/P CDR-L2 | ----NA----- |
| 107 | APLG/P CDR-L3 | QHHYVSPWT |
| 108 | APLG/P CDR-L3 | QHHYVSPW- |
| 109 | APLG/P VL | DIQMTQSPSSLSASVGDRVTITCRASENIYSYVAWYQQKPGKAPKLLIYNAKSLASGV |
| | | PSRFSGSGSGTDFTLTISSLQPEDFATYYCQHHYVSPWTFGGGTKLEIK |
| 110 | APLG/P VH | QVQLQESGPGLVKPSETLSLTCTVSGFSLTSYGVSWIRQPAGKGLEWIGVIWEDGSTN |
| | | YHSALISRVTMSVDTSKNQFSLKLSSVTAADTAVYYCARPHYGSSYVGAMEYWGAGTT |
| | | VTVSS |
| 111 | PLXC1_EPHA1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACWYFLIVLPVLLVIVIFAAV |
| | | GVRSRRAQRQRQQRQRDRMVSKLSQLQTELLAALLESGLSKEALLQALGEPGPYLLAG |
| | | EGPLDKGESCGGGRGELAELPNGLGETRGSEDETDDDGEDFTPPILKELENLSPEEAA |
| | | HQKAVVETLLQEDPWRVAKMVKSYLQQHNIPQREVVDTTGLNQSHLSQHLNKGTPMKT |
| | | QKRAALYTWYVRKQREVAQQFTHAGQGGLIEEPTGDELPTKKGRRNRFKWGPASQQIL |
| | | FQAYERQKNPSKEERETLVEECNRAECIQRGVSPSQAQGLGSNLVTEVRVYNWFANRR |
| | | KEEAFRHKLAMTCRDEFPTMVFPSGQISQASALAPAPPQVLPQAPAPAPAPAMVSALA |
| | | QAPAPVPVLAPGPPQAVAPPAPKPTQAGEGTLSEALLQLQFDDEDLGALLGNSTDPAV |
| | | FTDLASVDNSEFQQLLNQGIPVAPHTTEPMLMEYPEAITRLVTGAQRPPDPAPAPLGA |
| | | PGLPNGLLSGDEDFSSIADMDFSALLSQISS |
| 112 | PLXC1_CD2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACWYFLIVLPVLLVIVIFAAV |
| | | GVKRKKQRSRRMVSKLSQLQTELLAALLESGLSKEALLQALGEPGPYLLAGEGPLDKG |
| | | ESCGGGRGELAELPNGLGETRGSEDETDDDGEDFTPPILKELENLSPEEAAHQKAVVE |
| | | TLLQEDPWRVAKMVKSYLQQHNIPQREVVDTTGLNQSHLSQHLNKGTPMKTQKRAALY |
| | | TWYVRKQREVAQQFTHAGQGGLIEEPTGDELPTKKGRRNRFKWGPASQQILFQAYERQ |
| | | KNPSKEERETLVEECNRAECIQRGVSPSQAQGLGSNLVTEVRVYNWFANRRKEEAFRH |
| | | KLAMTCRDEFPTMVFPSGQISQASALAPAPPQVLPQAPAPAPAPAMVSALAQAPAPVP |
| | | VLAPGPPQAVAPPAPKPTQAGEGTLSEALLQLQFDDEDLGALLGNSTDPAVFTDLASV |
| | | DNSEFQQLLNQGIPVAPHTTEPMLMEYPEAITRLVTGAQRPPDPAPAPLGAPGLPNGL |
| | | LSGDEDFSSIADMDFSALLSQISS |
| 113 | NOTCH1_TMIG3 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSKRRRSQRNRRMVSKLSQLQTELLAALLESGLSKEALLQALGEPGPYLLAGEGPLD |
| | | KGESCGGGRGELAELPNGLGETRGSEDETDDDGEDFTPPILKELENLSPEEAAHQKAV |
| | | VETLLQEDPWRVAKMVKSYLQQHNIPQREVVDTTGLNQSHLSQHLNKGTPMKTQKRAA |
| | | LYTWYVRKQREVAQQFTHAGQGGLIEEPTGDELPTKKGRRNRFKWGPASQQILFQAYE |
| | | RQKNPSKEERETLVEECNRAECIQRGVSPSQAQGLGSNLVTEVRVYNWFANRRKEEAF |
| | | RHKLAMTCRDEFPTMVFPSGQISQASALAPAPPQVLPQAPAPAPAPAMVSALAQAPAP |
| | | VPVLAPGPPQAVAPPAPKPTQAGEGTLSEALLQLQFDDEDLGALLGNSTDPAVFTDLA |
| | | SVDNSEFQQLLNQGIPVAPHTTEPMLMEYPEAITRLVTGAQRPPDPAPAPLGAPGLPN |
| | | GLLSGDEDFSSIADMDFSALLSQISS |
| 114 | NOTCH1_PTPRF | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSKRKRTHMVSKLSQLQTELLAALLESGLSKEALLQALGEPGPYLLAGEGPLDKGES |
| | | CGGGRGELAELPNGLGETRGSEDETDDDGEDFTPPILKELENLSPEEAAHQKAVVETL |
| | | LQEDPWRVAKMVKSYLQQHNIPQREVVDTTGLNQSHLSQHLNKGTPMKTQKRAALYTW |
| | | YVRKQREVAQQFTHAGQGGLIEEPTGDELPTKKGRRNRFKWGPASQQILFQAYERQKN |
| | | PSKEERETLVEECNRAECIQRGVSPSQAQGLGSNLVTEVRVYNWFANRRKEEAFRHKL |
| | | AMTCRDEFPTMVFPSGQISQASALAPAPPQVLPQAPAPAPAPAMVSALAQAPAPVPVL |
| | | APGPPQAVAPPAPKPTQAGEGTLSEALLQLQFDDEDLGALLGNSTDPAVFTDLASVDN |
| | | SEFQQLLNQGIPVAPHTTEPMLMEYPEAITRLVTGAQRPPDPAPAPLGAPGLPNGLLS |
| | | GDEDFSSIADMDFSALLSQISS |
| 115 | ECSCR_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACFGVISFIVILVVVVIILVG |
| | | VVSRKRRRMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDFSSIADMDFSALLSQISS |
| 116 | MMEL1_NOTCH2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACGLLLLLLLVTAALVALGVL |
| | | YADKRKRKHMVSKLSQLQTELLAALLESGLSKEALLQALGEPGPYLLAGEGPLDKGES |
| | | CGGGRGELAELPNGLGETRGSEDETDDDGEDFTPPILKELENLSPEEAAHQKAVVETL |
| | | LQEDPWRVAKMVKSYLQQHNIPQREVVDTTGLNQSHLSQHLNKGTPMKTQKRAALYTW |
| | | YVRKQREVAQQFTHAGQGGLIEEPTGDELPTKKGRRNRFKWGPASQQILFQAYERQKN |
| | | PSKEERETLVEECNRAECIQRGVSPSQAQGLGSNLVTEVRVYNWFANRRKEEAFRHKL |
| | | AMTCRDEFPTMVFPSGQISQASALAPAPPQVLPQAPAPAPAPAMVSALAQAPAPVPVL |
| | | APGPPQAVAPPAPKPTQAGEGTLSEALLQLQFDDEDLGALLGNSTDPAVFTDLASVDN |
| | | SEFQQLLNQGIPVAPHTTEPMLMEYPEAITRLVTGAQRPPDPAPAPLGAPGLPNGLLS |
| | | GDEDESSIADMDFSALLSQISS |
| 117 | IRAG1_C163A | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACWQVIWMMAAVMLVLTVVLG |
| | | LYKKRRQRQRMVSKLSQLQTELLAALLESGLSKEALLQALGEPGPYLLAGEGPLDKGE |
| | | SCGGGRGELAELPNGLGETRGSEDETDDDGEDFTPPILKELENLSPEEAAHQKAVVET |
| | | LLQEDPWRVAKMVKSYLQQHNIPQREVVDTTGLNQSHLSQHLNKGTPMKTQKRAALYT |
| | | WYVRKQREVAQQFTHAGQGGLIEEPTGDELPTKKGRRNRFKWGPASQQILFQAYERQK |
| | | NPSKEERETLVEECNRAECIQRGVSPSQAQGLGSNLVTEVRVYNWFANRRKEEAFRHK |
| | | LAMTCRDEFPTMVFPSGQISQASALAPAPPQVLPQAPAPAPAPAMVSALAQAPAPVPV |
| | | LAPGPPQAVAPPAPKPTQAGEGTLSEALLQLQFDDEDLGALLGNSTDPAVFTDLASVD |
| | | NSEFQQLLNQGIPVAPHTTEPMLMEYPEAITRLVTGAQRPPDPAPAPLGAPGLPNGLL |
| | | SGDEDFSSIADMDFSALLSQISS |
| 118 | IRAG1_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACWQVIWMMAAVMLVLTVVLG |
| | | LYSRKRRRMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDESSIADMDFSALLSQISS |
| 119 | NOTCH1_CSPG4 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSRKRNKTGKHMVSKLSQLQTELLAALLESGLSKEALLQALGEPGPYLLAGEGPLDK |
| | | GESCGGGRGELAELPNGLGETRGSEDETDDDGEDFTPPILKELENLSPEEAAHQKAVV |
| | | ETLLQEDPWRVAKMVKSYLQQHNIPQREVVDTTGLNQSHLSQHLNKGTPMKTQKRAAL |
| | | YTWYVRKQREVAQQFTHAGQGGLIEEPTGDELPTKKGRRNRFKWGPASQQILFQAYER |
| | | QKNPSKEERETLVEECNRAECIQRGVSPSQAQGLGSNLVTEVRVYNWFANRRKEEAFR |
| | | HKLAMTCRDEFPTMVFPSGQISQASALAPAPPQVLPQAPAPAPAPAMVSALAQAPAPV |
| | | PVLAPGPPQAVAPPAPKPTQAGEGTLSEALLQLQFDDEDLGALLGNSTDPAVFTDLAS |
| | | VDNSEFQQLLNQGIPVAPHTTEPMLMEYPEAITRLVTGAQRPPDPAPAPLGAPGLPNG |
| | | LLSGDEDFSSIADMDFSALLSQISS |
| 120 | NOTCH1_C163A | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACFMYVAAAAFVLLFFVGCGV |
| | | LLSKKRRQRQRMVSKLSQLQTELLAALLESGLSKEALLQALGEPGPYLLAGEGPLDKG |
| | | ESCGGGRGELAELPNGLGETRGSEDETDDDGEDFTPPILKELENLSPEEAAHQKAVVE |
| | | TLLQEDPWRVAKMVKSYLQQHNIPQREVVDTTGLNQSHLSQHLNKGTPMKTQKRAALY |
| | | TWYVRKQREVAQQFTHAGQGGLIEEPTGDELPTKKGRRNRFKWGPASQQILFQAYERQ |
| | | KNPSKEERETLVEECNRAECIQRGVSPSQAQGLGSNLVTEVRVYNWFANRRKEEAFRH |
| | | KLAMTCRDEFPTMVFPSGQISQASALAPAPPQVLPQAPAPAPAPAMVSALAQAPAPVP |
| | | VLAPGPPQAVAPPAPKPTQAGEGTLSEALLQLQFDDEDLGALLGNSTDPAVFTDLASV |
| | | DNSEFQQLLNQGIPVAPHTTEPMLMEYPEAITRLVTGAQRPPDPAPAPLGAPGLPNGL |
| | | LSGDEDESSIADMDFSALLSQISS |
| 121 | DCBD1_NOTCH2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACAIPLVLLVVLVFAGMGIFA |
| | | AFKRKRKHMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDFSSIADMDFSALLSQISS |
| 122 | MCP_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACVWVIAVIVIAIVVGVAVIC |
| | | VVPYSRKRRRMVSKLSQLQTELLAALLESGLSKEALLQALGEPGPYLLAGEGPLDKGE |
| | | SCGGGRGELAELPNGLGETRGSEDETDDDGEDFTPPILKELENLSPEEAAHQKAVVET |
| | | LLQEDPWRVAKMVKSYLQQHNIPQREVVDTTGLNQSHLSQHLNKGTPMKTQKRAALYT |
| | | WYVRKQREVAQQFTHAGQGGLIEEPTGDELPTKKGRRNRFKWGPASQQILFQAYERQK |
| | | NPSKEERETLVEECNRAECIQRGVSPSQAQGLGSNLVTEVRVYNWFANRRKEEAFRHK |
| | | LAMTCRDEFPTMVFPSGQISQASALAPAPPQVLPQAPAPAPAPAMVSALAQAPAPVPV |
| | | LAPGPPQAVAPPAPKPTQAGEGTLSEALLQLQFDDEDLGALLGNSTDPAVFTDLASVD |
| | | NSEFQQLLNQGIPVAPHTTEPMLMEYPEAITRLVTGAQRPPDPAPAPLGAPGLPNGLL |
| | | SGDEDFSSIADMDFSALLSQISS |
| 123 | HMR1_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACKAVSGSIVLVIVLAGVGVL |
| | | VWSRKRRRMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDFSSIADMDFSALLSQISS |
| 124 | IRPL1_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACVELAGGLGAILLLLVCLVT |
| | | IYSRKRRRMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDFSSIADMDFSALLSQISS |
| 125 | DISP1_NOTCH2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACVVVLGMCTMFIVVCALVGV |
| | | LVKRKRKHMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDESSIADMDFSALLSQISS |
| 126 | SEM4F_NOTCH2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACGAGLAGFFLGILAASLTLI |
| | | LIGKRKRKHMVSKLSQLQTELLAALLESGLSKEALLQALGEPGPYLLAGEGPLDKGES |
| | | CGGGRGELAELPNGLGETRGSEDETDDDGEDFTPPILKELENLSPEEAAHQKAVVETL |
| | | LQEDPWRVAKMVKSYLQQHNIPQREVVDTTGLNQSHLSQHLNKGTPMKTQKRAALYTW |
| | | YVRKQREVAQQFTHAGQGGLIEEPTGDELPTKKGRRNRFKWGPASQQILFQAYERQKN |
| | | PSKEERETLVEECNRAECIQRGVSPSQAQGLGSNLVTEVRVYNWFANRRKEEAFRHKL |
| | | AMTCRDEFPTMVFPSGQISQASALAPAPPQVLPQAPAPAPAPAMVSALAQAPAPVPVL |
| | | APGPPQAVAPPAPKPTQAGEGTLSEALLQLQFDDEDLGALLGNSTDPAVFTDLASVDN |
| | | SEFQQLLNQGIPVAPHTTEPMLMEYPEAITRLVTGAQRPPDPAPAPLGAPGLPNGLLS |
| | | GDEDFSSIADMDFSALLSQISS |
| 127 | APP_DAG1 PrimeR | DIQMTQSPSSLSASVGDRVTITCRASENIYSYVAWYQQKPGKAPKLLIYNAKSLASGV |
| | | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACGAIIGLMVGGVVIATVIVI |
| | | TLVMLRKKRKGKMVSKLSQLQTELLAALLESGLSKEALLQALGEPGPYLLAGEGPLDK |
| | | GESCGGGRGELAELPNGLGETRGSEDETDDDGEDFTPPILKELENLSPEEAAHQKAVV |
| | | ETLLQEDPWRVAKMVKSYLQQHNIPQREVVDTTGLNQSHLSQHLNKGTPMKTQKRAAL |
| | | YTWYVRKQREVAQQFTHAGQGGLIEEPTGDELPTKKGRRNRFKWGPASQQILFQAYER |
| | | QKNPSKEERETLVEECNRAECIQRGVSPSQAQGLGSNLVTEVRVYNWFANRRKEEAFR |
| | | HKLAMTCRDEFPTMVFPSGQISQASALAPAPPQVLPQAPAPAPAPAMVSALAQAPAPV |
| | | PVLAPGPPQAVAPPAPKPTQAGEGTLSEALLQLQFDDEDLGALLGNSTDPAVFTDLAS |
| | | VDNSEFQQLLNQGIPVAPHTTEPMLMEYPEAITRLVTGAQRPPDPAPAPLGAPGLPNG |
| | | LLSGDEDFSSIADMDFSALLSQISS |
| 128 | ZP1_NOTCH1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACLLWAVLLLPAVALVLGFGV |
| | | FVSRKRRRMVSKLSQLQTELLAALLESGLSKEALLQALGEPGPYLLAGEGPLDKGESC |
| | | GGGRGELAELPNGLGETRGSEDETDDDGEDFTPPILKELENLSPEEAAHQKAVVETLL |
| | | QEDPWRVAKMVKSYLQQHNIPQREVVDTTGLNQSHLSQHLNKGTPMKTQKRAALYTWY |
| | | VRKQREVAQQFTHAGQGGLIEEPTGDELPTKKGRRNRFKWGPASQQILFQAYERQKNP |
| | | SKEERETLVEECNRAECIQRGVSPSQAQGLGSNLVTEVRVYNWFANRRKEEAFRHKLA |
| | | MTCRDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNS |
| | | EFQQLLNQGIPVAPHTTEPMLMEYPEAITRLVTGAQRPPDPAPAPLGAPGLPNGLLSG |
| | | DEDFSSIADMDFSALLSQISS |
| 129 | SIGL9_C163A | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACVVGGAGATALVFLSFCVIF |
| | | VVKKRRQRQRMVSKLSQLQTELLAALLESGLSKEALLQALGEPGPYLLAGEGPLDKGE |
| | | SCGGGRGELAELPNGLGETRGSEDETDDDGEDFTPPILKELENLSPEEAAHQKAVVET |
| | | LLQEDPWRVAKMVKSYLQQHNIPQREVVDTTGLNQSHLSQHLNKGTPMKTQKRAALYT |
| | | WYVRKQREVAQQFTHAGQGGLIEEPTGDELPTKKGRRNRFKWGPASQQILFQAYERQK |
| | | NPSKEERETLVEECNRAECIQRGVSPSQAQGLGSNLVTEVRVYNWFANRRKEEAFRHK |
| | | LAMTCRDEFPTMVFPSGQISQASALAPAPPQVLPQAPAPAPAPAMVSALAQAPAPVPV |
| | | LAPGPPQAVAPPAPKPTQAGEGTLSEALLQLQFDDEDLGALLGNSTDPAVFTDLASVD |
| | | NSEFQQLLNQGIPVAPHTTEPMLMEYPEAITRLVTGAQRPPDPAPAPLGAPGLPNGLL |
| | | SGDEDFSSIADMDFSALLSQISS |
| 130 | IRAG1_CD2 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACWQVIWMMAAVMLVLTVVLG |
| | | LYKRKKQRSRRMVSKLSQLQTELLAALLESGLSKEALLQALGEPGPYLLAGEGPLDKG |
| | | ESCGGGRGELAELPNGLGETRGSEDETDDDGEDFTPPILKELENLSPEEAAHQKAVVE |
| | | TLLQEDPWRVAKMVKSYLQQHNIPQREVVDTTGLNQSHLSQHLNKGTPMKTQKRAALY |
| | | TWYVRKQREVAQQFTHAGQGGLIEEPTGDELPTKKGRRNRFKWGPASQQILFQAYERQ |
| | | KNPSKEERETLVEECNRAECIQRGVSPSQAQGLGSNLVTEVRVYNWFANRRKEEAFRH |
| | | KLAMTCRDEFPTMVFPSGQISQASALAPAPPQVLPQAPAPAPAPAMVSALAQAPAPVP |
| | | VLAPGPPQAVAPPAPKPTQAGEGTLSEALLQLQFDDEDLGALLGNSTDPAVFTDLASV |
| | | DNSEFQQLLNQGIPVAPHTTEPMLMEYPEAITRLVTGAQRPPDPAPAPLGAPGLPNGL |
| | | LSGDEDFSSIADMDFSALLSQISS |
| 131 | Notch1_ext_Notch1 | DIQMTQSPSSLSASVGDRVTITCRASENIYSYVAWYQQKPGKAPKLLIYNAKSLASGV |
| | PrimeR | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSRKRRRQHGQLWFPEGFKVSEASKKKRREPLGEDSVGLKPLKNAMVSKLSQLQTEL |
| | | LAALLESGLSKEALLQALGEPGPYLLAGEGPLDKGESCGGGRGELAELPNGLGETRGS |
| | | EDETDDDGEDFTPPILKELENLSPEEAAHQKAVVETLLQEDPWRVAKMVKSYLQQHNI |
| | | PQREVVDTTGLNQSHLSQHLNKGTPMKTQKRAALYTWYVRKQREVAQQFTHAGQGGLI |
| | | EEPTGDELPTKKGRRNRFKWGPASQQILFQAYERQKNPSKEERETLVEECNRAECIQR |
| | | GVSPSQAQGLGSNLVTEVRVYNWFANRRKEEAFRHKLAMTCRDEFPTMVFPSGQISQA |
| | | SALAPAPPQVLPQAPAPAPAPAMVSALAQAPAPVPVLAPGPPQAVAPPAPKPTQAGEG |
| | | TLSEALLQLQFDDEDLGALLGNSTDPAVFTDLASVDNSEFQQLLNQGIPVAPHTTEPM |
| | | LMEYPEAITRLVTGAQRPPDPAPAPLGAPGLPNGLLSGDEDESSIADMDESALLSQIS |
| | | S |
| 132 | Notch1_stop PrimeR | DIQMTQSPSSLSASVGDRVTITCRASENIYSYVAWYQQKPGKAPKLLIYNAKSLASGV |
| | | PSRFSGSGSGTDFTLTISSLQPEDFATYYCQHHYVSPWTFGGGTKLEIKGGGGSGGGG |
| | | SGGGGSQVQLQESGPGLVKPSETLSLTCTVSGFSLTSYGVSWIRQPAGKGLEWIGVIW |
| | | EDGSTNYHSALISRVTMSVDTSKNQFSLKLSSVTAADTAVYYCARPHYGSSYVGAMEY |
| | | WGAGTTVTVSSATTTPAPRPPTPAPTIASQPLSLRPEACEMYVAAAAFVLLFFVGCGV |
| | | LLSRKRRRQHGQLWFPEGFKVSEASKKKRREPLGEDSVGLKPLKNAMVSKLSQLQTEL |
| | | LAALLESGLSKEALLQALGEPGPYLLAGEGPLDKGESCGGGRGELAELPNGLGETRGS |
| | | EDETDDDGEDFTPPILKELENLSPEEAAHQKAVVETLLQEDPWRVAKMVKSYLQQHNI |
| | | PQREVVDTTGLNQSHLSQHLNKGTPMKTQKRAALYTWYVRKQREVAQQFTHAGQGGLI |
| | | EEPTGDELPTKKGRRNRFKWGPASQQILFQAYERQKNPSKEERETLVEECNRAECIQR |
| | | GVSPSQAQGLGSNLVTEVRVYNWFANRRKEEAFRHKLAMTCRDEFPTMVFPSGQISQA |
| | | SALAPAPPQVLPQAPAPAPAPAMVSALAQAPAPVPVLAPGPPQAVAPPAPKPTQAGEG |
| | | TLSEALLQLQFDDEDLGALLGNSTDPAVFTDLASVDNSEFQQLLNQGIPVAPHTTEPM |
| | | LMEYPEAITRLVTGAQRPPDPAPAPLGAPGLPNGLLSGDEDESSIADMDESALLSQIS |
| | | S |
| 133 | MSLN CDR H1 | GYTMN |
| 134 | MSLN CDR H2 | LITPYNGASSYNQKFRG |
| 135 | MSLN CDR H3 | GGYDGRGFDY |
| 136 | MSLN VH | QVQLQQSGPELEKPGASVKISCKASGYSFTGYTMNWVKQSHGKSLEWIGLITPYNGAS |
| | | SYNQKFRGKATLTVDKSSSTAYMDLLSLTSEDSAVYFCARGGYDGRGFDYWGQGTTVT |
| | | VSS |
| 137 | MSLN CDR L1 | SASSSVSYMH |
| 138 | MSLN CDR L2 | DTSKLAS |
| 139 | MSLN CDR L3 | QQWSGYPLT |
| 140 | MSLN VL | DIELTQSPAIMSASPGEKVTMTCSASSSVSYMHWYQQKSGTSPKRWIYDTSKLASGVP |
| | | GRFSGSGSGNSYSLTISSVEAEDDATYYCQQWSGYPLTFGAGTKLEIK |
| 281 | PLXC1_EPHA1 | TTTPAPRPPTPAPTIASQPLSLRPEACWYFLIVLPVLLVIVIFAAVGVRSRRAQRQRQ |
| | PrimeR | QRQRDRMVSKLSQLQTELLAALLESGLSKEALLQALGEPGPYLLAGEGPLDKGESCGG |
| | | GRGELAELPNGLGETRGSEDETDDDGEDFTPPILKELENLSPEEAAHQKAVVETLLQE |
| | | DPWRVAKMVKSYLQQHNIPQREVVDTTGLNQSHLSQHLNKGTPMKTQKRAALYTWYVR |
| | | KQREVAQQFTHAGQGGLIEEPTGDELPTKKGRRNRFKWGPASQQILFQAYERQKNPSK |
| | | EERETLVEECNRAECIQRGVSPSQAQGLGSNLVTEVRVYNWFANRRKEEAFRHKLAMT |
| | | CRDEFPTMVFPSGQISQASALAPAPPQVLPQAPAPAPAPAMVSALAQAPAPVPVLAPG |
| | | PPQAVAPPAPKPTQAGEGTLSEALLQLQFDDEDLGALLGNSTDPAVFTDLASVDNSEF |
| | | QQLLNQGIPVAPHTTEPMLMEYPEAITRLVTGAQRPPDPAPAPLGAPGLPNGLLSGDE |
| | | DFSSIADMDFSALLSQISS |
| 282 | PLXC1_CD2 | TTTPAPRPPTPAPTIASQPLSLRPEACWYFLIVLPVLLVIVIFAAVGVKRKKQRSRRM |
| | PrimeR | VSKLSQLQTELLAALLESGLSKEALLQALGEPGPYLLAGEGPLDKGESCGGGRGELAE |
| | | LPNGLGETRGSEDETDDDGEDFTPPILKELENLSPEEAAHQKAVVETLLQEDPWRVAK |
| | | MVKSYLQQHNIPQREVVDTTGLNQSHLSQHLNKGTPMKTQKRAALYTWYVRKQREVAQ |
| | | QFTHAGQGGLIEEPTGDELPTKKGRRNRFKWGPASQQILFQAYERQKNPSKEERETLV |
| | | EECNRAECIQRGVSPSQAQGLGSNLVTEVRVYNWFANRRKEEAFRHKLAMTCRDEFPT |
| | | MVFPSGQISQASALAPAPPQVLPQAPAPAPAPAMVSALAQAPAPVPVLAPGPPQAVAP |
| | | PAPKPTQAGEGTLSEALLQLQFDDEDLGALLGNSTDPAVFTDLASVDNSEFQQLLNQG |
| | | IPVAPHTTEPMLMEYPEAITRLVTGAQRPPDPAPAPLGAPGLPNGLLSGDEDFSSIAD |
| | | MDFSALLSQISS |
| 283 | NOTCH1_TMIG3 | TTTPAPRPPTPAPTIASQPLSLRPEACEMYVAAAAFVLLFFVGCGVLLSKRRRSQRNR |
| | PrimeR | RMVSKLSQLQTELLAALLESGLSKEALLQALGEPGPYLLAGEGPLDKGESCGGGRGEL |
| | | AELPNGLGETRGSEDETDDDGEDFTPPILKELENLSPEEAAHQKAVVETLLQEDPWRV |
| | | AKMVKSYLQQHNIPQREVVDTTGLNQSHLSQHLNKGTPMKTQKRAALYTWYVRKQREV |
| | | AQQFTHAGQGGLIEEPTGDELPTKKGRRNRFKWGPASQQILFQAYERQKNPSKEERET |
| | | LVEECNRAECIQRGVSPSQAQGLGSNLVTEVRVYNWFANRRKEEAFRHKLAMTCRDEF |
| | | PTMVFPSGQISQASALAPAPPQVLPQAPAPAPAPAMVSALAQAPAPVPVLAPGPPQAV |
| | | APPAPKPTQAGEGTLSEALLQLQFDDEDLGALLGNSTDPAVFTDLASVDNSEFQQLLN |
| | | QGIPVAPHTTEPMLMEYPEAITRLVTGAQRPPDPAPAPLGAPGLPNGLLSGDEDESSI |
| | | ADMDFSALLSQISS |
| 284 | NOTCH1_PTPRF | TTTPAPRPPTPAPTIASQPLSLRPEACEMYVAAAAFVLLFFVGCGVLLSKRKRTHMVS |
| | PrimeR | KLSQLQTELLAALLESGLSKEALLQALGEPGPYLLAGEGPLDKGESCGGGRGELAELP |
| | | NGLGETRGSEDETDDDGEDFTPPILKELENLSPEEAAHQKAVVETLLQEDPWRVAKMV |
| | | KSYLQQHNIPQREVVDTTGLNQSHLSQHLNKGTPMKTQKRAALYTWYVRKQREVAQQF |
| | | THAGQGGLIEEPTGDELPTKKGRRNRFKWGPASQQILFQAYERQKNPSKEERETLVEE |
| | | CNRAECIQRGVSPSQAQGLGSNLVTEVRVYNWFANRRKEEAFRHKLAMTCRDEFPTMV |
| | | FPSGQISQASALAPAPPQVLPQAPAPAPAPAMVSALAQAPAPVPVLAPGPPQAVAPPA |
| | | PKPTQAGEGTLSEALLQLQFDDEDLGALLGNSTDPAVFTDLASVDNSEFQQLLNQGIP |
| | | VAPHTTEPMLMEYPEAITRLVTGAQRPPDPAPAPLGAPGLPNGLLSGDEDFSSIADMD |
| | | FSALLSQISS |
| 285 | ECSCR_NOTCH1 | TTTPAPRPPTPAPTIASQPLSLRPEACFGVISFIVILVVVVIILVGVVSRKRRRMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDFSSIADMDF |
| | | SALLSQISS |
| 286 | MMEL1_NOTCH2 | TTTPAPRPPTPAPTIASQPLSLRPEACGLLLLLLLVTAALVALGVLYADKRKRKHMVS |
| | PrimeR | KLSQLQTELLAALLESGLSKEALLQALGEPGPYLLAGEGPLDKGESCGGGRGELAELP |
| | | NGLGETRGSEDETDDDGEDFTPPILKELENLSPEEAAHQKAVVETLLQEDPWRVAKMV |
| | | KSYLQQHNIPQREVVDTTGLNQSHLSQHLNKGTPMKTQKRAALYTWYVRKQREVAQQF |
| | | THAGQGGLIEEPTGDELPTKKGRRNRFKWGPASQQILFQAYERQKNPSKEERETLVEE |
| | | CNRAECIQRGVSPSQAQGLGSNLVTEVRVYNWFANRRKEEAFRHKLAMTCRDEFPTMV |
| | | FPSGQISQASALAPAPPQVLPQAPAPAPAPAMVSALAQAPAPVPVLAPGPPQAVAPPA |
| | | PKPTQAGEGTLSEALLQLQFDDEDLGALLGNSTDPAVFTDLASVDNSEFQQLLNQGIP |
| | | VAPHTTEPMLMEYPEAITRLVTGAQRPPDPAPAPLGAPGLPNGLLSGDEDFSSIADMD |
| | | FSALLSQISS |
| 287 | IRAG1_C163A | TTTPAPRPPTPAPTIASQPLSLRPEACWQVIWMMAAVMLVLTVVLGLYKKRRQRQRMV |
| | PrimeR | SKLSQLQTELLAALLESGLSKEALLQALGEPGPYLLAGEGPLDKGESCGGGRGELAEL |
| | | PNGLGETRGSEDETDDDGEDFTPPILKELENLSPEEAAHQKAVVETLLQEDPWRVAKM |
| | | VKSYLQQHNIPQREVVDTTGLNQSHLSQHLNKGTPMKTQKRAALYTWYVRKQREVAQQ |
| | | FTHAGQGGLIEEPTGDELPTKKGRRNRFKWGPASQQILFQAYERQKNPSKEERETLVE |
| | | ECNRAECIQRGVSPSQAQGLGSNLVTEVRVYNWFANRRKEEAFRHKLAMTCRDEFPTM |
| | | VFPSGQISQASALAPAPPQVLPQAPAPAPAPAMVSALAQAPAPVPVLAPGPPQAVAPP |
| | | APKPTQAGEGTLSEALLQLQFDDEDLGALLGNSTDPAVFTDLASVDNSEFQQLLNQGI |
| | | PVAPHTTEPMLMEYPEAITRLVTGAQRPPDPAPAPLGAPGLPNGLLSGDEDFSSIADM |
| | | DFSALLSQISS |
| 288 | IRAG1_NOTCH1 | TTTPAPRPPTPAPTIASQPLSLRPEACWQVIWMMAAVMLVLTVVLGLYSRKRRRMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDFSSIADMDF |
| | | SALLSQISS |
| 289 | NOTCH1_CSPG4 | TTTPAPRPPTPAPTIASQPLSLRPEACEMYVAAAAFVLLFFVGCGVLLSRKRNKTGKH |
| | PrimeR | MVSKLSQLQTELLAALLESGLSKEALLQALGEPGPYLLAGEGPLDKGESCGGGRGELA |
| | | ELPNGLGETRGSEDETDDDGEDFTPPILKELENLSPEEAAHQKAVVETLLQEDPWRVA |
| | | KMVKSYLQQHNIPQREVVDTTGLNQSHLSQHLNKGTPMKTQKRAALYTWYVRKQREVA |
| | | QQFTHAGQGGLIEEPTGDELPTKKGRRNRFKWGPASQQILFQAYERQKNPSKEERETL |
| | | VEECNRAECIQRGVSPSQAQGLGSNLVTEVRVYNWFANRRKEEAFRHKLAMTCRDEFP |
| | | TMVFPSGQISQASALAPAPPQVLPQAPAPAPAPAMVSALAQAPAPVPVLAPGPPQAVA |
| | | PPAPKPTQAGEGTLSEALLQLQFDDEDLGALLGNSTDPAVFTDLASVDNSEFQQLLNQ |
| | | GIPVAPHTTEPMLMEYPEAITRLVTGAQRPPDPAPAPLGAPGLPNGLLSGDEDESSIA |
| | | DMDFSALLSQISS |
| 290 | NOTCH1_C163A | TTTPAPRPPTPAPTIASQPLSLRPEACEMYVAAAAFVLLFFVGCGVLLSKKRRQRQRM |
| | PrimeR | VSKLSQLQTELLAALLESGLSKEALLQALGEPGPYLLAGEGPLDKGESCGGGRGELAE |
| | | LPNGLGETRGSEDETDDDGEDFTPPILKELENLSPEEAAHQKAVVETLLQEDPWRVAK |
| | | MVKSYLQQHNIPQREVVDTTGLNQSHLSQHLNKGTPMKTQKRAALYTWYVRKQREVAQ |
| | | QFTHAGQGGLIEEPTGDELPTKKGRRNRFKWGPASQQILFQAYERQKNPSKEERETLV |
| | | EECNRAECIQRGVSPSQAQGLGSNLVTEVRVYNWFANRRKEEAFRHKLAMTCRDEFPT |
| | | MVFPSGQISQASALAPAPPQVLPQAPAPAPAPAMVSALAQAPAPVPVLAPGPPQAVAP |
| | | PAPKPTQAGEGTLSEALLQLQFDDEDLGALLGNSTDPAVFTDLASVDNSEFQQLLNQG |
| | | IPVAPHTTEPMLMEYPEAITRLVTGAQRPPDPAPAPLGAPGLPNGLLSGDEDESSIAD |
| | | MDFSALLSQISS |
| 291 | DCBD1_NOTCH2 | TTTPAPRPPTPAPTIASQPLSLRPEACAIPLVLLVVLVFAGMGIFAAFKRKRKHMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDFSSIADMDF |
| | | SALLSQISS |
| 292 | MCP_NOTCH1 | TTTPAPRPPTPAPTIASQPLSLRPEACVWVIAVIVIAIVVGVAVICVVPYSRKRRRMV |
| | PrimeR | SKLSQLQTELLAALLESGLSKEALLQALGEPGPYLLAGEGPLDKGESCGGGRGELAEL |
| | | PNGLGETRGSEDETDDDGEDFTPPILKELENLSPEEAAHQKAVVETLLQEDPWRVAKM |
| | | VKSYLQQHNIPQREVVDTTGLNQSHLSQHLNKGTPMKTQKRAALYTWYVRKQREVAQQ |
| | | FTHAGQGGLIEEPTGDELPTKKGRRNRFKWGPASQQILFQAYERQKNPSKEERETLVE |
| | | ECNRAECIQRGVSPSQAQGLGSNLVTEVRVYNWFANRRKEEAFRHKLAMTCRDEFPTM |
| | | VFPSGQISQASALAPAPPQVLPQAPAPAPAPAMVSALAQAPAPVPVLAPGPPQAVAPP |
| | | APKPTQAGEGTLSEALLQLQFDDEDLGALLGNSTDPAVFTDLASVDNSEFQQLLNQGI |
| | | PVAPHTTEPMLMEYPEAITRLVTGAQRPPDPAPAPLGAPGLPNGLLSGDEDFSSIADM |
| | | DESALLSQISS |
| 293 | HMR1_NOTCH1 | TTTPAPRPPTPAPTIASQPLSLRPEACKAVSGSIVLVIVLAGVGVLVWSRKRRRMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDESSIADMDF |
| | | SALLSQISS |
| 294 | IRPL1_NOTCH1 | TTTPAPRPPTPAPTIASQPLSLRPEACVELAGGLGAILLLLVCLVTIYSRKRRRMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVE |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDESSIADMDF |
| | | SALLSQISS |
| 295 | DISP1_NOTCH2 | TTTPAPRPPTPAPTIASQPLSLRPEACVVVLGMCTMFIVVCALVGVLVKRKRKHMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDESSIADMDF |
| | | SALLSQISS |
| 296 | SEM4F_NOTCH2 | TTTPAPRPPTPAPTIASQPLSLRPEACGAGLAGFFLGILAASLTLILIGKRKRKHMVS |
| | PrimeR | KLSQLQTELLAALLESGLSKEALLQALGEPGPYLLAGEGPLDKGESCGGGRGELAELP |
| | | NGLGETRGSEDETDDDGEDFTPPILKELENLSPEEAAHQKAVVETLLQEDPWRVAKMV |
| | | KSYLQQHNIPQREVVDTTGLNQSHLSQHLNKGTPMKTQKRAALYTWYVRKQREVAQQF |
| | | THAGQGGLIEEPTGDELPTKKGRRNRFKWGPASQQILFQAYERQKNPSKEERETLVEE |
| | | CNRAECIQRGVSPSQAQGLGSNLVTEVRVYNWFANRRKEEAFRHKLAMTCRDEFPTMV |
| | | FPSGQISQASALAPAPPQVLPQAPAPAPAPAMVSALAQAPAPVPVLAPGPPQAVAPPA |
| | | PKPTQAGEGTLSEALLQLQFDDEDLGALLGNSTDPAVFTDLASVDNSEFQQLLNQGIP |
| | | VAPHTTEPMLMEYPEAITRLVTGAQRPPDPAPAPLGAPGLPNGLLSGDEDESSIADMD |
| | | FSALLSQISS |
| 297 | APP_DAG1_ PrimeR | TTTPAPRPPTPAPTIASQPLSLRPEACGAIIGLMVGGVVIATVIVITLVMLRKKRKGK |
| | | MVSKLSQLQTELLAALLESGLSKEALLQALGEPGPYLLAGEGPLDKGESCGGGRGELA |
| | | ELPNGLGETRGSEDETDDDGEDFTPPILKELENLSPEEAAHQKAVVETLLQEDPWRVA |
| | | KMVKSYLQQHNIPQREVVDTTGLNQSHLSQHLNKGTPMKTQKRAALYTWYVRKQREVA |
| | | QQFTHAGQGGLIEEPTGDELPTKKGRRNRFKWGPASQQILFQAYERQKNPSKEERETL |
| | | VEECNRAECIQRGVSPSQAQGLGSNLVTEVRVYNWFANRRKEEAFRHKLAMTCRDEFP |
| | | TMVFPSGQISQASALAPAPPQVLPQAPAPAPAPAMVSALAQAPAPVPVLAPGPPQAVA |
| | | PPAPKPTQAGEGTLSEALLQLQFDDEDLGALLGNSTDPAVFTDLASVDNSEFQQLLNQ |
| | | GIPVAPHTTEPMLMEYPEAITRLVTGAQRPPDPAPAPLGAPGLPNGLLSGDEDFSSIA |
| | | DMDFSALLSQISS |
| 298 | ZP1_NOTCH1 | TTTPAPRPPTPAPTIASQPLSLRPEACLLWAVLLLPAVALVLGFGVFVSRKRRRMVSK |
| | PrimeR | LSQLQTELLAALLESGLSKEALLQALGEPGPYLLAGEGPLDKGESCGGGRGELAELPN |
| | | GLGETRGSEDETDDDGEDFTPPILKELENLSPEEAAHQKAVVETLLQEDPWRVAKMVK |
| | | SYLQQHNIPQREVVDTTGLNQSHLSQHLNKGTPMKTQKRAALYTWYVRKQREVAQQFT |
| | | HAGQGGLIEEPTGDELPTKKGRRNRFKWGPASQQILFQAYERQKNPSKEERETLVEEC |
| | | NRAECIQRGVSPSQAQGLGSNLVTEVRVYNWFANRRKEEAFRHKLAMTCRDEFPTMVF |
| | | PSGQISQASALAPAPPQVLPQAPAPAPAPAMVSALAQAPAPVPVLAPGPPQAVAPPAP |
| | | KPTQAGEGTLSEALLQLQFDDEDLGALLGNSTDPAVFTDLASVDNSEFQQLLNQGIPV |
| | | APHTTEPMLMEYPEAITRLVTGAQRPPDPAPAPLGAPGLPNGLLSGDEDESSIADMDF |
| | | SALLSQISS |
| 299 | SIGL9_C163A | TTTPAPRPPTPAPTIASQPLSLRPEACVVGGAGATALVELSFCVIFVVKKRRQRQRMV |
| | PrimeR | SKLSQLQTELLAALLESGLSKEALLQALGEPGPYLLAGEGPLDKGESCGGGRGELAEL |
| | | PNGLGETRGSEDETDDDGEDFTPPILKELENLSPEEAAHQKAVVETLLQEDPWRVAKM |
| | | VKSYLQQHNIPQREVVDTTGLNQSHLSQHLNKGTPMKTQKRAALYTWYVRKQREVAQQ |
| | | FTHAGQGGLIEEPTGDELPTKKGRRNRFKWGPASQQILFQAYERQKNPSKEERETLVE |
| | | ECNRAECIQRGVSPSQAQGLGSNLVTEVRVYNWFANRRKEEAFRHKLAMTCRDEFPTM |
| | | VFPSGQISQASALAPAPPQVLPQAPAPAPAPAMVSALAQAPAPVPVLAPGPPQAVAPP |
| | | APKPTQAGEGTLSEALLQLQFDDEDLGALLGNSTDPAVFTDLASVDNSEFQQLLNQGI |
| | | PVAPHTTEPMLMEYPEAITRLVTGAQRPPDPAPAPLGAPGLPNGLLSGDEDESSIADM |
| | | DFSALLSQISS |
| 300 | IRAG1_CD2 | TTTPAPRPPTPAPTIASQPLSLRPEACWQVIWMMAAVMLVLTVVLGLYKRKKQRSRRM |
| | PrimeR | VSKLSQLQTELLAALLESGLSKEALLQALGEPGPYLLAGEGPLDKGESCGGGRGELAE |
| | | LPNGLGETRGSEDETDDDGEDFTPPILKELENLSPEEAAHQKAVVETLLQEDPWRVAK |
| | | MVKSYLQQHNIPQREVVDTTGLNQSHLSQHLNKGTPMKTQKRAALYTWYVRKQREVAQ |
| | | QFTHAGQGGLIEEPTGDELPTKKGRRNRFKWGPASQQILFQAYERQKNPSKEERETLV |
| | | EECNRAECIQRGVSPSQAQGLGSNLVTEVRVYNWFANRRKEEAFRHKLAMTCRDEFPT |
| | | MVFPSGQISQASALAPAPPQVLPQAPAPAPAPAMVSALAQAPAPVPVLAPGPPQAVAP |
| | | PAPKPTQAGEGTLSEALLQLQFDDEDLGALLGNSTDPAVFTDLASVDNSEFQQLLNQG |
| | | IPVAPHTTEPMLMEYPEAITRLVTGAQRPPDPAPAPLGAPGLPNGLLSGDEDFSSIAD |
| | | MDFSALLSQISS |
| 301 | Notch1_ext_Notch1 | TTTPAPRPPTPAPTIASQPLSLRPEACEMYVAAAAFVLLFFVGCGVLLSRKRRRQHGQ |
| | PrimeR | LWFPEGFKVSEASKKKRREPLGEDSVGLKPLKNAMVSKLSQLQTELLAALLESGLSKE |
| | | ALLQALGEPGPYLLAGEGPLDKGESCGGGRGELAELPNGLGETRGSEDETDDDGEDFT |
| | | PPILKELENLSPEEAAHQKAVVETLLQEDPWRVAKMVKSYLQQHNIPQREVVDTTGLN |
| | | QSHLSQHLNKGTPMKTQKRAALYTWYVRKQREVAQQFTHAGQGGLIEEPTGDELPTKK |
| | | GRRNRFKWGPASQQILFQAYERQKNPSKEERETLVEECNRAECIQRGVSPSQAQGLGS |
| | | NLVTEVRVYNWFANRRKEEAFRHKLAMTCRDEFPTMVFPSGQISQASALAPAPPQVLP |
| | | QAPAPAPAPAMVSALAQAPAPVPVLAPGPPQAVAPPAPKPTQAGEGTLSEALLQLQFD |
| | | DEDLGALLGNSTDPAVFTDLASVDNSEFQQLLNQGIPVAPHTTEPMLMEYPEAITRLV |
| | | TGAQRPPDPAPAPLGAPGLPNGLLSGDEDESSIADMDFSALLSQISS |
| 302 | Notch1_stop PrimeR | TTTPAPRPPTPAPTIASQPLSLRPEACEMYVAAAAFVLLFFVGCGVLLSRKRRRQHGQ |
| | | LWFPEGFKVSEASKKKRREPLGEDSVGLKPLKNAMVSKLSQLQTELLAALLESGLSKE |
| | | ALLQALGEPGPYLLAGEGPLDKGESCGGGRGELAELPNGLGETRGSEDETDDDGEDFT |
| | | PPILKELENLSPEEAAHQKAVVETLLQEDPWRVAKMVKSYLQQHNIPQREVVDTTGLN |
| | | QSHLSQHLNKGTPMKTQKRAALYTWYVRKQREVAQQFTHAGQGGLIEEPTGDELPTKK |
| | | GRRNRFKWGPASQQILFQAYERQKNPSKEERETLVEECNRAECIQRGVSPSQAQGLGS |
| | | NLVTEVRVYNWFANRRKEEAFRHKLAMTCRDEFPTMVFPSGQISQASALAPAPPQVLP |
| | | QAPAPAPAPAMVSALAQAPAPVPVLAPGPPQAVAPPAPKPTQAGEGTLSEALLQLQFD |
| | | DEDLGALLGNSTDPAVFTDLASVDNSEFQQLLNQGIPVAPHTTEPMLMEYPEAITRLV |
| | | TGAQRPPDPAPAPLGAPGLPNGLLSGDEDFSSIADMDFSALLSQISS |

1. A priming receptor comprising, in an N-terminus to C-terminus direction:
a. optionally, an extracellular antigen-binding domain having a binding affinity for an antigen;
b. an STS, SNORC, SEM4F, MCP, PLAT2, MMEL1, PLXC1, CSTN2, SIGL9, IRAG1, TIGIT, TNR21, ZP3, SUSD4, DCBD1, HMR1, DISP1, IRPL1, ECSCR, or PLXC1 transmembrane domain (TMD) comprising one or more ligand-inducible proteolytic cleavage sites; and
c. an intracellular domain comprising a human or humanized transcriptional effector, wherein binding of the antigen to the extracellular antigen-binding domain results in cleavage at the one or more ligand-inducible proteolytic cleavage sites.

2. The priming receptor of claim 1, further comprising a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JMD).

3. A priming receptor comprising, in an N-terminus to C-terminus direction:
a. optionally, an extracellular antigen-binding domain having a binding affinity for an antigen;
b. a transmembrane domain comprising one or more ligand-inducible proteolytic cleavage sites;
c. a PLXA2, NPTN, ALK, DSCAM, CD2, EPHA1, ERMAP, ERBB2, CEA20, KIRR1, C163A, PTPRF, CSPG4, SLAF1, or TMIG3 juxtamembrane domain (JMD); and
d. an intracellular domain comprising a human or humanized transcriptional effector, wherein binding of the antigen to the extracellular antigen-binding domain results in cleavage at the one or more ligand-inducible proteolytic cleavage sites.

4. The priming receptor of claim 3, further comprising an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, IRAG1, HMR1, DISP1, IRPL1, ECSCR, PLXC1, or NOTCH1 transmembrane domain comprising one or more antigen-inducible proteolytic cleavage sites.

5. A priming receptor comprising, in an N-terminus to C-terminus direction:
a. optionally, an extracellular antigen-binding domain having a binding affinity for an antigen;
b. an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1, or NOTCH1 transmembrane domain comprising one or more ligand-inducible proteolytic cleavage sites;
c. a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JMD); and
d. an intracellular domain comprising a human or humanized transcriptional effector, wherein binding of the antigen to the extracellular antigen-binding domain results in cleavage at the one or more ligand-inducible proteolytic cleavage sites.

6. The priming receptor of any one of claims 1-5, wherein the transmembrane domain comprises a sequence set forth in at least one of SEQ ID NOs: 1-21.

7. The priming receptor of any one of claims 1-6, wherein the juxtamembrane domain comprises a sequence set forth in at least one of SEQ ID NOs: 22-39.

8. The priming receptor of any one of claims 1-7, wherein the transmembrane domain and juxtamembrane domain comprise a combination of at least one of: NOTCH1-PLXA2, CLSTN1-CD2, NOTCH1-NPTN, NOTCH1-ALK, SNORC-NOTCH1, SEM4F-NOTCH2, STS-NOTCH2, MCP-NOTCH1, DCBD1-NOTCH2, IRAG1-C163A, HMR1-NOTCH1, MCP-NOTCH1, DISP1-NOTCH2, IRPL1-NOTCH1, ECSCR-NOTCH1, NOTCH1-PTPRF, NOTCH1-CSPG4, SEM4F-NOTCH2, IRAG1-CD2, SIGL9-CD2, IRAG1-NOTCH1, NOTCH1-SLAF1, NOTCH1-TMIG3, NOTCH1-C163A, and PLXC1-EPHA1.

9. The priming receptor of claim 8, wherein the transmembrane domain and juxtamembrane domain comprise a sequence set forth in at least one of SEQ ID NOs: 40-56.

10. The priming receptor of any one of claims 1-9, wherein the extracellular antigen-binding domain is a monoclonal antibody, a neutral antibody, an antagonistic antibody, an agonist antibody, a polyclonal antibody, an afucosylated antibody, a human antibody, a humanized antibody, a chimeric antibody, a full-length antibody, and an scFv.

11. The priming receptor of claim 10, wherein the extracellular antigen-binding domain is an scFv.

12. The priming receptor of any one of claims 1-11, wherein the transcriptional effector domain comprises an HNF1a/p65 domain or a Gal4/VP64 domain.

13. The priming receptor of claim 12, wherein the transcriptional effector domain comprises the sequence as set forth in SEQ ID NO: 80, 81, or 82.

14. The priming receptor of any one of claims 1-13, wherein the priming receptor further comprises a first hinge domain positioned between the first extracellular antigen-binding domain and the first transmembrane domain.

15. The priming receptor of claim 14, wherein the hinge domain comprises a CD8α hinge domain or a truncated CD8α hinge domain.

16. The priming receptor of any one of claims 1-15, wherein the priming receptor comprises a sequence as set forth in at least one of SEQ ID NO: 281-300.

17. The priming receptor of any one of claims 1-16, wherein the priming receptor induces expression of a chimeric antigen receptor (CAR) after binding of the extracellular antigen-binding domain to the antigen.

18. A system comprising a first chimeric polypeptide and a second chimeric polypeptide, wherein
a. the first chimeric polypeptide comprises the priming receptor of any one of claims 1-17; and
b. the second chimeric polypeptide comprises a chimeric antigen receptor (CAR) comprising a second extracellular antigen-binding domain.

19. The system of claim 18, wherein the CAR comprises, from N-terminus to C-terminus:
a. the second extracellular antigen-binding domain;
b. a second transmembrane domain;
c. optionally an intracellular co-stimulatory domain; and
d. an intracellular activation domain.

20. The system of any one of claims 18 or 19, wherein the CAR comprises a second hinge domain.

21. The system of claim 20, wherein the second hinge domain comprises a CD8α hinge domain or a truncated CD8α hinge domain.

22. The system of any one of claims 18-21, wherein the second transmembrane domain comprises a CD8α transmembrane domain.

23. The system of any one of claims 18-22, wherein the intracellular co-stimulatory domain comprises a 4-1BB domain.

24. The system of any one of claims 18-23, wherein the intracellular activation domain comprises a CD3ζ domain.

25. The system of any one of claims 18-24, wherein the system further comprises a self-excising 2A peptide (P2A).

26. The system of any one of claims 18-25, wherein the P2A is at the C-terminus of the chimeric antigen receptor.

27. The system of any one of claims 18-36, wherein the P2A is at the C-terminus of the priming receptor.

28. The system of any one of claims 18-36, wherein binding of the priming receptor to a first antigen on a target cell induces activation of the priming receptor and expression of the chimeric antigen receptor and wherein binding of the chimeric antigen receptor to a second antigen on a target cell modulates the activity of the immune cell.

29. The system of claim 28, wherein the target cell is a human cell.

30. The system of claim 28 or 29, wherein the target cell is a cancer cell.

31. The system of claim 30, wherein the cancer is a solid cancer or a liquid cancer.

32. The system of claim 30 or 31, wherein the cancer is ovarian cancer, fallopian cancer, primary peritoneal cancer, uterine cancer, mesothelioma, cervical cancer, or pancreatic cancer.

33. A recombinant nucleic acid system comprising at least one nucleotide sequence encoding the priming receptor according to any one of claims 1-17.

34. A recombinant nucleic acid system comprising at least one nucleotide sequence encoding the system according to any one of claims 18-32.

35. The recombinant nucleic acid of claim 34, wherein the recombinant nucleic acid system comprises an inducible promoter operably linked to the nucleotide sequence encoding the chimeric antigen receptor.

36. The recombinant nucleic acid of claim 34 or 35, wherein the recombinant nucleic acid system further comprises a constitutive promoter operably linked to the nucleotide sequence encoding the priming receptor.

37. The recombinant nucleic acid of any one of claims 34-36, wherein the recombinant nucleic acid system further comprises an inducible promoter operably linked to the nucleotide sequence encoding the chimeric antigen receptor and a constitutive promoter operably linked to the nucleotide sequence encoding the priming receptor.

38. The recombinant nucleic acid of any one of claim 34-37, wherein the nucleic acid system comprises, in a 5′ to 3′ direction,
a. the constitutive promoter;
b. the nucleotide sequence encoding the priming receptor;
c. the inducible promoter; and
d. the nucleotide sequence encoding the chimeric antigen receptor.

39. The recombinant nucleic acid of any one of claim 34-37, wherein the nucleic acid comprises, in a 5′ to 3′ direction,
a. the inducible promoter;
b. the nucleotide sequence encoding the chimeric antigen receptor;
c. the constitutive promoter; and
d. the nucleotide sequence encoding the priming receptor.

40. The recombinant nucleic acid of any one of claim 34-39, wherein the nucleic acid system further comprises a 5′ homology directed repair arm and a 3′ homology directed repair arm complementary to an insertion site in a host cell chromosome.

41. The recombinant nucleic acid of any one of claims 34-40, wherein the recombinant nucleic acid system further comprises a self-excising 2A peptide (P2A).

42. The recombinant nucleic acid of any one of claims 34-41, wherein the P2A is at the 3′ end of the chimeric antigen receptor.

43. The recombinant nucleic acid of any one of claims 34-42, wherein the P2A is at the 3′ end of the priming receptor.

44. The recombinant nucleic acid of any one of claims 34-43, wherein the recombinant nucleic acid system further comprises a woodchuck hepatitis virus post-translational regulatory element (WPRE).

45. The recombinant nucleic acid of claim 44, wherein the WPRE is at the 3′ end of the nucleotide sequence encoding the chimeric antigen receptor and at the 5′ end of the nucleotide sequence encoding the priming receptor or wherein the WPRE is at the 3′ end of the nucleotide sequence encoding the priming receptor and at the 5′ end of the nucleotide sequence encoding the chimeric antigen receptor.

46. The recombinant nucleic acid of any one of claims 34-45, wherein the nucleic acid system is incorporated into an expression cassette and/or an expression vector.

47. The recombinant nucleic acid of claim 46, wherein the expression vector is a non-viral vector.

48. A non-viral vector comprising the recombinant nucleic acid system of any one of claims 34-47.

49. The non-viral vector of claim 48, wherein the 5′ and 3′ ends of the recombinant nucleic acid system comprise nucleotide sequences that are homologous to genomic sequences flanking an insertion site in a genome of a cell, optionally a primary cell.

50. The non-viral vector of claim 49, wherein the insertion site is at a T Cell Receptor Alpha Constant (TRAC) locus or a genomic safe harbor (GSH) locus.

51. An immune cell comprising:
a. the priming receptor of any one of claims 1-17;
b. the system of any one of claims 18-32;
c. a recombinant nucleic acid system of any one of claims 33-47; and/or
d. the non-viral vector of any one of claims 48-50.

52. The cell of claim 51, wherein the immune cell is a primary human immune cell.

53. The cell of claim 51 or 52, wherein the primary immune cell is an autologous immune cell.

54. The cell of any one of claims 52-53, wherein the primary immune cell is a natural killer (NK) cell, a T cell, a CD8+ T cell, a CD4+ T cell, a primary T cell, or a T cell progenitor.

55. The cell of any one of claims 52-54, wherein the primary immune cell is a primary T cell.

56. The cell of any one of claims 52-55, wherein the primary immune cell is a primary human T cell.

57. The cell of any one of claims 52-56, wherein the primary immune cell is virus-free or does not comprise a viral vector for introducing the recombinant nucleic acid system.

58. The cell of any one of claims 52-57, further comprising obtaining the immune cell from a patient and introducing the system, the recombinant nucleic acid system and/or the non-viral vector in vitro.

59. A primary immune cell comprising at least one recombinant nucleic acid comprising a priming receptor comprising an extracellular antigen-binding domain, an STS, SNORC, SEM4F, MCP, PLAT2, MMEL1, PLXC1, CSTN2, SIGL9, IRAG1, TIGIT, TNR21, ZP3, SUSD4, DCBD1, HMR1, DISP1, IRPL1, ECSCR, or PLXC1 transmembrane domain comprising one or more antigen-inducible proteolytic cleavage sites, a juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

60. A primary immune cell comprising at least one recombinant nucleic acid comprising a priming receptor comprising an extracellular antigen-binding domain, a transmembrane domain comprising one or more antigen-inducible proteolytic cleavage sites, a PLXA2, NPTN, ALK, DSCAM, CD2, EPHA1, ERMAP, ERBB2, CEA20, KIRR1, C163A, PTPRF, CSPG4, SLAF1, or TMIG3 juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

61. A primary immune cell comprising at least one recombinant nucleic acid comprising a priming receptor comprising an extracellular antigen-binding domain, an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1 or NOTCH1 transmembrane domain comprising one or more antigen-inducible proteolytic cleavage sites, a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JDM), and a transcription factor; and a chimeric antigen receptor inserted into a target region of the genome of the primary immune cell, and wherein the primary immune cell does not comprise a viral vector for introducing the recombinant nucleic acid into the primary immune cell.

62. A viable, virus-free, primary cell comprising a ribonucleoprotein complex (RNP)-recombinant nucleic acid complex, wherein the RNP comprises a nuclease domain and a guide RNA, wherein recombinant nucleic acid comprises a priming receptor comprises an STS, SNORC, SEM4F, MCP, PLAT2, MMEL1, PLXC1, CLSTN1, CSTN2, SIGL9, IRAG1, TIGIT, TNR21, ZP3, SUSD4, DCBD1, HMR1, DISP1, IRPL1, ECSCR, PLXC1 or NOTCH1 transmembrane domain comprising one or more antigen-inducible proteolytic cleavage sites; a PLXA2, NPTN, ALK, DSCAM, CD2, EPHA1, ERMAP, ERBB2, CEA20, KIRR1, C163A, PTPRF, CSPG4, SLAF1, TMIG3, NOTCH1, or NOTCH2 juxtamembrane domain (JDM), and a transcription factor; and a chimeric antigen receptor, and wherein the 5′ and 3′ ends of the recombinant nucleic acid comprise nucleotide sequences that are homologous to genomic sequences flanking an insertion site in the genome of the primary cell.

63. A population of cells comprising a plurality of the immune cell of any one of claims 51-62.

64. A pharmaceutical composition comprising the immune cell of any one of claims 51-62 or the population of cells of claim 63, and a pharmaceutically acceptable excipient.

65. A pharmaceutical composition comprising the recombinant nucleic acid of any one of claims 33-47 or the non-viral vector of any one of claims 48-50, and a pharmaceutically acceptable excipient.

66. A method of editing an immune cell, comprising:
a. providing a ribonucleoprotein complex (RNP)-recombinant nucleic acid complex, wherein the RNP comprises a nuclease domain and a guide RNA, wherein the recombinant nucleic acid comprises the recombinant nucleic acid of any one of claims 33-47, and wherein the 5′ and 3′ ends of the recombinant nucleic acid comprise nucleotide sequences that are homologous to genomic sequences flanking an insertion site in the genome of the immune cell;
b. non-virally introducing the RNP-recombinant nucleic acid complex into the immune cell, wherein the guide RNA specifically hybridizes to a target region of the genome of the primary immune cell, and wherein the nuclease domain cleaves the target region to create the insertion site in the genome of the immune cell; and
c. editing the immune cell via insertion of the recombinant nucleic acid of any one of claims 33-47 into the insertion site in the genome of the immune cell.

67. The method of claim 66, wherein non-virally introducing comprises electroporation.

68. The method of claim 66 or 67, wherein the nuclease domain comprises a CRISPR-associated endonuclease (Cas), optionally a Cas9 nuclease.

69. The method of any one of claims 66-68, wherein the target region of the genome of the cell is a T Cell Receptor Alpha Constant (TRAC) locus or a genomic safe harbor (GSH).

70. The method of any one of claims 66-69, wherein the recombinant nucleic acid is a double-stranded recombinant nucleic acid or a single-stranded recombinant nucleic acid.

71. The method of any one of claims 66-70, wherein the recombinant nucleic acid is a linear recombinant nucleic acid or a circular recombinant nucleic acid, optionally wherein the circular recombinant nucleic acid is a plasmid.

72. The method of any one of claims 66-71, wherein the immune cell is a primary human immune cell.

73. The method of any one of claims 66-72, wherein the primary immune cell is an autologous immune cell.

74. The method of any one of claims 66-73, wherein the primary immune cell is a natural killer (NK) cell, a T cell, a CD8+ T cell, a CD4+ T cell, a primary T cell, or a T cell progenitor.

75. The method of any one of claims 66-74, wherein the primary immune cell is a primary T cell.

76. The method of any one of claims 66-75, wherein the primary immune cell is a primary human T cell.

77. The method of any one of claims 66-76, wherein the immune cell is virus-free.

78. The method of any one of claims 66-77, further comprising obtaining the immune cell from a patient and introducing the recombinant nucleic acid in vitro.

79. A method of treating a disease in a subject comprising administering the immune cell of any one of claims 51-63 or the pharmaceutical composition of claims 64 or 65 to the subject.

80. The method of claim 79, wherein the disease is cancer.

81. The method of claim 80, wherein the cancer is a solid cancer or a liquid cancer.

82. The method of claim 79 or 80, wherein the cancer is ovarian cancer, fallopian cancer, primary peritoneal cancer, uterine cancer, mesothelioma, cervical cancer, or pancreatic cancer.

83. The method of any one of claims 79-82, further comprising administering an immunotherapy to the subject concurrently with the immune cell or subsequently to the immune cell.

84. A method of killing, disabling, depleting, or inhibiting a target cell in a subject comprising administering the immune cell of any one of claims 51-63 to the subject, wherein the immune cell kills, disables, depletes, or inhibits the target cell.

85. The method of claim 83 or 84, wherein the target cell is a cancer cell.

86. A method of inducing expression of a chimeric antigen receptor with a priming receptor in an immune cell comprising:
a. obtaining an immune cell comprising:
i. the system of any one of claims 18-32;
ii. the recombinant nucleic acid of any one of claims 33-47; and/or
iii. the non-viral vector of any one of claims 48-50; and
b. contacting the immune cell with a target cell expressing a first antigen to the priming receptor, wherein binding of the priming receptor to the first antigen on the target cell induces activation of the priming receptor and expression of the chimeric antigen receptor.

87. A method of modulating the activity of an immune cell comprising:
a. obtaining an immune cell comprising:
i. the system of any one of claims 18-32;
ii. a recombinant nucleic acid system of any one of claims 33-47; and/or
iii. the non-viral vector of any one of claims 48-50; and
b. contacting the immune cell with a target cell expressing a first antigen and a second antigen, wherein binding of the priming receptor to the first antigen on the target cell induces activation of the priming receptor and expression of the chimeric antigen receptor and wherein binding of the chimeric antigen receptor to the second antigen on the target cell modulates the activity of the immune cell.
